(19) United States
(12) Patent Application Publication
(10) Pub. No.: US 2025/0032585 A1
(43) Pub. Date: Jan. 30, 2025

(54) Effective Doses of CNP Conjugates

(71) Applicant: Ascendis Pharma Growth Disorders A/S, Hellerup (DK)

(72) Inventors: Kennett Sprogøe, Hellerup (DK); Mads Jens Kjelgaard-Hansen, Hellerup (DK)

(21) Appl. No.: 18/708,151

(22) PCT Filed: Dec. 12, 2022
(86) PCT No.: PCT/EP2022/085414

§ 371 Date: May 7, 2024

(30) Foreign Application Priority Data
Dec. 13, 2021 (EP) 21214054.5
Nov. 14, 2022 (EP) 22207210.0

(51) Int. Cl. A61K 38/22; A61K 9/00; A61P 19/08

(52) U.S. Cl. CPC A61K 38/2242; A61K 9/0019; A61P 19/08

(57) ABSTRACT

The present invention relates to unit dosage forms comprising a CNP conjugate or pharmaceutically acceptable salt thereof.

The present invention relates to unit dosage forms comprising a CNP conjugate or pharmaceutically acceptable salt thereof.

Achondroplasia (ACH) is a genetic disorder which occurs due to an autosomal dominant mutation in the fibroblast-growth-factor-receptor 3 (FGFR3) gene, that causes an abnormality of cartilage formation and results in dwarfism. C-type Natriuretic Peptide (CNP) is a hormone that binds and activates the peptide receptor B (NPR-B) resulting in the inhibition of FGFR3 downstream signaling. This in turn triggers endochondral growth and skeletal overgrowth, as observed in both mice and humans overexpressing CNP. Overproduction of CNP in the cartilage or continuous delivery of CNP through intravenous (iv) infusion normalizes the dwarfism of achondroplasic mice, suggesting that administration of CNP at supraphysiological levels is a strategy for treating ACH. The human prepro-CNP which comprises 126 amino acids is further cleaved by furin to yield CNP-53. The CNP-53 possesses biological activity but is typically processed by an unknown mechanism to the biologically active 22 amino acids form in circulation, i.e. CNP-22. The bioactivity of CNP is tightly regulated and its clearance from the plasma is very rapid. Administration of CNP to patients and animals with ACH has been shown to stimulate endochondral bone growth and improve adverse skeletal manifestations of the disease (Lorget et al., Am. J. Hum. Genet. 91, 1108-1114, 2012; Wendt et al., J. Pharmacol. Exp. Ther. 353, 132-149, 2015) and recently vosoritide (VOXZOGO™) was approved for treatment of achondroplasia.

The biology of CNP has been known for many years, but the use of CNP as a pharmaceutical product has been limited by major shortcomings of the native CNP. Native CNP has a half-life of only 2 to 3 minutes in humans, and renal clearance and degradation by neutral endopeptidase (NEP) makes it difficult to maintain an efficacious therapeutic drug exposure. Vosoritide is a daily CNP analog, with increased NEP resistance, and an associated half-life of about 21.0 (±4.7) to 27.9 (±9.9) minutes after subcutaneous administration (VOXZOGO™ Prescribing Information, Reference ID: 4891169, November 2021). Vosoritide is rapidly absorbed after administration and reaches a peak concentration (Cₘax) of about 4.71 (±2.32) to 7.18 (±9.65) ng/mL after approximately 15 minutes after administration of a 15 μg/kg subcutaneous dose (VOXZOGO™ Prescribing Information, Reference ID: 4891169, November 2021). Cyclic guanosine monophosphate (cGMP) is a marker of NPR-B receptor engagement, the pharmacological receptor for CNP. cGMP can be measured both in blood and urine and it is a biomarker of systemic CNP pharmacological activity. An increase in urinary cGMP concentrations from pre-dose baseline were observed within the first four hours post-dose, with a maximum level at 2 hours post-dose, after vasoritide administration to pediatric patients with achondroplasia. Exposure-response analyses showed that vosoritide activity measured by urinary cGMP was near saturation at the 15 μg/kg daily dose. (VOXZOGO™ Prescribing Information, Reference ID: 4891169, November 2021). Furthermore, transient decreases in blood pressure were observed in the clinical studies of vosoritide. As a result, subjects with significant cardiac or vascular disease and patients on anti-hypertensive medicinal products were excluded from participation in vosoritide clinical trials. Due to the risk of low blood pressure, caregivers and patients were informed that vosoritide may lower blood pressure after administration. Also, prior to vosoritide administration, the patient should have adequate food intake and within the hour prior to administration, the patient should drink approximately 8 to ounces (240 to 300 mL) of fluid (VOXZOGO™ Prescribing Information, Reference ID: 4891169, November 2021).

In the clinical trials, decreased blood pressure was observed in 8 (13%) of the 60 subjects treated with a total of 11 events of transient decrease in blood pressure compared to 3 (5%) of 61 subjects on placebo. Two out of 60 (3%) vosoritide-treated subjects each had one symptomatic episode of decreased blood pressure with vomiting and/or dizziness compared to 0 of 61 (0%) subjects on placebo. Daily administration of vasoritide also led to the increase from baseline in serum collagen type X marker (CXM), an endochondral ossification biomarker and remains elevated beyond 24 months. This is consistent with the fact that treatment with vosoritide for 52 weeks resulted in a treatment difference in the change from baseline in annual growth velocity (AGV) of 1.57 cm/year after 52 weeks of treatment when compared to placebo (VOXZOGO™ Prescribing Information, Reference ID: 4891169, November 2021). The hypotensive effects of CNP agonists are mediated through the NPR-B receptor and relate to the Cₘₐₓ of the peptide. cGMP is a good biomarker of CNP target engagement and thus it also serves as a biomarker of hypotensive risk. In the Phase 2 study of vosoritide, a transient dose-dependent increase in urinary cGMP concentration, a biomarker of systemic vosoritide pharmacologic activity, from the pre-dose level to at least 2 hours after the dose was administered was observed. A dose-dependent increase in the median serum CXM concentration from the pre-treatment level was also observed and remained elevated through day 183. In all cohorts, the median serum CXM concentrations were similar and remained elevated from month 12 through month 24. This finding indicates that exposure to vosoritide at the 15 μg/kg dose results in a sustained and maximum response in serum CXM concentration and it is consistent with the fact that the 15 gg/kg dose provides the near-maximum treatment effect with regard to annualized growth velocity for this daily CNP analogue (Savarirayan et al., N. Engl. J. Med. 381, 25-35, 2019). Higher doses thus are unlikely to result in additional growth, but may be associated with higher systemic vosoritide pharmacological activity and thus carry higher hypotensive risk.

It is therefore important to identify a dose of a sustained-release CNP conjugate that improves the pharmacological window and enables effective growth promoting effects with a substantial lower hypotensive potential based on the absence of hypotensive episodes.

It is thus an object of the present invention to at least partially overcome the shortcomings described above.

This object is achieved with a unit dosage form comprising a therapeutically effective amount of a CNP conjugate or pharmaceutically acceptable salt thereof in which a CNP moiety is reversibly conjugated to a polymeric moiety.

Within the meaning of the present invention the terms are used as follows.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “antimicrobial” refers to a chemical substance, such as a chemical substance that kills or inhibits the growth of microorganisms, such as bacteria, fungi, yeasts, protozoans, molds and/or destroys viruses.

As used herein, the term “buffer” or “buffering agent” refers to a chemical compound that maintains the pH in a desired range. Physiologically tolerated buffers are, for example, sodium phosphate, succinate, histidine, bicarbonate, citrate, acetate, sulfate, nitrate, chloride and pyruvate. Antacids such as Mg(OH)₂ or ZnCO₃ may be also used.

As used herein, the term “CNP” refers to all CNP polypeptides, in certain embodiments from mammalian species, such as from human and mammalian species, in particular from human and murine species, as well as their variants, analogs, orthologs, homologs and derivatives and fragments thereof, that are characterized by regulating the growth, proliferation and differentiation of cartilaginous growth plate chondrocytes. The term “CNP” also includes all CNP variants, analogs, orthologs, homologs, derivatives and fragments thereof. The CNP variants, analogs, orthologs, homologs, derivatives and fragments thereof as disclosed in WO 2009/067639 A2 and WO 2010/135541 A2 are herewith incorporated by reference.

As used herein, the term “CNP polypeptide variant” refers to a polypeptide from the same species that differs from a reference CNP polypeptide. Generally, differences are limited so that the amino acid sequence of the reference and the variant are closely similar overall and, in many regions, identical. In certain embodiments, CNP polypeptide variants are at least 70%, 80%, 90%, or 95% identical to a reference CNP polypeptide. By a polypeptide having an amino acid sequence at least, for example, 95% “identical” to a query amino acid sequence, it is intended that the amino acid sequence of the subject polypeptide is identical to the query sequence except that the subject polypeptide sequence may include up to five amino acid alterations per each 100 amino acids of the query amino acid sequence. These alterations of the reference sequence may occur at the amino (N-terminal) or carboxy terminal (C-terminal) positions of the reference amino acid sequence or anywhere between those terminal positions, interspersed either individually among residues in the reference sequence or in one or more contiguous groups within the reference sequence. The query sequence may be an entire amino acid sequence of the reference sequence or any fragment specified as described herein. Such CNP polypeptide variants may be naturally occurring variants, such as naturally occurring allelic variants encoded by one of several alternate forms of a CNP occupying a given locus on a chromosome or an organism, or isoforms encoded by naturally occurring splice variants originating from a single primary transcript. Alternatively, a CNP polypeptide variant may be a variant that is not known to occur naturally and that can be made by mutagenesis techniques known in the art. It is known in the art that one or more amino acids may be deleted from the N-terminus or C-terminus of a bioactive peptide or protein without substantial loss of biological function. Such N- and/or C-terminal deletions are also encompassed by the term CNP polypeptide variant.

It is also recognized by one of ordinary skill in the art that some amino acid sequences of CNP polypeptides can be varied without significant effect on the structure or function of the peptide. Such mutants include deletions, insertions, inversions, repeats, and substitutions selected according to general rules known in the art so as to have little effect on activity. For example, guidance concerning how to make phenotypically silent amino acid substitutions is provided in Bowie et al. (1990), Science 247:1306-1310, which is hereby incorporated by reference in its entirety, wherein the authors indicate that there are two main approaches for studying the tolerance of the amino acid sequence to change.

As used herein, the term “CNP analog” refers to CNP of different and unrelated organisms which perform the same functions in each organism, but which did not originate from an ancestral structure that the organisms' ancestors had in common. Instead, analogous CNPs arose separately and then later evolved to perform the same or similar functions. In other words, analogous CNP polypeptides are polypeptides with quite different amino acid sequences that perform the same biological activity, namely regulating the growth, proliferation and differentiation of cartilaginous growth plate chondrocytes.

As used herein, the term “CNP ortholog” refers to CNP within two different species which sequences are related to each other via a common homologous CNP in an ancestral species, but which have evolved to become different from each other.

As used herein, the term “CNP homolog” refers to CNP of different organisms which perform the same functions in each organism, and which originate from an ancestral structure that the organisms' ancestors had in common. In other words, homologous CNP polypeptides are polypeptides with quite similar amino acid sequences that perform the same biological activity, namely regulating the growth, proliferation and differentiation of cartilaginous growth plate chondrocytes. In certain embodiments, CNP polypeptide homologs may be defined as polypeptides exhibiting at least 40%, 50%, 60%, 70%, 80%, 90% or 95% identity to a reference CNP polypeptide.

Thus, a CNP polypeptide may be, for example: (i) one in which at least one of the amino acid residues is substituted with a conserved or non-conserved amino acid residue, in certain embodiments, a conserved amino acid residue, and such substituted amino acid residue may or may not be one encoded by the genetic code; and/or (ii) one in which at least one of the amino acid residues includes a substituent group; and/or (iii) one in which the CNP polypeptide is fused with another compound, such as a compound to increase the half-life of the polypeptide (for example, polyethylene glycol); and/or (iv) one in which additional amino acids are fused to the CNP polypeptide, such as an IgG Fc fusion region peptide or leader or secretory sequence or a sequence which is employed for purification of the above form of the polypeptide or a pre-protein sequence.

As used herein, the term “CNP polypeptide fragment” refers to any peptide comprising a continuous span of a part of the amino acid sequence of a CNP polypeptide.

More specifically, a CNP polypeptide fragment comprises at least 6, such as at least 8, at least or at least 17 consecutive amino acids of a CNP polypeptide. A CNP polypeptide fragment may additionally be described as sub-genuses of CNP polypeptides comprising at least 6 amino acids, wherein “at least 6” is defined as any integer between 6 and the integer representing the C-terminal amino acid of a CNP polypeptide. Further included are species of CNP polypeptide fragments at least 6 amino acids in length, as described above, that are further specified in terms of their N-terminal and C-terminal positions. Also encompassed by the term “CNP polypeptide fragment” as individual species are all CNP polypeptide fragments, at least 6 amino acids in length, as described above, that may be particularly specified by a N-terminal and C-terminal position. That is, every combination of a N-terminal and C-terminal position that a fragment at least 6 contiguous amino acid residues in length could occupy, on any given amino acid sequence of a CNP polypeptide.

As the term CNP includes the above-described variants, analogs, orthologs, homologs, derivatives and fragments of CNP, all references to specific positions within a reference sequence also include the equivalent positions in the variants, analogs, orthologs, homologs, derivatives and fragments of a CNP moiety, even if not explicitly mentioned.

Naturally occurring CNP-22 (SEQ ID NO:1) has the following sequence:

GLSKGCFGLKLDRIGSMSGLGC,

wherein the cysteines at position 6 and 22 are connected through a disulfide-bridge.

In certain embodiments, the term “CNP” also refers to the following peptide sequences:

SEQ ID NO: 2 (CNP-53):
DLRVDTKSRAAWARLLQEHPNARKYKGANKKGLSKGCFGLKLDRIGSMSGLGC;
SEQ ID NO: 3 (G-CNP-53):
GDLRVDTKSRAAWARLLQEHPNARKYKGANKKGLSKGCFGLKLDRIGSMSGLGC;
SEQ ID NO: 4 (M-CNP-53):
MDLRVDTKSRAAWARLLQEHPNARKYKGANKKGLSKGCFGLKLDRIGSMSGLGC;
SEQ ID NO: 5 (P-CNP-53):
PDLRVDTKSRAAWARLLQEHPNARKYKGANKKGLSKGCFGLKLDRIGSMSGLGC;
SEQ ID NO: 6 (CNP-53 M48N):
DLRVDTKSRAAWARLLQEHPNARKYKGANKKGLSKGCFGLKLDRIGSNSGLGC;
SEQ ID NO: 7 (CNP-53 Δ15-31):
DLRVDTKSRAAWARGLSKGCFGLKLDRIGSMSGLGC;
SEQ ID NO: 8 (CNP-52):
LRVDTKSRAAWARLLQEHPNARKYKGANKKGLSKGCFGLKLDRIGSMSGLGC;
SEQ ID NO: 9 (CNP-51):
RVDTKSRAAWARLLQEHPNARKYKGANKKGLSKGCFGLKLDRIGSMSGLGC;
SEQ ID NO: 10 (CNP-50):
VDTKSRAAWARLLQEHPNARKYKGANKKGLSKGCFGLKLDRIGSMSGLGC;
SEQ ID NO: 11 (CNP-49):
DTKSRAAWARLLQEHPNARKYKGANKKGLSKGCFGLKLDRIGSMSGLGC;
SEQ ID NO: 12 (CNP-48):
TKSRAAWARLLQEHPNARKYKGANKKGLSKGCFGLKLDRIGSMSGLGC;
SEQ ID NO: 13 (CNP-47):
KSRAAWARLLQEHPNARKYKGANKKGLSKGCFGLKLDRIGSMSGLGC;
SEQ ID NO: 14 (CNP-46):
SRAAWARLLQEHPNARKYKGANKKGLSKGCFGLKLDRIGSMSGLGC;
SEQ ID NO: 15 (CNP-45):
RAAWARLLQEHPNARKYKGANKKGLSKGCFGLKLDRIGSMSGLGC;
SEQ ID NO: 16 (CNP-44):
AAWARLLQEHPNARKYKGANKKGLSKGCFGLKLDRIGSMSGLGC;
SEQ ID NO: 17 (CNP-44 Δ14-22):
AAWARLLQEHPNAGLSKGCFGLKLDRIGSMSGLGC;
SEQ ID NO: 18 (CNP-44 Δ15-22):
AAWARLLQEHPNARGLSKGCFGLKLDRIGSMSGLGC;
SEQ ID NO: 19 (CNP-43):
AWARLLQEHPNARKYKGANKKGLSKGCFGLKLDRIGSMSGLGC;
SEQ ID NO: 20 (CNP-42):
WARLLQEHPNARKYKGANKKGLSKGCFGLKLDRIGSMSGLGC;
SEQ ID NO: 21 (CNP-41):
ARLLQEHPNARKYKGANKKGLSKGCFGLKLDRIGSMSGLGC;
SEQ ID NO: 22 (CNP-40):
RLLQEHPNARKYKGANKKGLSKGCFGLKLDRIGSMSGLGC;
SEQ ID NO: 23 (CNP-39):
LLQEHPNARKYKGANKKGLSKGCFGLKLDRIGSMSGLGC;
SEQ ID NO: 24 (CNP-38):
LQEHPNARKYKGANKKGLSKGCFGLKLDRIGSMSGLGC,
wherein the cysteines at position 22 and 38 are connected through a disulfide-bridge;
SEQ ID NO: 25 (CNP-37):
QEHPNARKYKGANKKGLSKGCFGLKLDRIGSMSGLGC;
SEQ ID NO: 26 (CNP-37 Q1pQ, wherein pQ = pyroglutamate):
pQEHPNARKYKGANKKGLSKGCFGLKLDRIGSMSGLGC;
SEQ ID NO: 27 (G-CNP-37):
GQEHPNARKYKGANKKGLSKGCFGLKLDRIGSMSGLGC;
SEQ ID NO: 28 (P-CNP-37):
PQEHPNARKYKGANKKGLSKGCFGLKLDRIGSMSGLGC;
SEQ ID NO: 29 (M-CNP-37):
MQEHPNARKYKGANKKGLSKGCFGLKLDRIGSMSGLGC;
SEQ ID NO: 30 (PG-CNP-37):
PGQEHPNARKYKGANKKGLSKGCFGLKLDRIGSMSGLGC;
SEQ ID NO: 31 (MG-CNP-37):
MGQEHPNARKYKGANKKGLSKGCFGLKLDRIGSMSGLGC;
SEQ ID NO: 32 (CNP-37 M32N):
QEHPNARKYKGANKKGLSKGCFGLKLDRIGSNSGLGC;
SEQ ID NO: 33 (G-CNP-37 M32N):
GQEHPNARKYKGANKKGLSKGCFGLKLDRIGSNSGLGC;
SEQ ID NO: 34 (G-CNP-37 K14Q):
GQEHPNARKYKGANQKGLSKGCFGLKLDRIGSMSGLGC;
SEQ ID NO: 35 (G-CNP-37 K14P):
GQEHPNARKYKGANPKGLSKGCFGLKLDRIGSMSGLGC;
SEQ ID NO: 36 (G-CNP-37 K14Q, Δ15):
GQEHPNARKYKGANQGLSKGCFGLKLDRIGSMSGLGC;
SEQ ID NO: 37 (G-CNP-37 K14Q, K15Q):
GQEHPNARKYKGANQQGLSKGCFGLKLDRIGSMSGLGC;
SEQ ID NO: 38 (CNP-36):
EHPNARKYKGANKKGLSKGCFGLKLDRIGSMSGLGC;
SEQ ID NO: 39 (CNP-35):
HPNARKYKGANKKGLSKGCFGLKLDRIGSMSGLGC;
SEQ ID NO: 40 (CNP-34):
PNARKYKGANKKGLSKGCFGLKLDRIGSMSGLGC;
SEQ ID NO: 41 (CNP-33):
NARKYKGANKKGLSKGCFGLKLDRIGSMSGLGC;
SEQ ID NO: 42 (CNP-32):
ARKYKGANKKGLSKGCFGLKLDRIGSMSGLGC;
SEQ ID NO: 43 (CNP-31):
RKYKGANKKGLSKGCFGLKLDRIGSMSGLGC;
SEQ ID NO: 44 (CNP-30):
KYKGANKKGLSKGCFGLKLDRIGSMSGLGC;
SEQ ID NO: 45 (CNP-29):
YKGANKKGLSKGCFGLKLDRIGSMSGLGC;
SEQ ID NO: 46 (CNP-28):
KGANKKGLSKGCFGLKLDRIGSMSGLGC;
SEQ ID NO: 47 (GHKSEVAHRF-CNP-28):
GHKSEVAHRFKGANKKGLSKGCFGLKLDRIGSMSGLGC;
SEQ ID NO: 48 (CNP-27):
GANKKGLSKGCFGLKLDRIGSMSGLGC;
SEQ ID NO: 49 (CNP-27 K4Q, K5Q):
GANQQGLSKGCFGLKLDRIGSMSGLGC;
SEQ ID NO: 50 (CNP-27 K4R, K5R):
GANRRGLSKGCFGLKLDRIGSMSGLGC;
SEQ ID NO: 51 (CNP-27 K4P, K5R):
GANPRGLSKGCFGLKLDRIGSMSGLGC;
SEQ ID NO: 52 (CNP-27 K4S, K5S):
GANSSGLSKGCFGLKLDRIGSMSGLGC;
SEQ ID NO: 53 (CNP-27 K4P, K5R):
GANGANPRGLSRGCFGLKLDRIGSMSGLGC;
SEQ ID NO: 54 (CNP-27 K4R, K5R, K9R):
GANRRGLSRGCFGLKLDRIGSMSGLGC;
SEQ ID NO: 55 (CNP-27 K4R, K5R, K9R, M22N):
GANRRGLSRGCFGLKLDRIGSNSGLGC;
SEQ ID NO: 56 (P-CNP-27 K4R, K5R, K9R):
PGANRRGLSRGCFGLKLDRIGSMSGLGC;
SEQ ID NO: 57 (M-CNP-27 K4R, K5R, K9R):
MGANRRGLSRGCFGLKLDRIGSMSGLGC;
SEQ ID NO: 58 (HSA fragment-CNP-27):
GHKSEVAHRFKGANKKGLSKGCFGLKLDRIGSMSGLG;
SEQ ID NO: 59 (HSA fragment-CNP-27 M22N):
GHKSEVAHRFKGANKKGLSKGCFGLKLDRIGSNSGLGC;
SEQ ID NO: 60 (M-HSA fragment-CNP-27):
MGHKSEVAHRFKGANKKGLSKGCFGLKLDRIGSMSGLGC;
SEQ ID NO: 61 (P-HSA fragment-CNP-27):
PGHKSEVAHRFKGANKKGLSKGCFGLKLDRIGSMSGLGC;
SEQ ID NO: 62 (CNP-26):
ANKKGLSKGCFGLKLDRIGSMSGLGC;
SEQ ID NO: 63 (CNP-25):
NKKGLSKGCFGLKLDRIGSMSGLGC;
SEQ ID NO: 64 (CNP-24):
KKGLSKGCFGLKLDRIGSMSGLGC;
SEQ ID NO: 65 (CNP-23):
KGLSKGCFGLKLDRIGSMSGLGC;
SEQ ID NO: 66 (R-CNP-22):
RGLSKGCFGLKLDRIGSMSGLGC;
SEQ ID NO: 67 (ER-CNP-22):
ERGLSKGCFGLKLDRIGSMSGLGC;
SEQ ID NO: 68 (R-CNP-22 K4R):
RGLSRGCFGLKLDRIGSMSGLGC;
SEQ ID NO: 69 (ER-CNP-22 4KR):
ERGLSRGCFGLKLDRIGSMSGLGC;
SEQ ID NO: 70 (RR-CNP-22):
RRGLSRGCFGLKLDRIGSMSGLGC;
SEQ ID NO: 71 (HRGP fragment-CNP-22):
GHHSHEQHPHGANQQGLSKGCFGLKLDRIGSMSGLGC;
SEQ ID NO: 72 (HRGP fragment-CNP-22):
GAHHPHEHDTHGANQQGLSKGCFGLKLDRIGSMSGLGC;
SEQ ID NO: 73 (HRGP fragment-CNP-22):
GHHSHEQHPHGANPRGLSKGCFGLKLDRIGSMSGLGC;
SEQ ID NO: 74 (IgG1(Fc) fragment-CNP-22):
GQPREPQVYTLPPSGLSKGCFGLKLDRIGSMSGLGC;
SEQ ID NO: 75 (HSA fragment-CNP-22):
GQHKDDNPNLPRGANPRGLSKGCFGLKLDRIGSMSGLGC;
SEQ ID NO: 76 (HSA fragment-CNP-22):
GERAFKAWAVARLSQGLSKGCFGLKLDRIGSMSGLGC;
SEQ ID NO: 77 (osteocrin NPR C inhibitor fragment-CNP22):
FGIPMDRIGRNPRGLSKGCFGLKLDRIGSMSGLGC;
SEQ ID NO: 78 (FGF2 heparin-binding domain fragment-CNP22):
GKRTGQYKLGSKTGPGPKGLSKGCFGLKLDRIGSMSGLGC;
SEQ ID NO: 79 (IgG1(Fc) fragment-CNP-22 K4R):
GQPREPQVYTGANQQGLSRGCFGLKLDRIGSMSGLGC;
SEQ ID NO: 80 (HSA fragment-CNP-22 K4R):
GVPQVSTSTGANQQGLSRGCFGLKLDRIGSMSGLGC;
SEQ ID NO: 81 (fibronectin fragment-CNP-22 K4R):
GQPSSSSQSTGANQQGLSRGCFGLKLDRIGSMSGLGC;
SEQ ID NO: 82 (fibronectin fragment-CNP-22 K4R):
GQTHSSGTQSGANQQGLSRGCFGLKLDRIGSMSGLGC;
SEQ ID NO: 83 (fibronectin fragment-CNP-22 K4R):
GSTGQWHSESGANQQGLSRGCFGLKLDRIGSMSGLGC;
SEQ ID NO: 84 (zinc finger fragment-CNP-22 K4R):
GSSSSSSSSSGANQQGLSRGCFGLKLDRIGSMSGLGC;
SEQ ID NO: 85 (CNP-21):
LSKGCFGLKLDRIGSMSGLGC;
SEQ ID NO: 86 (CNP-20):
SKGCFGLKLDRIGSMSGLGC;
SEQ ID NO: 87 (CNP-19):
KGCFGLKLDRIGSMSGLGC;
SEQ ID NO: 88 (CNP-18):
GCFGLKLDRIGSMSGLGC;
SEQ ID NO: 89 (CNP-17):
CFGLKLDRIGSMSGLGC;
SEQ ID NO: 90 (BNP fragment-CNP-17-BNP fragment):
SPKMVQGSGCFGLKLDRIGSMSGLGCKVLRRH;
SEQ ID NO: 91 (CNP-38 L1G):
GQEHPNARKYKGANKKGLSKGCFGLKLDRIGSMSGLGC;
SEQ ID NO: 92 (Ac-CNP-37; wherein Ac = acetyl):
Ac-QEHPNARKYKGANKKGLSKGCFGLKLDRIGSMSGLGC;
SEQ ID NO: 93:
QEHPNARX₁YX₂GANX₃X₄GLSX₅GCFGLX₆LDRIGSMSGLGC,
wherein X₁, X₂, X₃, X₄, X₅ and X₆ are independently of each other selected from the group
consisting of K, R, P, S and Q, with the provision that at least one of X₁, X₂, X₃, X₄, X₅
and X₆ is selected from the group consisting of R, P, S and Q; in certain embodiments,
X₁, X₂, X₃, X₄, X₅ and X₆ are selected from the group consisting of K and R, with the
provision that at least one of X₁, X₂, X₃, X₄, X₅ and X₆ is R;
SEQ ID NO: 94:
QEHPNARKYKGANX₁X2GLSX₃GCFGLX₄LDRIGSMSGLGC,
wherein X₁, X₂, X₃ and X₄ are independently of each other selected from the group
consisting of K, R, P, S and Q, with the provision that at least one of X₁, X₂, X₃ and X₄
is selected from the group consisting of R, P, S and Q; in certain embodiments, X₁, X₂,
X₃ and X₄ are selected from K and R, with the provision that at least one of X₁, X₂, X₃
and X₄ is R;
SEQ ID NO: 95:
QEHPNARKYKGANX₁X₂GLSKGCFGLKLDRIGSMSGLGC,
wherein X₁X₂ are selected from the group consisting of KR, RK, KP, PK, SS, RS, SR, QK, QR,
KQ, RQ, RR and QQ.

It is understood that also the equivalents of the cysteines in positions 22 and 38 of SEQ ID NO:24 are connected through a disulfide-bridge in SEQ ID NOs: 2 to 95.

The term “CNP” also includes poly(amino acid) conjugates which have a sequence as described above, but have a backbone that comprises both amide and non-amide linkages, such as ester linkages, like for example depsipeptides. Depsipeptides are chains of amino acid residues in which the backbone comprises both amide (peptide) and ester bonds. Accordingly, the term “side chain” as used herein refers either to the moiety attached to the alpha-carbon of an amino acid moiety, if the amino acid moiety is connected through amine bonds such as in polypeptides, or to any carbon atom-comprising moiety attached to the backbone of a poly(amino acid) conjugate, such as for example in the case of depsipeptides. In certain embodiments, the term “CNP” refers to polypeptides having a backbone formed through amide (peptide) bonds.

As used herein, the term “ring moiety” refers to the stretch of consecutive amino acid residues of the CNP drug or moiety that is located between two cysteine residues that form an intramolecular disulfide bridge or between homologous amino acid residues which are connected through a chemical linker. Preferably, the ring moiety is located between two cysteine residues that form an intramolecular disulfide bridge. These two cysteines correspond to the cysteines at position 22 and position 38 in the sequence of CNP-38 (SEQ ID NO:24).

Accordingly, amino acids 23 to 37 are located in said ring moiety, if the CNP drug or moiety has the sequence of CNP-38.

Independently of the length of the CNP moiety, the sequence of the ring moiety of wild-type CNP is FGLKLDRIGSMSGLG (SEQ ID NO:96).

As the term CNP includes the above-described variants, analogs, orthologs, homologs, derivatives and fragments of CNP, the term “ring moiety” also includes the corresponding variants, analogs, orthologs, homologs, derivatives and fragments of the sequence of SEQ ID NO:96. Accordingly, all references to specific positions within a reference sequence also include the equivalent positions in variants, analogs, orthologs, homologs, derivatives and fragments of a CNP moiety, even if not explicitly mentioned.

As used herein, the term “C₁—₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀, C₁₋₂₀ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the term “C₂₋₁₀ alkenyl”, “C₂₋₂₀ alkenyl” or “C₂₋₅₀ alkenyl” alone or in combination means a straight-chain or branched hydrocarbon moiety comprising at least one carbon-carbon double bond having 2 to 10, 2 to 20 or 2 to 50 carbon atoms. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are in certain embodiments, selected from the group consisting of

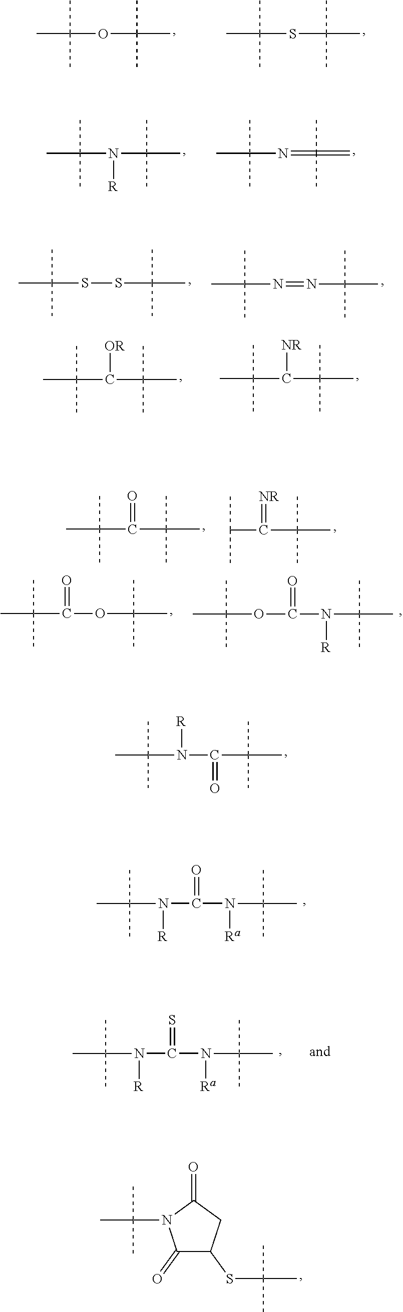

wherein
dashed lines indicate attachment to the remainder of the moiety or reagent; and —R and —R^{a} are independently of each other selected from the group consisting of —H, methyl, ethyl, propyl, butyl, pentyl and hexyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbomane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings, in certain embodiments of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure:

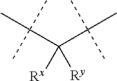

means that R^{x} and R^{y} form the following structure:

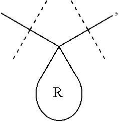

wherein R is C₃₋₁₀ cycloalkyl or 3- to 10-membered heterocyclyl.

It is also understood that the phrase “the pair R^{x}/R^{y} is joined together with the atoms to which they are attached to form a ring A” in relation with a moiety of the structure:

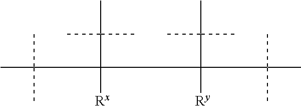

means that R^{x} and R^{y} form the following structure:

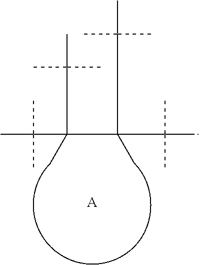

As used herein, the term “dose” or “unit dose” refers to the predetermined amount of the drug, such as CNP, administered at one time to produce a certain degree of biological response in a patient. The dose of a drug is governed by its inherent potency and in this case, it is a therapeutic dose or therapeutic unit dose.

As used herein, the term “dosage form” refers to the physical form that comprises the active pharmaceutical ingredient in combination with selected additional ingredients or excipients and which is intended to be delivered to sites of action within the body by various routes of drug administration. It also refers to the physical form in which a precise mixture of active pharmaceutical ingredients and excipients are presented to help administration and delivery to the sites of action, achieve rapid onset of action and improve bioavailability. As used herein, the term “unit dosage form” refers to a dosage form configured for a single administration to a patient. For example, a unit dosage form can be a single vial or the container containing an amount of drug suitable for a single administration.

As used herein, the term “dosage regimen” is the combination of dose, and frequency with which a drug is administered. Dosage regimen can also include a route of administration (e.g., subcutaneous) and/or duration of administration (e.g., until a patient reaches 18 years old or ephiphyseal closure). Administration of a dosage regimen may maintain a steady-state serum concentration of CNP, in which peaks, troughs and area under the curve over a defined interval remain with defined margins of fluctuation and/or the ratio of peaks to troughs does not exceed a defined threshold.

As used herein, the term “drug” refers to a substance used in the treatment, cure, prevention, or diagnosis of a disease or used to otherwise enhance physical or mental well-being. If a drug, such as CNP, is conjugated to another moiety, the moiety of the resulting product that originated from the drug is referred to as “drug moiety”.

As used herein, the term “excipient” refers to compounds administered together with the drug or drug conjugate, for example, buffering agents, isotonicity modifiers, preservatives, stabilizers, anti-adsorption agents, oxidation protection agents, or other auxiliary agents. However, in some cases, one excipient may have dual or triple functions. The term “excipient” may also refer to a diluent, adjuvant, or vehicle with which the drug or drug conjugate, is administered. Such pharmaceutical excipient can be sterile liquids, such as water and oils, including those of petroleum, animal, vegetable or synthetic origin, including, but not limited to peanut oil, soybean oil, mineral oil, sesame oil and the like. Water is a preferred excipient when the pharmaceutical formulation is administered orally. Saline and aqueous dextrose are preferred excipients when the pharmaceutical formulation is administered intravenously or subcutaneously. Saline solutions and aqueous dextrose and glycerol solutions are in certain embodiments, employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, water, ethanol and the like. The pharmaceutical formulation can also contain minor amounts of wetting or emulsifying agents, pH buffering agents, like, for example, acetate, succinate, Tris (tris(hydroxymethyl)aminomethane), carbonate, phosphate, HEPES (4-(2-hydroxyethyl)-1-piperazineethanesulfonic acid), MES (2-(N-morpholino)ethanesulfonic acid), or can contain detergents, like Tween®, poloxamers, poloxamines, CHAPS, Igepal®, or amino acids like, for example, glycine, lysine, or histidine. These pharmaceutical formulations can take the form of solutions, suspensions, emulsions, tablets, pills, capsules, powders, sustained-release formulations and the like. The pharmaceutical formulation can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such formulations will contain a therapeutically effective amount of the drug or drug moiety, together with a suitable amount of excipient so as to provide the form for proper administration to the patient. The formulation should suit the mode of administration.

As used herein, the term “formulation” or “pharmaceutical formulation” refers to a formulation containing one or more CNP conjugates and one or more excipients, as well as any product which results, directly or indirectly, from combination, complexation or aggregation of any two or more of the ingredients of the composition, or from dissociation of one or more of the ingredients, or from other types of reactions or interactions of one or more of the ingredients. Accordingly, the pharmaceutical formulations of the present invention encompass any formulation or composition made by admixing one or more CNP conjugates and a pharmaceutically acceptable excipient such as a buffering agent and bulking agent.

As used herein, the term “free form” of a drug refers to the drug in its unmodified, pharmacologically fully active form, e.g. after being released from the CNP conjugate or pharmaceutically acceptable salt thereof.

As used herein, the term “functional group” means a group of atoms which can react with other groups of atoms. Functional groups include, but are not limited, to the following groups: carboxylic acid (—(C═O)OH), primary or secondary amine (—NH₂, —NH—), maleimide, thiol (—SH), sulfonic acid (—(O═S═O)OH), carbonate, carbamate (—O(C═O)N<), hydroxyl (—OH), aldehyde (—(C═O)H), ketone (—(C═O)—), hydrazine (>N—N<), isocyanate, isothiocyanate, phosphoric acid (—O(P═O)OHOH), phosphonic acid (—O(P═O)OHH), haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. It is generally preferred that halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

As used herein, the term “isotonicity agent” refers to a compound that minimizes pain, irritation and tissue damage that can result from cell damage due to osmotic pressure differences between the injected solution and plasma.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety, such as a CNP moiety, is released from a conjugate as a drug, such as CNP.

It is understood that if the sequence or chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R′)—” can be attached to two moieties or interrupting a moiety either as “—C(O)N(R¹)—” or as “—N(R¹)C(O)—”. Similarly, a moiety:

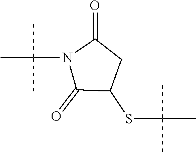

can be attached to two moieties or can interrupt a moiety either as:

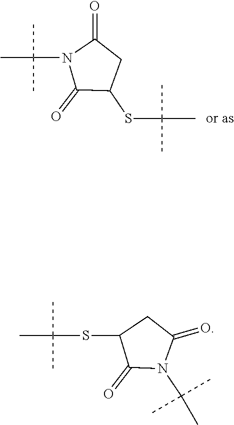

In case the CNP moiety comprises one or more acidic or basic groups, the unit dosage form comprises also their corresponding pharmaceutically or toxicologically acceptable salts, in particular their pharmaceutically utilizable salts. Thus, the CNP moieties comprising one or more acidic groups can be present and used, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, and other salts or amines known to the person skilled in the art. CNP moieties comprising one or more basic groups, i.e. groups which can be protonated, can be present and can be used in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to the person skilled in the art. For the person skilled in the art further methods are known for converting the basic group into a cation like the alkylation of an amine group resulting in a positively charged ammonium group and an appropriate counterion of the salt. If the CNP moieties simultaneously comprise acidic and basic groups, the pharmaceutical formulations according to the present invention also include, in addition to the salt forms mentioned, inner salts or betaines (zwitterions). The respective salts can be obtained by customary methods which are known to the person skilled in the art like, for example by contacting these conjugates with an organic or inorganic acid or base in a solvent or dispersant, or by anion exchange or cation exchange with other salts. The unit dosage form according to the present invention also includes all salts of the CNP conjugates which, owing to low physiological compatibility, are not directly suitable for use in pharmaceuticals but which can be used, for example, as intermediates for chemical reactions or for the preparation of pharmaceutically acceptable salts.

As used herein, the term “patient” refers to a subject amenable to treatment or prophylaxis according to the invention, particularly a human subject.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “physiological conditions” refers to an aqueous buffer at pH 7.4 and 37° C.

The term “polypeptide” as used herein refers to a chain of at least 2 and up to and including 50 amino acid monomer moieties linked by peptide (amide) linkages. Only for CNP drugs and CNP moieties also the sequences having more than 50 amino acids will be referred to as “polypeptide” for simplification.

As used herein, the term “preservative” refers to a chemical substance that has antimicrobial effects and prevents chemical degradation.

As used herein, the term “protein” refers to a chain of more than 50 amino acid monomer moieties linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. It is understood that a polymer may also comprise one or more other chemical groups and/or moieties, such as, for example, one or more functional groups. In certain embodiments, a soluble polymer has a molecular weight of at least 0.5 kDa, e.g. a molecular weight of at least 1 kDa, a molecular weight of at least 2 kDa, a molecular weight of at least 3 kDa or a molecular weight of at least 5 kDa. If the polymer is soluble, in certain embodiments it has a molecular weight of at most 1000 kDa, such as at most 750 kDa, such as at most 500 kDa, such as at most 300 kDa, such as at most 200 kDa, such as at most 100 kDa.

It is understood that also a protein or a polypeptide is a polymer in which the amino acids are the repeating structural units, even though the side chains of each amino acid may be different.

As used herein, the term “polymeric” or “polymeric moiety” means a reagent or a moiety comprising one or more polymers or polymer moieties. A polymeric reagent or moiety may optionally also comprise one or more other moiety/moieties, which are in certain embodiments selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

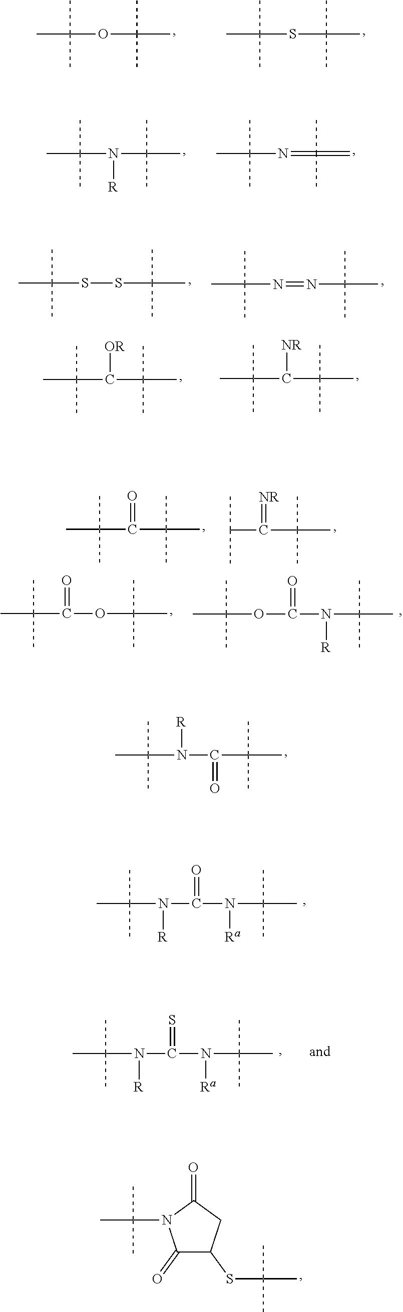

wherein
dashed lines indicate attachment to the remainder of the moiety or reagent, and —R and —R^{a} are independently of each other selected from the group consisting of —H, methyl, ethyl, propyl, butyl, pentyl and hexyl.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “PEG-based” in relation to a moiety or reagent means that said moiety or reagent comprises PEG. In certain embodiments, a PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent are other moieties selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

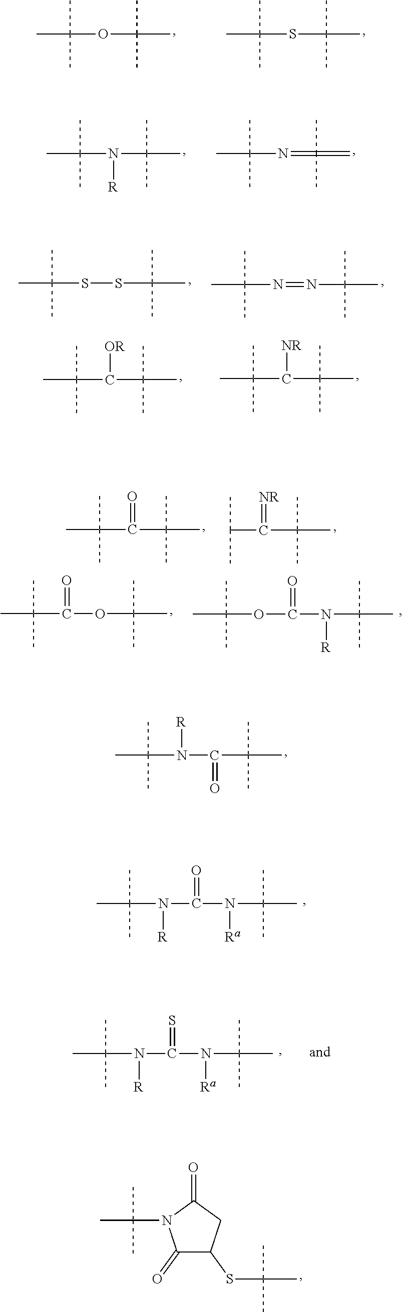

wherein
dashed lines indicate attachment to the remainder of the moiety or reagent, and —R and —R^{a} are independently of each other selected from the group consisting of —H, methyl, ethyl, propyl, butyl, pentyl and hexyl.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent and in certain embodiments, all ethylene glycol units of said moiety or reagent are present in one block; the remaining weight percentage of the PEG-based moiety or reagent are other moieties in certain embodiments selected from the following moieties and linkages:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

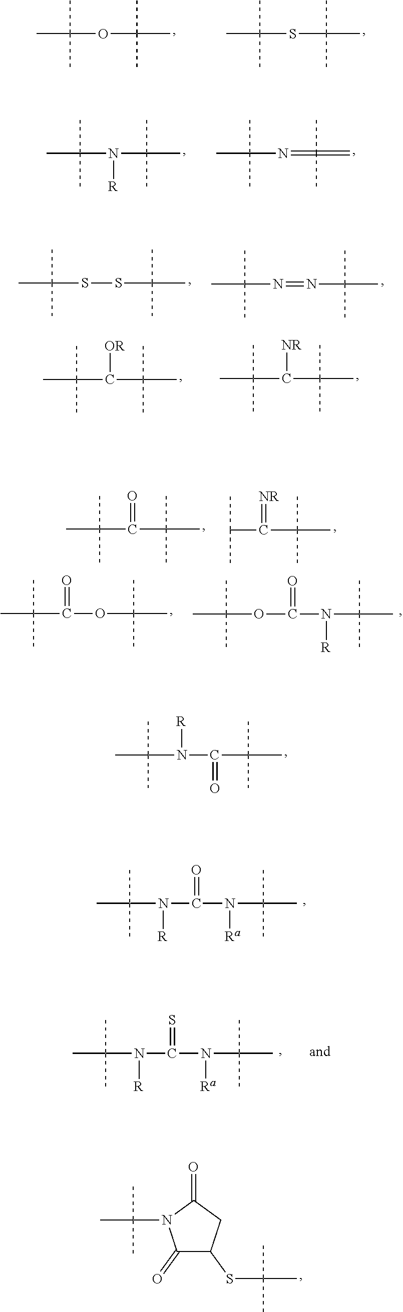

wherein
dashed lines indicate attachment to the remainder of the moiety or reagent, and —R and —R^{a} are independently of each other selected from the group consisting of —H, methyl, ethyl, propyl, butyl, pentyl and hexyl.

As used herein, the term “hyaluronic acid-based comprising at least X % hyaluronic acid” is used accordingly.

It is also recognized by one of ordinary skill in the art that the conjugates of the present invention are prodrugs. As used herein, the term “prodrug” refers to a drug moiety, such as a CNP moiety, reversibly and covalently conjugated to a polymeric moiety, such as —Z, through a reversible linker moiety. A prodrug releases the reversibly and covalently bound drug moiety in the form of its corresponding drug. In other words, a prodrug is a conjugate comprising a drug moiety, such as a CNP moiety, which is covalently and reversibly conjugated to a polymeric moiety via a reversible linker moiety, which covalent and reversible conjugation of the polymeric moiety to the reversible linker moiety is either direct or through a spacer. Such prodrugs or conjugates release the formerly conjugated drug moiety in the form of a free drug.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “reversible linkage” is a linkage that is cleavable, in the absence of enzymes under physiological conditions (aqueous buffer at pH 7.4, 37° C.) with a half-life ranging from one hour to six months, such as from one hour to four months, such as from one hour to three months, from one hour to two months or from one hour to one month. Accordingly, a stable linkage is a linkage having a half-life under physiological conditions (aqueous buffer at pH 7.4, 37° C.) of more than six months.

As used herein, the term “reagent” means a chemical compound which comprises at least one functional group for reaction with the functional group of another chemical compound or drug. It is understood that a drug comprising a functional group (such as a primary or secondary amine or hydroxyl functional group) is also a reagent.

As used herein, the term “reversible linker moiety” is a moiety which is covalently conjugated to a drug moiety, such as a CNP moiety, through a reversible linkage and is also covalently conjugated to a polymeric moiety, such as —Z, wherein the covalent conjugation to said polymeric moiety is either direct or through a spacer moiety, such as -L²-. In certain embodiments, the linkage between —Z and -L²- is a stable linkage. A conjugate comprising a reversible linker moiety can be referred to as a reversible conjugate.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl or C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl.

As used herein, the term “substituted” means that one or more —H atom(s) of a molecule or moiety are replaced by a different atom or a group of atoms, which are referred to as “substituent”.

In certain embodiments, such one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—; —R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4}b), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the one or more substituents are independently of each other selected from the group consisting of halogen, —CN, —COOR^{x1}, —OR^{x1}, —C(O)R^{x1}. —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂—₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4}b), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4}b), —OC(O)N(R^{x4}R^{x4a}), and C₁₋₆ alkyl; wherein C₁₋₆ alkyl is optionally substituted with one or more halogen, which are the same or different;
each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the one or more substituents are independently of each other selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2}, which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician. Within the scope of this invention, therapeutically effective amount relates to dosages that aim to achieve therapeutic effect for an extended period of time, i.e. for at least one day, such as for two days, such as for three days, such as for four days, such as for five days, such as for six days, such as for one week or such as for two weeks.

As used herein, the term “traceless linker” means a reversible linker which upon cleavage releases the drug in its free form.

As used herein, the term “water-soluble” with reference to a polymeric moiety means that when such polymeric moiety is part of the CNP conjugate, at least 1 g of the CNP conjugate comprising such water-soluble polymeric moiety can be dissolved in one liter of water at 20° C. to form a homogeneous solution.

In general, the term “comprise” or “comprising” also encompasses “consist of” or “consisting of”.

The unit dose comprised within the unit dosage form of the present invention depends on the patient's actual body weight.

In certain embodiments, the unit dose ranges from about 12.3 nmol CNP/kg to at least about 37 nmol CNP/kg. In certain embodiments, the unit dose ranges from 12.3 nmol CNP/kg to 36.9 nmol CNP/kg. In certain embodiments, the unit dose ranges is at least 24.6 nmol CNP/kg. In certain embodiments, the unit dose ranges is about 24.6 nmol CNP/kg. In certain embodiments, the unit dose ranges is 24.6 nmol CNP/kg.

In certain embodiments, the unit dose ranges from about 6 pg CNP/kg to at least about 100 pg CNP/kg. In certain embodiments, the unit dose ranges from about 6 pg CNP/kg to about 150 pg CNP/kg.

In certain embodiments, the unit dose comprised within the unit dosage form of the present invention ranges from 6 pg CNP/kg to at least 100 pg CNP/kg. In certain embodiments, the unit dose ranges from 6 pg CNP/kg to 150 pg CNP/kg.

It is understood that “x” pg CNP/kg refers to “x” pg of CNP, i.e. of the CNP moiety comprised within the CNP conjugate, per kilogram of patient's body weight. Similarly, it is understood that “y” nmol CNP/kg refers to “y” nmol of CNP, i.e. of the CNP moiety comprised within the CNP conjugate, per kilogram of patient's body weight.

In certain embodiments, the patient is an adult. In certain embodiments, the patient is a pediatric patient. In certain embodiments, the patient is an infant.

In certain embodiments, the patient's body weight ranges from about 2 kg to about 80 kg. In certain embodiments, the patient's body weight ranges from about 4 kg to about 60 kg. In certain embodiment, the patient's body weight is about 5 kg. In certain embodiment, the patient's body weight is about 9 kg. In certain embodiment, the patient's body weight is about kg. In certain embodiment, the patient's body weight is about 11 kg. In certain embodiment, the patient's body weight is about 12 kg. In certain embodiment, the patient's body weight is about 15 kg. In certain embodiment, the patient's body weight is about 20 kg. In certain embodiment, the patient's body weight is about 30 kg. In certain embodiment, the patient's body weight is about 40 kg. In certain embodiment, the patient's body weight is about 50 kg. In certain embodiment, the patient's body weight is about 60 kg. In certain embodiment, the patient's body weight is about 70 kg. In certain embodiment, the patient's body weight is about 80 kg.

In certain embodiments, the unit dose ranges from 50 μg to 7000 μg of CNP. In certain embodiments, the unit dose ranges from 100 μg to 5000 μg of CNP. In certain embodiments, the unit dose ranges from 100 μg to 3000 μg of CNP. In certain embodiments, the unit dose ranges from 100 μg to 2000 μg of CNP. In certain embodiments, the unit dose ranges from 100 μg to 1000 μg of CNP. In certain embodiments, the unit dose ranges from 150 μg to 750 μg of CNP. In certain embodiments, the unit dose ranges from 150 μg to 500 μg of CNP. In certain embodiments, the unit dose ranges from 150 μg to 350 μg of CNP. In certain embodiments, the unit dose is about 700 pg CNP. In certain embodiments, the unit dose is about 600 pg CNP. In certain embodiments, the unit dose is about 500 pg CNP. In certain embodiments, the unit dose is about 400 pg CNP. In certain embodiments, the unit dose is about 300 pg CNP.

In certain embodiments, the unit dose is about 6 pg CNP/kg. In certain embodiments, the unit dose is about 20 pg CNP/kg. In certain embodiments, the unit dose is about 50 pg CNP/kg. In certain embodiments, the unit dose is about 75 pg CNP/kg. In certain embodiments, the unit dose is about 100 pg CNP/kg. In certain embodiments, the unit dose is about 125 pg CNP/kg. In certain embodiments, the unit dose is about 150 pg CNP/kg.

In certain embodiments, the unit dose is 6 pg CNP/kg. In certain embodiments, the unit dose is pg CNP/kg. In certain embodiments, the unit dose is 50 pg CNP/kg. In certain embodiments, the unit dose is 75 ug CNP/kg. In certain embodiments, the unit dose is 100 ug CNP/kg. In certain embodiments, the unit dose is 125 pg CNP/kg. In certain embodiments, the unit dose is 150 pg CNP/kg.

In certain embodiments, the unit dosage form is liquid. In certain embodiments, the unit dosage form is solid.

Prior to a subcutaneous administration to a patient in need thereof, the solid unit dosage form is reconstituted. Reconstitution of the solid unit dosage form into a reconstituted formulation is done by adding a predefined amount of reconstitution solution to the solid unit dosage form. Therefore, a further aspect of the present invention is a method of reconstituting the solid unit dosage form of the present invention wherein the method comprises the step of
(a) contacting the solid unit dosage form of the present invention with a reconstitution solution.

Reconstitution may take place in the container in which the solid unit dosage form is provided, such as in a vial; syringe such as a dual-chamber syringe; ampoule; cartridge, such as a dual-chamber cartridge; or the solid unit dosage form may be transferred to a different container where is then reconstituted. In certain embodiments, the container in which the reconstitution of the solid unit dosage form takes place is a vial. In certain embodiments, the container in which the reconstitution of the solid unit dosage form takes place is a syringe. In certain embodiments, the container in which the reconstitution of the solid unit dosage form takes place is a dual-chamber syringe. In certain embodiments, the container in which the reconstitution of the solid unit dosage form takes place is a cartridge. In certain embodiments, the container in which the reconstitution of the solid unit dosage form takes place is a dual-chamber cartridge.

In certain embodiments, the solid unit dosage form according to the present invention is provided in a first chamber of the dual-chamber syringe and the reconstitution solution is provided in a second chamber of the dual-chamber syringe.

The reconstitution solution is a sterile liquid, such as water or buffer, which may comprise further additives, such as preservatives and/or antimicrobials.

In certain embodiments, the reconstituted solution comprises one or more preservative and/or antimicrobial and/or antioxidant.

In certain embodiments, the reconstituted solution comprises one or more preservative.

The preservative may be selected from the group consisting of m-cresol, benzoic acid, phenol, methylparaben, ethylparaben, propylparaben, butylparaben, potassium sorbate, chlorobutanol, benzyl alcohol, phenylmercuric nitrate, thimerosal, sorbic acid, potassium sorbate, chlorocresol, benzalkonium chloride, 2-ethoxyethanol, chlorhexidine, chlorobutanol, phenylethyl alcohol, phenylmercuric acetate and mixtures thereof.

In certain embodiments, the preservative is m-cresol. In certain embodiments, the preservative is benzylalcohol. In certain embodiments, the preservative is benzoic acid. In certain embodiments, the preservative is phenol. In certain embodiments, the preservative is methylparaben. In certain embodiments, the preservative is ethylparaben. In certain embodiments, the preservative is propylparaben. In certain embodiments, the preservative is butylparaben. In certain embodiments, the preservative is potassium sorbate. In certain embodiments, the preservative is benzyl alcohol. In certain embodiments, the preservative is phenylmercuric nitrate. In certain embodiments, the preservative is thimerosal. In certain embodiments, the preservative is sorbic acid. In certain embodiments, the preservative is potassium sorbate. In certain embodiments, the preservative is chlorocresol. In certain embodiments, the preservative is benzalkonium chloride. In certain embodiments, the preservative is 2-ethoxyethanol. In certain embodiments, the preservative is chlorhexidine. In certain embodiments, the preservative is chlorbutanol. In certain embodiments, the preservative is phenylethyl alcohol. In certain embodiments, the preservative is phenylmercuric acetate.

In certain embodiments, the preservative has a concentration ranging from 1 to 10 mg/ml. In certain embodiments, the preservative has a concentration ranging from 1.5 to 3.5 mg/ml. In certain embodiments, the preservative has a concentration ranging from 2 to 3 mg/ml.

The antioxidant may be selected from the group consisting of methionine, butylhydroxytoluene, butylhydroxyanisol, tocopherol, propylgallate, ascorbic acid, ethylenediaminetetraacetic acid (EDTA), poly(ethylenimine), vitamin E and mixtures thereof.

In certain embodiments, the preservative is methionine. In certain embodiments, the preservative is butylhydroxytoluene. In certain embodiments, the preservative is butylhydroxyanisol. In certain embodiments, the preservative is tocopherol. In certain embodiments, the preservative is propylgallate. In certain embodiments, the preservative is ethylenediaminetetraacetic acid. In certain embodiments, the preservative is poly(ethylenimine). In certain embodiments, the preservative is vitamin E.

As defined herein, the term “methionine” is intended to encompass both D-methionine and L-methionine, and mixtures thereof. In certain embodiments, the term “methionine” refers to L-methionine. In certain embodiments, the term “methionine” refers to D-methionine. In certain embodiments, the term “methionine” refers to a mixture of D-methionine or L-methionine. In certain embodiments, the term “methionine” refers to L-methionine hydrochloride salt.

As defined herein, the term “EDTA” is intended to encompass all EDTA forms that are known in the art such as EDTA salts, including EDTA metal salts, such as EDTA disodium salt, EDTA dipotassium salt, EDTA calcium salt, EDTA dimagnesium salt or mixtures thereof. In certain embodiments, EDTA refers to EDTA disodium salt. In certain embodiments, the term “EDTA” refers to EDTA dicalcium salt. In certain embodiments, the term “EDTA” refers to EDTA anhydrous.

In certain embodiments, the molar ratio of antioxidant to CNP moiety is from about 0.1:1 to about 100:1. In certain embodiments, the molar ratio of antioxidant to CNP moiety is from about 0.1:1 to about 70:1. In certain embodiments, the molar ratio of antioxidant to CNP moiety is from about 0.1:1 to about 15:1. In certain embodiments, the molar ratio of antioxidant to CNP moiety is from about 1:1 to about 10:1. In certain embodiments, the molar ratio of antioxidant to CNP moiety is from about 3:1 to about 7:1.

In certain embodiments, the reconstituted solution does not comprise an antimicrobial. In certain embodiments, the reconstituted solution comprises one or more excipient.

In certain embodiments, the reconstitution solution is sterile water. In certain embodiments, the reconstitution solution is sterile water comprising 0.7-1.1% benzylalcohol. In certain embodiments, the reconstitution solution is sterile water comprising 0.9% benzylalcohol.

In certain embodiments, the reconstituted solution comprises a pH-modifying agent.

As used herein, the term “pH-modifying agent” refers to a chemical compound that is used to modify the pH of the reconstitution solution.

In certain embodiments, the pH-modifying agent may be an acid or acidic salt thereof. The acid may be selected from the group consisting of acetic acid, citric acid, succinic acid, hydrochloric acid, phosphoric acid, carbonic acid, nitric acid and mixtures thereof.

In certain embodiments, the pH-modifying agent may be a base or basic salt thereof. The base may be selected from the group consisting of Tris (tris(hydroxymethyl)aminomethane), sodium hydroxide, potassium hydroxide, lysine and mixtures thereof.

In certain embodiments, the volume of the reconstitution solution ranges from about 0.1 ml to about 4 ml. In certain embodiments, the volume of the reconstitution solution is about 1 ml, such as about 2 ml, such as about 3 ml or such as about 4 ml.

In certain embodiments, the volume of the reconstitution solution is about 0.79 ml. In certain embodiments, the volume of the reconstitution solution is 0.79 ml. In certain embodiments, the volume of the reconstitution solution is about 1 ml. In certain embodiments, the volume of the reconstitution solution is 1 ml. In certain embodiments, the volume of the reconstitution solution is about 1.1 ml. In certain embodiments, the volume of the reconstitution solution is 1.1 ml. In certain embodiments, the volume of the reconstitution solution is about 1.25 ml. In certain embodiments, the volume of the reconstitution solution is 1.25 ml

It is understood that the volume of the unit dose or injection volume is based on the patient's actual body weight and the concentration of the reconstituted solution. In certain embodiments, the concentration of CNP within the reconstituted solution is not more than 7 mg/ml. In certain embodiments, the concentration of CNP within the reconstituted solution is not less than 0.5 mg/ml. In certain embodiments, the concentration of CNP within the reconstituted solution is 0.75 mg/ml. In certain embodiments, the concentration of CNP within the reconstituted solution is 1 mg/ml. In certain embodiments, the concentration of CNP within the reconstituted solution is 2.2 mg/ml. In certain embodiments, the concentration of CNP within the reconstituted solution is 3.6 mg/ml. In certain embodiments, the concentration of CNP within the reconstituted solution is 4.6 mg/ml. In certain embodiments, the concentration of CNP within the reconstituted solution is 5 mg/ml. In certain embodiments, the concentration of CNP within the reconstituted solution is 5.5 mg/ml.

After reconstitution, a unit dose has a volume of not more than 4 ml. In certain embodiments, the volume of the unit dose ranges from about 0.01 ml to about 1.1 ml. In certain embodiments, the volume of the unit dose ranges from 0.01 ml to 0.75 ml. In certain embodiments, the volume of the unit dose ranges from 0.01 ml to 0.50 ml.

In certain embodiments, the volume of the unit dose is about 0.03 ml. In certain embodiments, the volume of the unit dose is about 0.05 ml. In certain embodiments, the volume of the unit dose is about 0.1 ml. In certain embodiments, the volume of the unit dose is about 0.2 ml. In certain embodiments, the volume of the unit dose is about 0.25 ml. In certain embodiments, the volume of the unit dose is about 0.3 ml. In certain embodiments, the volume of the unit dose is about 0.35 ml. In certain embodiments, the volume of the unit dose is about 0.4 ml. In certain embodiments, the volume of the unit dose is about 0.5 ml. In certain embodiments, the volume of the unit dose is about 0.6 ml. In certain embodiments, the volume of the unit dose is about 0.75 ml. In certain embodiments, the volume of the unit dose is about 1 ml.

In certain embodiments, the patient is an infant and the volume of the unit dose ranges from about 10 μl to 100 μl. In certain embodiments, the patient is an infant and the volume of the unit dose ranges from about 10 μl to 50 μl. In certain embodiments, the patient is an infant and the volume of the unit dose ranges from about 10 μl to 30 gl.

In certain embodiments, the patient is an infant and the volume of the unit dose is about 10 μl. In certain embodiments, the patient is an infant and the volume of the unit dose is about 15 μl. In certain embodiments, the patient is an infant and the volume of the unit dose is about 20 gl. In certain embodiments, the patient is an infant and the volume of the unit dose is 10 μl. In certain embodiments, the patient is an infant and the volume of the unit dose is 15 μl. In certain embodiments, the patient is an infant and the volume of the unit dose is 20 μl.

In certain embodiments, the patient is an infant, the unit dose is 20 pg CNP/kg and the volume of the unit dose is about 10 μl. In certain embodiments, the patient is an infant, the unit dose is pg CNP/kg and the volume of the unit dose is about 15 μl. In certain embodiments, the patient is an infant, the unit dose is 20 μg CNP/kg and the volume of the unit dose is about 20 μl.

In certain embodiments, the unit dose is 6 μg CNP/kg and the volume of the unit dose is 0.06 ml. In certain embodiments, the unit dose is 20 μg CNP/kg and the volume of the unit dose 0.3 ml. In certain embodiments, the unit dose is 50 μg CNP/kg and the volume of the unit dose is 0.2 ml. In certain embodiments, the unit dose is 75 μg CNP/kg and the volume of the unit dose is 0.4 ml. In certain embodiments, the unit dose is 100 μg CNP/kg and the volume of the unit dose is 0.5 ml. In certain embodiments, the unit dose is 150 μg CNP/kg and the volume of the unit dose is 0.5 ml.

In certain embodiments, the pH of the liquid unit dosage form is from about pH 4 to about pH 6. In certain embodiments, the pH of the liquid unit dosage form is from about pH 4.5 to about pH 5.5. In certain embodiments, the pH of the liquid unit dosage form is about 5. In certain embodiments, the pH of the liquid unit dosage form is 5.

In certain embodiments, the unit dosage form of the present invention further comprises a buffering agent, an isotonicity agent and a pH-modifying agent.

In certain embodiments, the buffering agent has a concentration ranging from 1.3 to 57.6 mM in the unit dosage form. In certain embodiments, the buffering agent has a concentration ranging from 1.7 to 33 mM in the unit dosage form. In certain embodiments, the buffering agent has a concentration ranging from 5.1 to 20.3 mM in the unit dosage form. In certain embodiments, the buffering agent has a concentration of about 10 mM in the unit dosage form.

Exemplary buffering agents may be selected from the group consisting of succinic acid, citric acid, lactic acid, acetic acid, glutamic acid, fumaric acid, aspartic acid, glutaric acid, phosphoric acid, histidine, gluconic acid, tartaric acid, malic acid and mixtures thereof. It is clear to the person skilled in the art that the corresponding conjugate bases or salts of the buffering agents such as succinate, citrate, lactate, acetate, glutamate, fumarate, aspartate, glutarate, phosphate, gluconate, tartrate, malate and mixtures thereof, respectively, may also be included.

In certain embodiments, the buffering agent is succinic acid. In certain embodiments, the buffering agent is citric acid. In certain embodiments, the buffering agent is lactic acid. In certain embodiments, the buffering agent is acetic acid. In certain embodiments, the buffering agent is glutamic acid. In certain embodiments, the buffering agent is fumaric acid. In certain embodiments, the buffering agent is aspartic acid. In certain embodiments, the buffering agent is glutaric acid. In certain embodiments, the buffering agent is phosphoric acid. In certain embodiments, the buffering agent is histidine. In certain embodiments, the buffering agent is gluconic acid. In certain embodiments, the buffering agent is tartaric acid. In certain embodiments, the buffering agent is malic acid.

The isotonicity agent may be selected from the group consisting of trehalose, mannitol, sucrose, raffinose, gelatin, lactose, dibasic calcium phosphate, sorbitol, xylitol, glycine, histidine, hydroxyethylstarch, dextrose, dextran, Ficoll®, propylene glycol and mixtures thereof. In certain embodiments, the isotonicity agent may be selected from the group consisting of trehalose, mannitol, sucrose, raffinose, gelatin, lactose, dibasic calcium phosphate, sorbitol, xylitol, glycine, histidine, hydroxyethylstarch, dextrose, dextran, propylene glycol and mixtures thereof.

In certain embodiments, the isotonicity agent is selected from the group consisting of trehalose, sucrose and glycine. In certain embodiments, the isotonicity agent is a non-reducing sugar such as trehalose or sucrose.

In certain embodiments, the isotonicity agent is trehalose.

As defined herein, the term “trehalose” is intended to encompass all salts and hydration states of trehalose, such as trehalose anhydrous or trehalose dihydrate. In certain embodiments, the term “trehalose” refers to trehalose anhydrous. In certain embodiments, the term “trehalose” refers to trehalose dihydrate.

In certain embodiments, the unit dosage form comprises succinic acid and trehalose.

In certain embodiments, the unit dosage form comprises

| | | |
|---|---|---|
| CNP conjugate | 0.9-82.1 | mg/ml |
| succinic acid | 1.3-57.6 | mM |
| trehalose dihydrate | 67-111.6 | mg/ml, |

and has a pH ranging from pH 4.0 to pH 6.0.

In certain embodiments, the unit dosage form comprises:

| | | |
|---|---|---|
| CNP conjugate | 19.8-73.6 | mg/ml |
| succinic acid | 1.7-50 | mM |
| trehalose dihydrate | 63-100 | mg/ml |

and has a pH ranging from pH 4.0 to pH 6.0.

In certain embodiments, the unit dosage form comprises:

| | | |
|---|---|---|
| CNP conjugate | 27.5-50.5 | mg/ml |
| succinic acid | 5.1-20.3 | mM |
| trehalose dihydrate | 67-95 | mg/ml |

and has a pH ranging from pH 4.0 to pH 6.0.

In certain embodiments, the unit dosage form comprises about 8.2 mg/ml CNP conjugate, about 10 mM succinic acid, about 89 mg/ml trehalose dihydrate and optionally Tris and/or hydrochloric acid and has a pH of about 5.

In certain embodiments, the unit dosage form comprises 8.2 mg/ml CNP conjugate, 10 mM succinic acid, 89 mg/ml trehalose dihydrate and optionally Tris and/or hydrochloric acid and has a pH of 5.

In certain embodiments, the unit dosage form comprises about 11 mg/ml CNP conjugate, about 10 mM succinic acid, about 88.5 mg/ml trehalose dihydrate and optionally Tris and/or hydrochloric acid and has a pH of about 5.

In certain embodiments, the unit dosage form comprises 11 mg/ml CNP conjugate, 10 mM succinic acid, 88.5 mg/ml trehalose dihydrate and optionally Tris and/or hydrochloric acid and has a pH of 5.

In certain embodiments, the unit dosage form comprises about 24.2 mg/ml CNP conjugate, about 10 mM succinic acid, about 85 mg/ml trehalose dihydrate and optionally Tris and/or hydrochloric acid and has a pH of about 5.

In certain embodiments, the unit dosage form comprises 24.2 mg/ml CNP conjugate, 10 mM succinic acid, 85 mg/ml trehalose dihydrate and optionally Tris and/or hydrochloric acid and has a pH of 5.

In certain embodiments, the unit dosage form comprises about 39.6 mg/ml CNP conjugate, about 10 mM succinic acid, about 80 mg/ml trehalose dihydrate and optionally Tris and/or hydrochloric acid and has a pH of about 5.

In certain embodiments, the unit dosage form comprises 39.6 mg/ml CNP conjugate, 10 mM succinic acid, 80 mg/ml trehalose dihydrate and optionally Tris and/or hydrochloric acid and has a pH of 5.

In certain embodiments, the unit dosage form comprises about 50.5 mg/ml CNP conjugate, about 10 mM succinic acid, about 77 mg/ml trehalose dihydrate and optionally Tris and/or hydrochloric acid and has a pH of about 5.

In certain embodiments, the unit dosage form comprises 50.5 mg/ml CNP conjugate, 10 mM succinic acid, 77 mg/ml trehalose dihydrate and optionally Tris and/or hydrochloric acid and has a pH of 5.

In certain embodiments, the unit dosage form comprises about 54.9 mg/ml CNP conjugate, about 10 mM succinic acid, about 75 mg/ml trehalose dihydrate and optionally Tris and/or hydrochloric acid and has a pH of about 5.

In certain embodiments, the unit dosage form comprises 54.9 mg/ml CNP conjugate, 10 mM succinic acid, 75 mg/ml trehalose dihydrate and optionally Tris and/or hydrochloric acid and has a pH of about 5.

In certain embodiments, the unit dosage form comprises about 60.4 mg/ml CNP conjugate, about 10 mM succinic acid, about 73 mg/ml trehalose dihydrate and optionally Tris and/or hydrochloric acid and has a pH of about 5.

In certain embodiments, the unit dosage form comprises 60.4 mg/ml CNP conjugate, 10 mM succinic acid, 73 mg/ml trehalose dihydrate and optionally Tris and/or hydrochloric acid and has a pH of 5.

In certain embodiments, the unit dosage form comprises based on the total weight of the solid unit dosage form:

| | | |
|---|---|---|
| CNP conjugate | 8.2-44.4% | (w/w) |
| succinic acid | 0.9-1.2% | (w/w) |
| trehalose dihydrate | 53.7-89.1% | (w/w) |
| Tris | 1.0-1.5% | (w/w). |

In certain embodiments, the unit dosage form comprises, based on the total weight of the solid unit dosage form, about 8.2% (w/w) CNP conjugate, about 1.2% (w/w) succinic acid, about 89.1% (w/w) trehalose dihydrate and about 1.5% (w/w) Tris.

In certain embodiments, the unit dosage form comprises, based on the total weight of the solid unit dosage form, 8.2% (w/w) CNP conjugate, 1.2% (w/w) succinic acid, 89.1% (w/w) trehalose dihydrate and 1.5% (w/w) Tris.

In certain embodiments, the unit dosage form comprises, based on the total weight of the solid unit dosage form, about 10.7% (w/w) CNP conjugate, about 1.2% (w/w) succinic acid, about 86.8% (w/w) trehalose dihydrate and about 1.3% (w/w) Tris.

In certain embodiments, the unit dosage form comprises, based on the total weight of the solid unit dosage form, 10.7% (w/w) CNP conjugate, 1.2% (w/w) succinic acid, 86.8% (w/w) trehalose dihydrate and 1.3% (w/w) Tris.

In certain embodiments, the unit dosage form comprises, based on the total weight of the solid unit dosage form, about 21.6% (w/w) CNP conjugate, about 1.1% (w/w) succinic acid, about 76.1% (w/w) trehalose dihydrate and about 1.2% (w/w) Tris.

In certain embodiments, the unit dosage form comprises, based on the total weight of the solid unit dosage form, 21.6% (w/w) CNP conjugate, 1.1% (w/w) succinic acid, 76.1% (w/w) trehalose dihydrate and 1.2% (w/w) Tris.

In certain embodiments, the unit dosage form comprises, based on the total weight of the solid unit dosage form, about 32.4% (w/w) CNP conjugate, about 1.0% (w/w) succinic acid, about 65.4% (w/w) trehalose dihydrate and about 1.2% (w/w) Tris.

In certain embodiments, the unit dosage form comprises, based on the total weight of the solid unit dosage form, 32.4% (w/w) CNP conjugate, 1.0% (w/w) succinic acid, 65.4% (w/w) trehalose dihydrate and 1.2% (w/w) Tris.

In certain embodiments, the unit dosage form comprises, based on the total weight of the solid unit dosage form, about 38.9% (w/w) CNP conjugate, about 0.9% (w/w) succinic acid, about 59.2% (w/w) trehalose dihydrate and about 1% (w/w) Tris.

In certain embodiments, the unit dosage form comprises, based on the total weight of the solid unit dosage form, 38.9% (w/w) CNP conjugate, 0.9% (w/w) succinic acid, 59.2% (w/w) trehalose dihydrate and 1% (w/w) Tris.

In certain embodiments, the unit dosage form comprises, based on the total weight of the solid unit dosage form, about 41.5% (w/w) CNP conjugate, about 0.9% (w/w) succinic acid, about 56.6% (w/w) trehalose dihydrate and about 1% (w/w) Tris.

In certain embodiments, the unit dosage form comprises, based on the total weight of the solid unit dosage form, 41.5% (w/w) CNP conjugate, 0.9% (w/w) succinic acid, 56.6% (w/w) trehalose dihydrate and 1% (w/w) Tris.

In certain embodiments, the unit dosage form comprises, based on the total weight of the solid unit dosage form, about 44.4% (w/w) CNP conjugate, about 0.9% (w/w) succinic acid, about 53.7% (w/w) trehalose dihydrate and about 1% (w/w) Tris.

In certain embodiments, the unit dosage form comprises, based on the total weight of the solid unit dosage form, 44.4% (w/w) CNP conjugate, 0.9% (w/w) succinic acid, 53.7% (w/w) trehalose dihydrate and 1% (w/w) Tris.

Applicant surprisingly found that upon administration to a patient in need thereof of the unit dosage form of the present invention, the incidence of hypotension is less than 10%, preferably less than 8%, most preferably less than 5%, even more preferably is less than 3%. In certain embodiments, upon administration to a patient in need thereof of the unit dosage form of the present invention, the incidence of hypotension is less than 1%. In certain embodiments, there is no incidence of hypotension.

Also, it was surprisingly found that no treatment emergent anti-CNP antibodies were detected upon treatment with the conjugate of the present invention. In certain embodiments, no anti-CNP binding antibodies have been detected upon 1 to 9 months of repeated weekly exposure to the conjugate of the present invention. In certain embodiments, no anti-CNP binding antibodies have been detected upon 52 weeks of repeated weekly exposure to the conjugate of the present invention.

Moreover, it was surprisingly found that the administration of 100 μg CNP/kg per week to pedriatic patients aged 2 to 10 years, such as 2 to 5 years or such as 5 to 10 years, in need of CNP treatment resulted in similar responses measured as annualized growth velocity.

In certain embodiments, the CNP moiety of the CNP conjugate has the sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25 or SEQ ID NO:30. In certain embodiments, the CNP moiety has the sequence of SEQ ID NO:20, SEQ ID NO:21, SEQ ID NO:22, SEQ ID NO:23, SEQ ID NO:24 or SEQ ID NO:25. In certain embodiments, the CNP moiety has the sequence of SEQ ID NO:20. In certain embodiments, the CNP moiety has the sequence of SEQ ID NO:21. In certain embodiments, the CNP moiety has the sequence of SEQ ID NO:22. In certain embodiments, the CNP moiety has the sequence of SEQ ID NO:23. In certain embodiments, the CNP moiety has the sequence of SEQ ID NO:24. In certain embodiments, the CNP moiety has the sequence of SEQ ID NO:25.

In certain embodiments, the CNP conjugate is of formula (Ia) or (Ib):

Z [symbol] L²-L¹-D)ₓ (Ia),

D [symbol] L¹-L²-Z)_{y} (Ib),
wherein
   -D is a CNP moiety;
   -L¹- is a reversible linker moiety;
   -L²- is a single chemical bond or a spacer moiety;
   —Z is a polymeric moiety;
   x is an integer selected from the group consisting of 1, 2, 3, 4, 5, 6, 7, 8, 9, 10, 11, 12, 13, 14, 15 and 16; and
   y is an integer selected from the group consisting of 1, 2, 3, 4 and 5.
      -D of formula (Ia) or (Ib) is covalently and reversibly conjugated to -L¹-.

In certain embodiments, x of formula (Ia) is an integer selected from the group consisting of 1, 2, 3, 4, 6 and 8. In certain embodiments, x of formula (Ia) is an integer selected from the group consisting of 1, 2, 4 and 6. In certain embodiments, x of formula (Ia) is an integer selected from the group consisting of 1, 4 and 6 and in certain embodiments. x of formula (Ia) is 1.

In certain embodiments, y of formula (Ib) is an integer selected from the group consisting of 2, 3, 4 and 5. In certain embodiments, y of formula (Ib) is an integer selected from the group consisting of 2, 3 and 4. In certain embodiments, y of formula (Ib) is an integer selected from the group consisting of 2 and 3. In certain embodiments, y of formula (Ib) is an integer selected from the group consisting of 1, 2 and 3. In certain embodiments, y of formula (Ib) is 1. In certain embodiments, y of formula (Ib) is 2.

In certain embodiments, the CNP conjugate is of formula (Ia) with x=1.

In certain embodiments, -D of formula (Ia) or (Ib) has the sequence of SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25 or SEQ ID NO:30. In certain embodiments, -D of formula (Ia) or (Ib) has the sequence of SEQ ID NO:20, SEQ ID NO:21, SEQ ID NO:22, SEQ ID NO:23, SEQ ID NO:24 or SEQ ID NO:25.

In certain embodiments, -D of formula (Ia) or (Ib) has the sequence of SEQ ID NO:20. In certain embodiments, -D of formula (Ia) or (Ib) has the sequence of SEQ ID NO:21. In certain embodiments, -D of formula (Ia) or (Ib) has the sequence of SEQ ID NO:22. In certain embodiments, -D of formula (Ia) or (Ib) has the sequence of SEQ ID NO:23. In certain embodiments, -D of formula (Ia) or (Ib) has the sequence of SEQ ID NO:24. In certain embodiments, -D of formula (Ia) or (Ib) has the sequence of SEQ ID NO:25.

The moiety -L¹- of formula (Ia) or (Ib) is either conjugated to a functional group of the side chain of an amino acid residue of -D, to the N-terminal amine functional group or to the C-terminal carboxyl functional group of -D or to a nitrogen atom in the backbone polypeptide chain of -D. Attachment to either the N-terminus or C-terminus can either be direct through the corresponding amine or carboxyl functional group, respectively, or indirect wherein a spacer moiety is first conjugated to the amine or carboxyl functional group to which spacer moiety -L¹- is conjugated.

The moiety -L¹- of formula (Ia) or (Ib) is a reversible linker from which the drug, i.e. D-H is released in its free form, i.e. -L¹- is a traceless linker. Suitable reversible linkers are known in the art, such as for example the reversible linker moieties disclosed in WO 2005/099768 A2, WO 2006/136586 A2, WO 2011/089216 A1 and WO 2013/024053 A1, which are incorporated by reference herewith.

In certain embodiments, -L¹- is a reversible linker as described in WO 2011/012722 A1, WO 2011/089214 A1, WO 2011/089215 A1, WO 2013/024052 A1 and WO 2013/160340 A1 which are incorporated by reference herewith.

The moiety -L¹- can be connected to -D through any type of linkage, provided that it is reversible. In certain embodiments, -L¹- is connected to -D through a linkage selected from the group consisting of amide, ester, carbamate, acetal, aminal, imine, oxime, hydrazone, disulfide and acylguanidine. In certain embodiments, -L¹- is connected to -D through a linkage selected from the group consisting of amide, ester, carbamate and acylguanidine. It is understood that these linkages may not per se be reversible, but that neighboring groups comprised in -L¹- may render the linkage reversible.

In certain embodiments, the moiety -L¹- is connected to -D through an amide linkage.

A moiety -L¹- is disclosed in WO 2009/095479 A2. Accordingly, in certain embodiments, the moiety -L¹- is of formula (II):

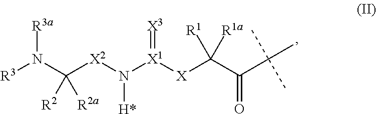

wherein the dashed line indicates the attachment to a nitrogen of -D which is a CNP moiety by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—;
X¹ is C; or S(O);
—X²— is —C(R⁸R^{8a})—; or —C(R⁸R^{8a})—C(R⁹R^{9a})—;
═X³ is =O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than —H they are connected to the N to which they are attached through an sp³-hybridized carbon atom;
—R⁷ is —N(R₁₀R^{10a}); or —NR¹⁰—(C═O)—R¹¹;
—R^{7a}, —R¹⁰, —R^{10a}, —R¹¹ are independently of each other —H; or C₁₋₆ alkyl;
optionally, one or more of the pairs —R^{1a}/—R^{4a}, —R^{1a}/R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
optionally, —R³/—R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²-Z and wherein -L¹- is optionally further substituted, provided that the hydrogen marked with the asterisk in formula (II) is not replaced by -L²-Z or a substituent;
   wherein
   -L²- is a single chemical bond or a spacer; and
   —Z is a water-soluble polymeric moiety;

In certain embodiments, -L¹- of formula (II) is substituted with one moiety -L²-Z. In certain embodiments, -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles, in which the atoms directly attached to the nitrogen are sp³-hybridized carbon atoms may be formed. In other words, such 3- to 10-membered heterocycle formed by —R³/—R^{3a} together with the nitrogen atom to which they are attached has the following structure:

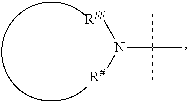

wherein
the dashed line indicates attachment to the rest of -L¹-;
the ring comprises 3 to 10 atoms comprising at least one nitrogen; and
R^{#} and R^{##} represent a sp³-hydridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

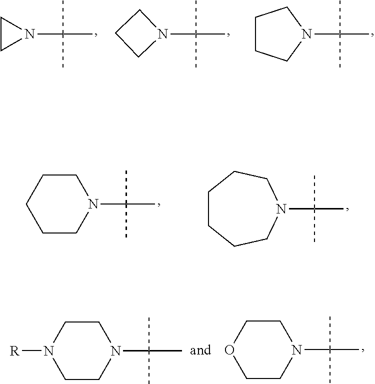

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

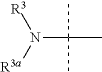

of formula (II) remains part of a primary, secondary or tertiary amine, i.e. —R³ and —R^{3a} are independently of each other —H or are connected to —N< through a sp³-hybridized carbon atom.

In certain embodiments, —R¹ or —R^{1a} of formula (II) is substituted with -L²-Z. In certain embodiments, —R² or —R^{2a} of formula (II) is substituted with -L²-Z. In certain embodiments, —R³ or —R^{3a} of formula (II) is substituted with -L²-Z. In certain embodiments, —R⁴ of formula (II) is substituted with -L²-Z. In certain embodiments, —R⁵ or —R^{5a} of formula (II) is substituted with -L²-Z. In certain embodiments, —R⁶ of formula (II) is substituted with -L²-Z. In certain embodiments, —R⁷ or —R^{7a} of formula (II) is substituted with -L²-Z. In certain embodiments, —R⁸ or —R^{8a} of formula (II) is substituted with -L²-Z. In certain embodiments, —R⁹ or —R^{9a} of formula (II) is substituted with -L²-Z.

In certain embodiments, —R⁴ of formula (II) is substituted with -L²-Z.

In certain embodiments, —X— of formula (II) is —C(R⁴R^{4a})— or —N(R⁴)—. In certain embodiments, —X— of formula (II) is —C(R⁴R^{4a})—.

In certain embodiments, X¹ of formula (II) is C.

In certain embodiments, ═X³ of formula (II) is ═O.

In certain embodiments, —X²— of formula (II) is —C(R⁸R^{8a})—.

In certain embodiments, —R⁸ and —R^{8a} of formula (II) are independently selected from the group consisting of —H, methyl and ethyl. In certain embodiments, at least one of —R⁸ and —R^{8a} of formula (II) is —H. In certain embodiments, both —R⁸ and —R^{8a} of formula (II) are —H.

In certain embodiments, —R¹ and —R^{1a} of formula (II) are independently selected from the group consisting of —H, methyl and ethyl. In certain embodiments, at least one of —R¹ and —R^{1a} of formula (II) is —H. In certain embodiments, both —R¹ and —R^{1a} of formula (II) are —H.

In certain embodiments, —R² and —R^{2a} of formula (II) are independently selected from the group consisting of —H, methyl and ethyl. In certain embodiments, at least one of —R² and —R^{2a} of formula (II) is —H. In certain embodiments, both —R² and —R^{2a} of formula (II) are H.

In certain embodiments, —R³ and —R^{3a} of formula (II) are independently selected from the group consisting of —H, methyl, ethyl, propyl and butyl. In certain embodiments, at least one of —R³ and —R^{3a} of formula (II) is methyl. In certain embodiments, —R³ and —R^{3a} of formula (II) are both —H. In certain embodiments, —R³ and —R^{3a} of formula (II) are both methyl. In certain embodiments, —R³ of formula (II) is —H and —R^{3a} of formula (II) is methyl.

In certain embodiments, —R⁴ and —R^{4a} of formula (II) are independently selected from the group consisting of —H, methyl and ethyl. In certain embodiments, at least one of —R⁴ and —R^{4a} of formula (II) is —H. In certain embodiments, both —R⁴ and —R^{4a} of formula (II) are —H.

In certain embodiments, the moiety -L¹- is of formula (IIa):

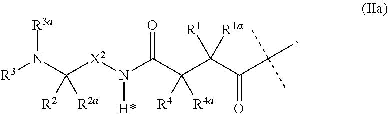

wherein the dashed line indicates the attachment to a nitrogen of -D which is a CNP moiety by forming an amide bond;
—R¹, —R^{1a}, —R², —R^{2a} R³, —R^{3a}, R⁴ —R^{4a} and —X²— are used as defined in formula (II); and wherein -L¹- is substituted with -L²-Z and wherein -L¹- is optionally further substituted, provided that the hydrogen marked with the asterisk in formula (IIa) is not replaced by -L²-Z or a substituent.

In certain embodiments, -L¹- of formula (IIa) is substituted with one moiety -L²-Z. In certain embodiments, the moiety -L¹- of formula (IIa) is not further substituted.

In certain embodiments, —R¹ and —R^{1a} of formula (IIa) are independently selected from the group consisting of —H, methyl and ethyl. In certain embodiments, at least one of —R¹ and —R^{1a} of formula (IIa) is —H. In certain embodiments, both —R¹ and —R^{1a} of formula (IIa) are —H.

In certain embodiments, —R⁴ and —R^{4a} of formula (IIa) are independently selected from the group consisting of —H, methyl and ethyl. In certain embodiments, at least one of —R⁴ and —R^{4a} of formula (IIa) is —H. In certain embodiments, both —R⁴ and —R^{4a} of formula (IIa) are —H.

In certain embodiments, —X²— of formula (IIa) is —C(R⁸R^{8a})—.

In certain embodiments, —R⁸ and —R^{8a} of formula (IIa) are independently selected from the group consisting of —H, methyl and ethyl. In certain embodiments, at least one of —R⁸ and —R^{8a} of formula (IIa) is —H. In certain embodiments, both —R⁸ and —R^{8a} of formula (IIa) are —H.

In certain embodiments, —R² and —R^{2a} of formula (IIa) are independently selected from the group consisting of —H, methyl and ethyl. In certain embodiments, at least one of —R² and —R^{2a} of formula (IIa) is —H. In certain embodiments, both —R² and —R^{2a} of formula (IIa) are H.

In certain embodiments, —R³ and —R^{3a} of formula (IIa) are independently selected from the group consisting of —H, methyl, ethyl, propyl and butyl. In certain embodiments, at least one of —R³ and —R^{3a} of formula (IIa) is methyl. In certain embodiments, —R³ and —R^{3a} of formula (IIa) are both —H. In certain embodiments, —R³ and —R^{3a} of formula (IIa) are both methyl. In certain embodiments, —R³ of formula (IIa) is —H and —R^{3a} of formula (IIa) is methyl.

In certain embodiments, the moiety -L¹- is of formula (IIb):

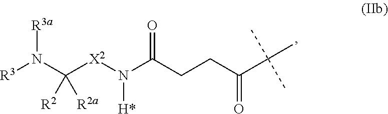

wherein the dashed line indicates the attachment to a nitrogen of -D which is a CNP moiety by forming an amide bond;
—R², —R^{2a}, —R³, —R^{3a} and —X²— are used as defined in formula (II); and
wherein -L¹- is substituted with -L²-Z and wherein -L¹- is optionally further substituted, provided that the hydrogen marked with the asterisk in formula (IIb) is not replaced by -L²-Z or a substituent.

In certain embodiments, -L¹- of formula (IIb) is substituted with one moiety -L²-Z. In certain embodiments, the moiety -L¹- of formula (IIb) is not further substituted.

In certain embodiments, —X²— of formula (IIb) is —C(R⁸R^{8a})—.

In certain embodiments, —R⁸ and —R^{8a} of formula (IIb) are independently selected from the group consisting of —H, methyl and ethyl. In certain embodiments, at least one of —R⁸ and —R^{8a} of formula (IIb) is —H. In certain embodiments, both —R⁸ and —R^{8a} of formula (IIb) are —H.

In certain embodiments, —R² and —R^{2a} of formula (IIb) are independently selected from the group consisting of —H, methyl and ethyl. In certain embodiments, at least one of —R² and —R^{2a} of formula (IIb) is —H. In certain embodiments, both —R² and —R^{2a} of formula (IIb) are H.

In certain embodiments, —R³ and —R^{3a} of formula (IIb) are independently selected from the group consisting of —H, methyl, ethyl, propyl and butyl. In certain embodiments, at least one of —R³ and —R^{3a} of formula (IIb) is methyl. In certain embodiments, —R³ and —R^{3a} of formula (IIb) are both —H. In certain embodiments, —R³ and —R^{3a} of formula (IIb) are both methyl. In certain embodiments, —R³ of formula (IIb) is —H and —R^{3a} of formula (IIb) is methyl.

In certain embodiments, the moiety -L¹- is of formula (IIb′):

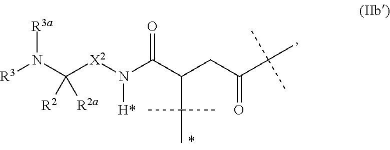

wherein
wherein the dashed line indicates the attachment to a nitrogen of -D which is a CNP moiety by forming an amide bond;
the dashed line marked with the asterisk indicates attachment to -L²-;
—R², —R^{2a}, —R³, —R^{3a} and —X²— are used as defined in formula (II); and
wherein -L¹- is optionally further substituted, provided that the hydrogen marked with the asterisk in formula (IIb′) is not replaced by a substituent.

In certain embodiments, the moiety -L¹- of formula (IIb′) is not further substituted.

In certain embodiments, —X²— of formula (IIb′) is —C(R⁸R^{8a})—.

In certain embodiments, —R⁸ and —R^{8a} of formula (IIb′) are independently selected from the group consisting of —H, methyl and ethyl. In certain embodiments, at least one of —R⁸ and —R^{8a} of formula (IIb′) is —H. In certain embodiments, both —R⁸ and —R^{8a} of formula (IIb′) are —H.

In certain embodiments, —R² and —R^{2a} of formula (IIb′) are independently selected from the group consisting of —H, methyl and ethyl. In certain embodiments, at least one of —R² and —R^{2a} of formula (IIb′) is —H. In certain embodiments, both —R² and —R^{2a} of formula (IIb′) are H.

In certain embodiments, —R³ and —R^{3a} of formula (IIb′) are independently selected from the group consisting of —H, methyl, ethyl, propyl and butyl. In certain embodiments, at least one of —R³ and —R^{3a} of formula (IIb′) is methyl. In certain embodiments, —R³ and —R^{3a} of formula (IIb′) are both —H. In certain embodiments, —R³ and —R^{3a} of formula (IIb′) are both methyl. In certain embodiments, —R³ of formula (IIb′) is —H and —R^{3a} of formula (IIb′) is methyl.

In certain embodiments, the moiety -L¹- is of formula (IIc):

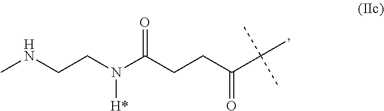

wherein the dashed line indicates the attachment to a nitrogen of -D which is a CNP moiety by forming an amide bond; and
wherein -L¹- is substituted with -L²-Z and wherein -L¹- is optionally further substituted, provided that the hydrogen marked with the asterisk in formula (IIc) is not replaced by -L²-Z or a substituent.

In certain embodiments, -L¹- of formula (IIc) is substituted with one moiety -L²-Z. In certain embodiments, the moiety -L¹- of formula (IIc) is not further substituted.

In certain embodiments, the moiety -L¹- is selected from the group consisting of formula (IIc-i), (IIc-ii), (IIc-iii), (IIc-iv) and (IIc-v):

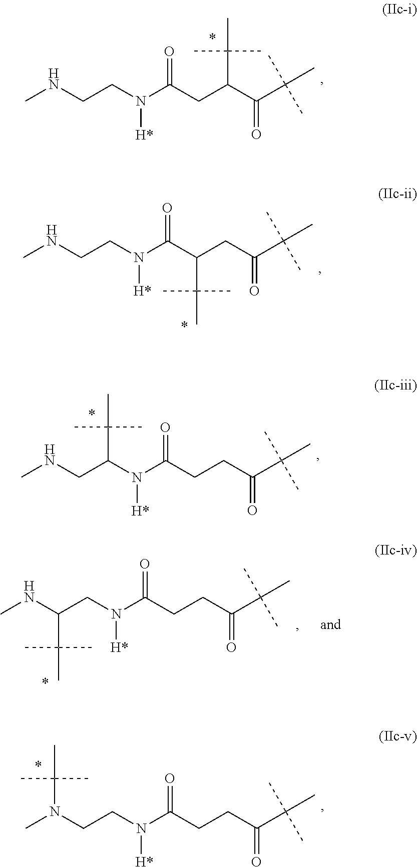

wherein
the unmarked dashed line indicates the attachment to a nitrogen of -D which is a CNP moiety by forming an amide bond; and
the dashed line marked with the asterisk indicates attachment to -L²-Z; and -L¹- is optionally further substituted, provided that the hydrogen marked with the asterisk in formula (IIc-i), (IIc-ii), (IIc-iii), (IIc-iv) and (IIc-v) is not replaced by a substituent.

In certain embodiments, the moiety -L¹- of formula (IIc-i), (IIc-ii), (IIc-iii), (IIc-iv) and (IIc-v) is not further substituted.

In certain embodiments, the moiety -L¹- is of formula (IIc-ii):

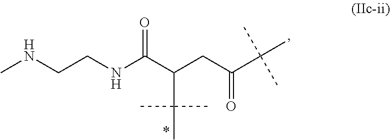

wherein
the unmarked dashed line indicates the attachment to a nitrogen of -D which is a CNP moiety by forming an amide bond; and
the dashed line marked with the asterisk indicates attachment to -L²-Z.

In certain embodiments, -L¹- of formula (IIc-ii) is substituted with one moiety -L²-Z.

The optional further substituents of -L¹- of formula (II), (IIa), (IIb), (IIb′), (IIc), (IIc-a), (IIc-b), (IIc-i), (IIc-ii), (IIc-iii), (IIc-iv), (IIc-v) are in certain embodiments as described above.

Another moiety -L¹- is disclosed in WO2016/020373A1. Accordingly, in certain embodiments, the moiety -L¹- is of formula (III):

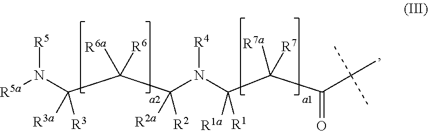

wherein
the dashed line indicates attachment to a primary or secondary amine or hydroxyl of -D which is a CNP moiety by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C—N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R”, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³—R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joined together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with -L²-Z and wherein -L¹- is optionally further substituted;
   wherein
   -L2- is a single chemical bond or a spacer; and
   —Z is a water-soluble polymeric moiety.

The optional further substituents of -L¹- of formula (III) are in certain embodiments as described above. In certain embodiments, -L¹- of formula (III) is substituted with one moiety -L²-Z. In certain embodiments, -L¹- of formula (III) is not further substituted.

Additional embodiments for -L¹- are disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

Additional embodiments for -L¹- are disclosed in U.S. Pat. No. 8,946,405B2 and U.S. Pat. No. 8,754,190B2, which are herewith incorporated by reference in their entirety. Accordingly, a moiety -L¹- is of formula (IV):

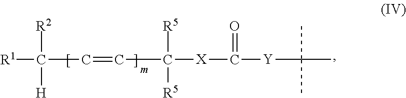

wherein
the dashed line indicates attachment to -D which is a CNP moiety and wherein attachment is through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with -L²-Z and wherein -L¹- is optionally further substituted;
   wherein
   -L2- is a single chemical bond or a spacer; and
   —Z is a water-soluble polymeric moiety.

The optional further substituents of -L¹- of formula (IV) are in certain embodiments, as described above. In certain embodiments, -L¹- of formula (IV) is substituted with one moiety -L²-Z. In certain embodiments, -L¹- of formula (IV) is not further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, in certain embodiments, 6 to 10 carbons, including groups such as phenyl, naphthyl, and anthracenyl. The term “heteroaryl” includes aromatic rings comprising 3 to 15 carbons containing at least one N, O or S atom, in certain embodiments, 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In certain embodiments, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂NR₂, wherein each R is independently alkyl, alkenyl, alkynyl, aryl or heteroaryl, or two R groups taken together with the atoms to which they are attached form a ring.

An additional embodiment for -L¹- is disclosed in WO2013/036857A1, which is herewith incorporated by reference in its entirety. Accordingly, in certain embodiments, moiety -L¹- is of formula (V):

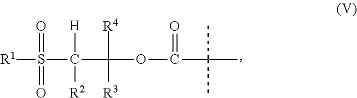

wherein
the dashed line indicates attachment to -D which is a CNP moiety and wherein attachment is through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵²;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with -L²-Z and wherein -L¹- is optionally further substituted;
   wherein
   -L2- is a single chemical bond or a spacer; and
   —Z is a water-soluble polymeric.

The optional further substituents of -L¹- of formula (V) are, in certain embodiments as described above.

In certain embodiments, -L¹- of formula (V) is substituted with one moiety -L²-Z.

In certain embodiments, -L¹- of formula (V) is not further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, in certain embodiments, 6-10 carbons, including groups such as phenyl, naphthyl, and anthracene “Heteroaryl” includes aromatic rings comprising 3-15 carbons containing at least one N, O or S atom, in certain embodiments, 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketone; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

Another embodiment for -L¹- is disclosed in WO 2022/115563 A1, which is herewith incorporated by reference in its entirety. Accordingly, in certain embodiments, -L¹- is of formula (Va):

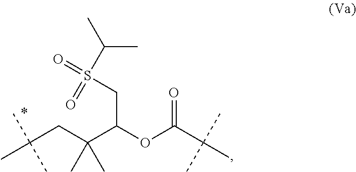

wherein the dashed line marked with the asterisk indicates the attachment to -L²-Z and the unmarked dashed line indicates the attachment to -D.

In certain embodiments, -L¹- is of formula (Va), the dashed line marked with the asterisk indicates the attachment to -L²-Z and the unmarked dashed line indicates the attachment to -D, wherein -D is a CNP moiety of the following amino acid sequence:

SEQ ID NO: 97 (CNP-38 N6Q, N14Q):
LQEHPQARKYKGAQKKGLSKGCFGLKLDRIGSMSGLGC,

wherein the cysteines at position 22 and 38 are connected through a disulfide-bridge; and wherein the attachment to -L¹- takes place either at the N-terminal or ring of the peptide.

Another embodiment for -L¹- is disclosed in U.S. Pat. No. 7,585,837B2, which is herewith incorporated by reference in its entirety. Accordingly, in certain embodiments, a moiety -L¹- is of formula (VI):

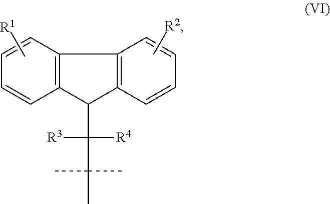

wherein
the dashed line indicates attachment to -D which is a CNP moiety and wherein attachment is through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with -L²-Z and wherein -L¹- is optionally further substituted;
   wherein
   -L2- is a single chemical bond or a spacer; and
   —Z is a water-soluble polymeric moiety.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

In certain embodiments, -L¹- of formula (VI) is substituted with one moiety -L²-Z. The optional further substituents of -L¹- of formula (VI) are in certain embodiments as described above.

In certain embodiments, -L¹- of formula (VI) is not further substituted.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, in certain embodiments, 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

A further embodiment for -L¹- is disclosed in WO2002/089789A1, which is herewith incorporated by reference in its entirety. Accordingly, a moiety -L¹- is of formula (VII):

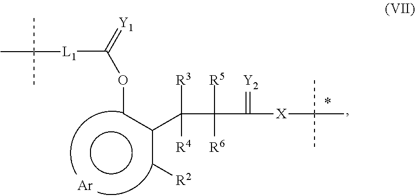

wherein
the dashed line indicates attachment to -D which is a CNP moiety and wherein attachment is through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃-1₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with -L²-Z and wherein -L¹- is optionally further substituted;
   wherein
   -L2- is a single chemical bond or a spacer; and
   —Z is a water-soluble polymeric moiety.

In certain embodiments, -L¹- of formula (VII) is substituted with one moiety -L²-Z. The optional further substituents of -L¹- of formula (VII) are in certain embodiments, as described above.

In certain embodiments, -L¹- of formula (VII) is not further substituted.

Only in the context of formula (VII) the terms used have the following meaning: The term “alkyl” shall be understood to include, e.g. straight, branched, substituted C₁₋₁₂ alkyls, including alkoxy, C₃—₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo- shall be understood to include fluoro, chloro, iodo and bromo.

In certain embodiments, -L¹- comprises a substructure of formula (VIII):

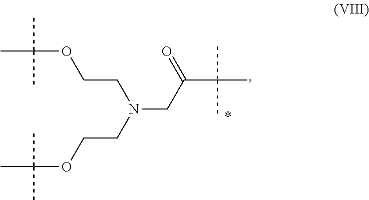

wherein
the dashed line marked with the asterisk indicates attachment to a nitrogen of -D which is a CNP moiety by forming an amide bond;
the unmarked dashed lines indicate attachment to the remainder of -L¹-; and
wherein -L¹- is substituted with -L²-Z and wherein -L¹- is optionally further substituted;
   wherein
   -L2- is a single chemical bond or a spacer; and
   —Z is a water-soluble polymeric moiety.

In certain embodiments, -L¹- of formula (VIII) is substituted with one moiety -L²-Z. The optional further substituents of -L¹- of formula (VIII) are as described above.

In certain embodiments, -L¹- of formula (VIII) is not further substituted.

In certain embodiments, -L¹- comprises a substructure of formula (IX):

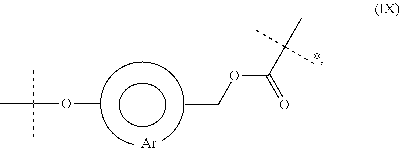

wherein
the dashed line marked with the asterisk indicates attachment to a nitrogen of -D which is a CNP moiety by forming a carbamate bond;
the unmarked dashed lines indicate attachment to the remainder of -L¹-; and
wherein -L¹- is substituted with -L²-Z and wherein -L¹- is optionally further substituted;
   wherein
   -L2- is a single chemical bond or a spacer; and
   —Z is a water-soluble polymeric moiety.

The optional further substituents of -L¹- of formula (IX) are as described above. In certain embodiments, -L¹- of formula (IX) is substituted with one moiety -L²-Z. In certain embodiments, -L¹- of formula (IX) is not further substituted.

The moiety -D may be connected to -L¹- through any functional group of D-H and is connected to -L¹- through an amine functional group of D-H. This may be the N-terminal amine functional group or an amine functional group provided by a lysine side chain, i.e. by the lysines at position 9, 11, 15, 16, 20 and 26, if the CNP has the sequence of SEQ ID NO:24.

Attachment of -L¹- to the ring of a CNP moiety significantly reduces the CNP conjugate's affinity to NPR-B compared to attachment at the N-terminus or to the non-ring part of CNP, which reduced affinity to NPR-B in turn reduces the risk of cardiovascular side effects, such as hypotension.

Accordingly, in certain embodiments, -L¹- is conjugated to the side chain of an amino acid residue of said ring moiety of -D or to the backbone of said ring moiety of -D. In certain embodiments, -L¹- is covalently and reversibly conjugated to the side chain of an amino acid residue of said ring moiety of -D. If -D is a CNP moiety with the sequence of SEQ ID NO:24, -L¹- is, in certain embodiments, conjugated to the amine functional group provided by the lysine at position 26 of the corresponding drug D-H.

The moiety -L²- is a chemical bond or a spacer moiety. In certain embodiments, -L²- is a chemical bond. In certain embodiments, -L²- is a spacer moiety.

The moiety -L²- can be attached to -L¹- by replacing any —H present, except where explicitly excluded.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R)C(O)N(R^{y1a})—, —OC(O)N(R^{y1})—, C₁₋₂₀ alkyl, C₂₋₂₀ alkenyl, and C₂₋₂₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

When -L²- is other than a single chemical bond, -L²- is selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments, -L²- has a molecular weight in the range of from 14 g/mol to 750 g/mol.

In certain embodiments, -L²- has a chain length of 1 to 20 atoms.

As used herein, the term “chain length” with regard to the moiety -L²- refers to the number of atoms of -L²- present in the shortest connection between -L¹- and —Z.

In certain embodiments, -L²- is of formula (i):

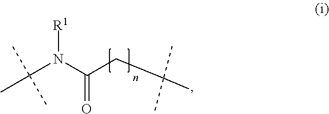

wherein
the dashed line marked with the asterisk indicates attachment to -L¹-;
the unmarked dashed line indicates attachment to —Z;
—R¹ is selected from the group consisting of —H, C₁₋₆ alkyl, C₂₋₆ alkenyl and C₂₋₆ alkynyl;
n is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments, —R¹ of formula (i) is selected from the group consisting of —H, methyl, ethyl, propyl, and butyl. In certain embodiments, —R¹ of formula (i) is selected from the group consisting of —H, methyl, ethyl and propyl. In certain embodiments, —R¹ of formula (i) is selected from the group consisting of —H and methyl. In certain embodiments, —R¹ of formula (i) is methyl.

In certain embodiments, n of formula (i) is selected from the group consisting of 0, 1, 2, 3, 4, 5, 6, 7, 8, 9 and 10. In certain embodiments, n of formula (i) is selected from the group consisting of 0, 1, 2, 3, 4 and 5. In certain embodiments, n of formula (i) is selected from the group consisting of 0, 1, 2 and 3. In certain embodiments, n of formula (i) is selected from the group consisting of 0 and 1. In certain embodiments, n of formula (i) is 0.

In certain embodiments, -L²- is a moiety selected from the group consisting of:

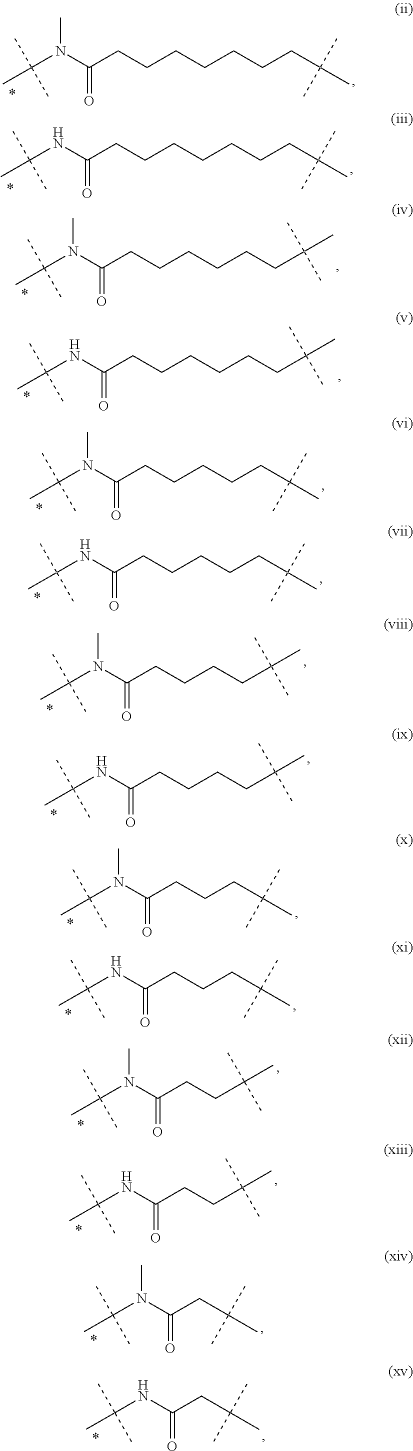

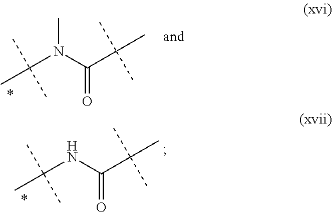

wherein
the dashed line marked with the asterisk indicates attachment to -L¹-;
the unmarked dashed line indicates attachment to —Z and
wherein the moieties (ii), (iii), (iv), (v), (vi), (vii), (viii), (ix), (x), (xi), (xii), (xiii), (xiv), (xv), (xvi) and (xvii) are optionally further substituted.

In certain embodiments, -L²- is selected from the group consisting of

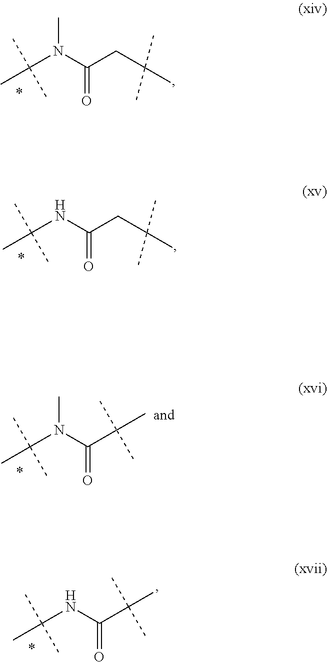

wherein
the dashed line marked with the asterisk indicates attachment to -L¹-; and
the unmarked dashed line indicates attachment to —Z.

In certain embodiments, -L²- is selected from the group consisting of

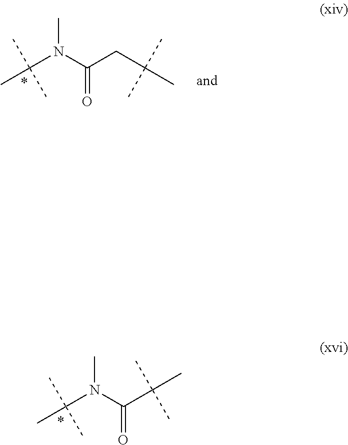

wherein
the dashed line marked with the asterisk indicates attachment to -L¹-; and
the unmarked dashed line indicates attachment to —Z.

In certain embodiments, -L²- is of formula (xvi):

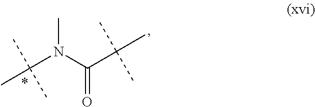

wherein
the dashed line marked with the asterisk indicates attachment to -L¹-; and
the unmarked dashed line indicates attachment to —Z.

In certain embodiments, the moiety -L¹-L²- is selected from the group consisting of

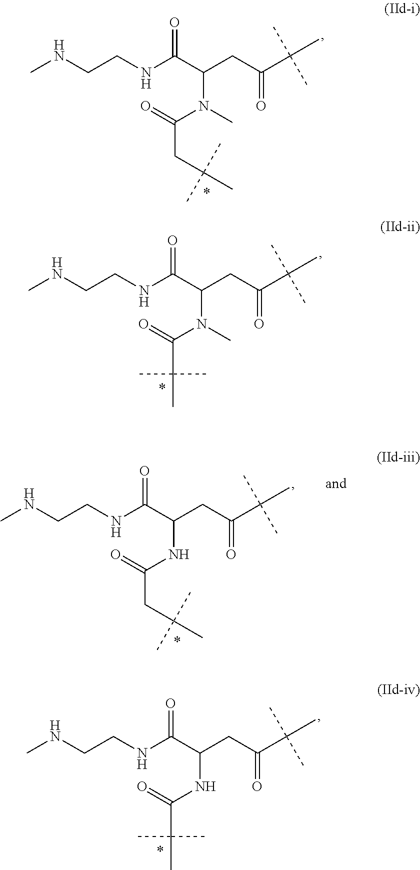

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-ii):

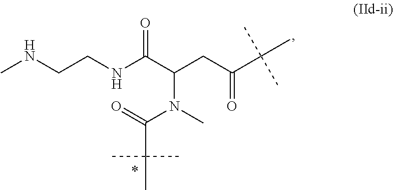

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is of formula (IId-ii′):

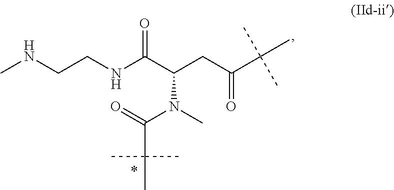

wherein
the unmarked dashed line indicates the attachment to a nitrogen of -D which is a CNP moiety by forming an amide bond; and
the dashed line marked with the asterisk indicates attachment to —Z.

In certain embodiments, the moiety -L¹-L²- is selected from the group consisting of

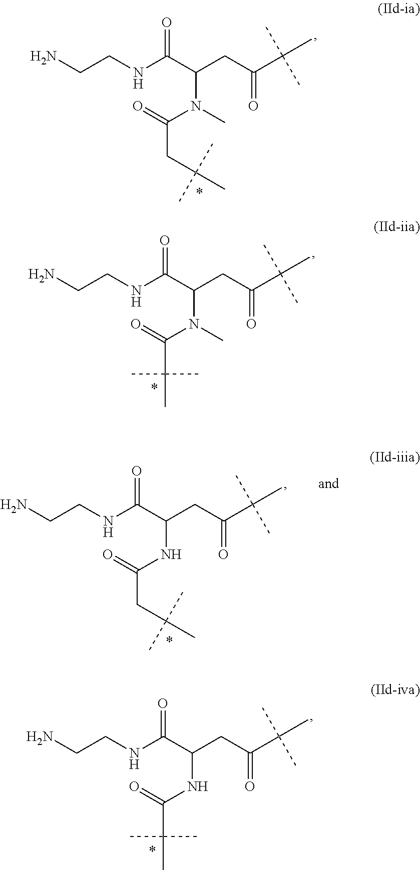

wherein
the unmarked dashed line indicates the attachment to a nitrogen of -D which is a CNP moiety by forming an amide bond; and
the dashed line marked with the asterisk indicates attachment to —Z.

In certain embodiments, —Z of formula (Ia) or (Ib) has a molecular weight ranging from 5 to 200 kDa. In certain embodiments, —Z of formula (Ia) or (Ib) has a molecular weight ranging from 8 to 100 kDa. In certain embodiments, —Z of formula (Ia) or (Ib) has a molecular weight ranging from 10 to 80 kDa. In certain embodiments, —Z of formula (Ia) or (Ib) has a molecular weight ranging from 12 to 60 kDa. In certain embodiments, —Z of formula (Ia) or (Ib) has a molecular weight ranging from 15 to 40 kDa. In certain embodiments, —Z of formula (Ia) or (Ib) has a molecular weight of about 20 kDa. In certain embodiments, —Z of formula (Ia) or (Ib) has a molecular weight of about 40 kDa.

The polymeric moiety —Z of formula (Ia) or (Ib) comprises a polymer. In certain embodiments, —Z of formula (Ia) or (Ib) comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, —Z of formula (Ia) or (Ib) comprises a protein. Preferred proteins are selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, —Z of formula (Ia) or (Ib) is apolysarcosine. In certain embodiments, —Z of formula (Ia) or (Ib) comprises poly(N-methylglycine). In certain embodiments, —Z of formula (Ia) or (Ib) comprises a random coil protein moiety. In certain embodiments, —Z of formula (Ia) or (Ib) comprises one random coil protein moiety. In certain embodiments, —Z of formula (Ia) or (Ib) comprises two random coil protein moieties. In certain embodiments, —Z of formula (Ia) or (Ib) comprises three random coil protein moieties. In certain embodiments, —Z of formula (Ia) or (Ib) comprises four random coil protein moieties. In certain embodiments, —Z of formula (Ia) or (Ib) comprises five random coil protein moieties. In certain embodiments, —Z of formula (Ia) or (Ib) comprises six random coil protein moieties. In certain embodiments, —Z of formula (Ia) or (Ib) comprises seven random coil protein moieties. In certain embodiments, —Z of formula (Ia) or (Ib) comprises eight random coil protein moieties.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, —Z of formula (Ia) or (Ib) comprises a fatty acid derivative. In certain embodiments, —Z of formula (Ia) or (Ib) is a fatty acid derivative. In certain embodiments, —Z of formula (Ia) is a fatty acid derivative and x is 1.

In certain embodiments, —Z of formula (Ia) or (Ib) is a fatty acid derivative as disclosed in WO 2006/097537 A2 which is herewith incorporated by reference.

In certain embodiments, —Z of formula (Ia) or (Ib) comprises a fatty acid derivative as disclosed in WO 2021/055497 A1 which is herewith incorporated by reference. Accordingly, in certain embodiments, —Z of formula (Ia) or (Ib) has the following structure (w):

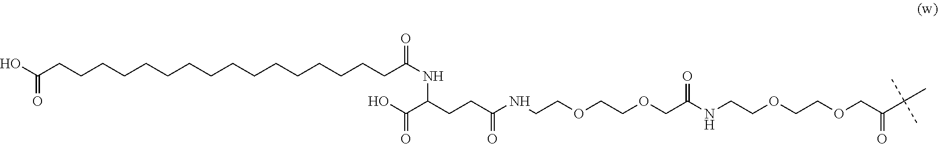

wherein the dashed line indicates the attachment to -L²- or -L¹- in formula (Ia) or (Ib).

In certain embodiments, —Z is of formula (w) and -L¹- is of formula (V).

In certain embodiments, —Z-L²-L¹- is of formula (w-a):

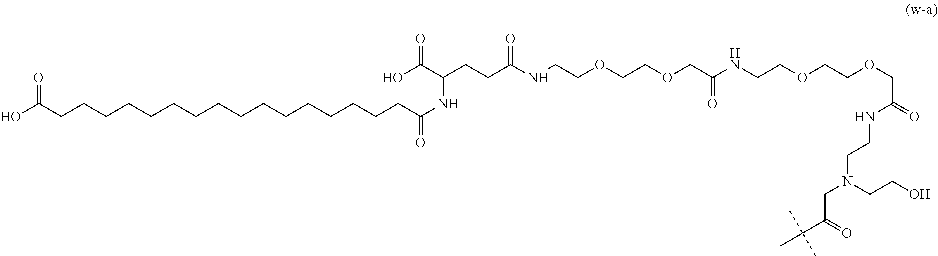

wherein the dashed line indicates the attachment to -D of formula (Ia) or (Ib).

In certain embodiments, CNP has the sequence selected from the group consisting of:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC.

In certain embodiments, CNP has the sequence selected from the group consisting of SEQ ID NO:98, SEQ ID NO:30, SEQ ID NO:99 and SEQ ID NO:100, —Z is of formula (w) and -L¹- is a reversible linker moiety. In certain embodiments, CNP has the sequence selected from the group consisting of SEQ ID NO:98, SEQ ID NO:30, SEQ ID NO:99 and SEQ ID NO:100, —Z is of formula (w) and -L¹- is of formula (V). In certain embodiments, CNP has the sequence selected from the group consisting of SEQ ID NO:98, SEQ ID NO:30, SEQ ID NO:99 and SEQ ID NO:100, —Z-L²-L¹- is of formula (w-a). Said -L¹- may be attached to said CNP via a lysine other than the lysine within the ring structure or it may be attached to the N-terminus.

In certain embodiments, CNP of SEQ ID NO:98, SEQ ID NO:30, SEQ ID NO:99 and SEQ ID NO:100 further comprises an acetyl group, such as an acetyl group at the N-terminus of the peptide. In certain embodiments, CNP of SEQ ID NO:98, SEQ ID NO:30, SEQ ID NO:99 and SEQ ID NO:100 further comprises an —OH or —NH₂ group at the C-terminus. In certain embodiments, CNP of SEQ ID NO:98, SEQ ID NO:30, SEQ ID NO:99 and SEQ ID NO:100 and -L¹- is attached to a residue of the CNP ring moiety or at a site other than the CNP moiety.

In certain embodiments, -L¹- is attached at a lysine residue, such as the lysine residue in bold in SEQ ID NO:98, SEQ ID NO:30, SEQ ID NO:99 and SEQ ID NO:100:

(SEQ ID NO: 98)
PGQEHPQARRYRGAQRRGLSRGCFGLKLDRIGSMSGLGC;
(SEQ ID NO: 30)
PGQEHPNARKYKGANKKGLSKGCFGLKLDRIGSMSGLGC;
(SEQ ID NO: 99)
PGQEHPNARRYRGANRRGLSRGCFGLKLDRIGSMSGLGC;
and
(SEQ ID NO: 100)
PGQEHPQARKYKGAQKKGLSKGCFGLKLDRIGSMSGLGC.

In certain embodiments, CNP is selected from the group consisting of:

(SEQ ID NO: 101)
Ac-PGQEHPQARRYRGAQRRGLSRGCFGLKLDRIGSMSGLGC;
(SEQ ID NO: 102)
Ac-PGQEHPNARKYKGANKKGLSKGCFGLKLDRIGSMSGLGC-NH₂;
(SEQ ID NO: 103)
Ac-PGQEHPNARRYRGANRRGLSRGCFGLKLDRIGSMSGLGC;
(SEQ ID NO: 104)
Ac-PGQEHPNARRYRGANRRGLSRGCFGLKLDRIGSMSGLGC-NH₂;
and
(SEQ ID NO: 105)
Ac-PGQEHPQARRYRGAQRRGLSRGCFGLKLDRIGSMSGLGC-NH₂.

In certain embodiments, —Z of formula (Ia) or (Ib) is a hyaluronic acid-based polymer.

In certain embodiments, —Z of formula (Ia) or (Ib) is a polymeric moiety as disclosed in WO 2013/024047 A1 which is herewith incorporated by reference.

In certain embodiments, —Z of formula (Ia) or (Ib) is a polymeric moiety as disclosed in WO 2013/024048 A1 which is herewith incorporated by reference.

In certain embodiments, —Z of formula (Ia) or (Ib) is a PEG-based polymer. In certain embodiments, —Z is a branched or multi-arm PEG-based polymer.

In certain embodiments, —Z of formula (Ia) or (Ib) is a branched polymer. In certain embodiments, —Z of formula (Ia) or (Ib) is a branched polymer having one, two, three, four, five or six branching points. In certain embodiments, —Z of formula (Ia) or (Ib) is a branched polymer having one, two or three branching points. In certain embodiments, —Z of formula (Ia) or (Ib) is a branched polymer having one branching point. In certain embodiments, —Z of formula (Ia) or (Ib) is a branched polymer having two branching points. In certain embodiments, —Z of formula (Ia) or (Ib) is a branched polymer having three branching points.

In certain embodiments, a branching point is selected from the group consisting of —N<, —CH< and >C<.

In certain embodiments, such branched moiety —Z of formula (Ia) or (Ib) is PEG-based.

In certain embodiments, such branched moiety —Z of formula (Ia) or (Ib) has a molecular weight ranging from and including 5 kDa to 500 kDa. In certain embodiments, such branched moiety —Z of formula (Ia) or (Ib) has a molecular weight ranging from and including 10 kDa to 250 kDa. In certain embodiments, such branched moiety —Z of formula (Ia) or (Ib) has a molecular weight ranging from and including 10 kDa to 150 kDa. In certain embodiments, such branched moiety —Z of formula (Ia) or (Ib) has a molecular weight ranging from and including 12 kDa to 100 kDa. In certain embodiments, such branched moiety —Z of formula (Ia) or (Ib) has a molecular weight ranging from and including 15 kDa to 80 kDa. In certain embodiments, such branched moiety —Z of formula (Ia) or (Ib) has a molecular weight ranging from and including 10 kDa to 80 kDa. In certain embodiments, the molecular weight is about 10 kDa. In certain embodiments, the molecular weight of such branched moiety —Z of formula (Ia) or (Ib) is about 20 kDa. In certain embodiments, the molecular weight of such branched moiety —Z of formula (Ia) or (Ib) is about 30 kDa. In certain embodiments, the molecular weight of such a branched moiety —Z of formula (Ia) or (Ib) is about 40 kDa. In certain embodiments, the molecular weight of such a branched moiety —Z of formula (Ia) or (Ib) is about 50 kDa. In certain embodiments, the molecular weight of such a branched moiety —Z of formula (Ia) or (Ib) is about 60 kDa. In certain embodiments, the molecular weight of such a branched moiety —Z of formula (Ia) or (Ib) is about 70 kDa. In certain embodiments, the molecular weight of such a branched moiety —Z of formula (Ia) or (Ib) is about 80 kDa. In certain embodiments, such branched moiety —Z of formula (Ia) or (Ib) has a molecular weight of about 40 kDa.

In certain embodiments, —Z comprises a moiety

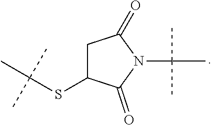

In certain embodiments, —Z comprises an amide bond.

In certain embodiments, —Z of formula (Ia) or (Ib) comprises a moiety of formula (a):

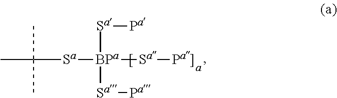

wherein
the dashed line indicates attachment to -L²- or to the remainder of —Z;
BP^{a} is a branching point selected from the group consisting of —N<, —CR< and >C<; —R is selected from the group consisting of —H and C₁₋₆ alkyl;
a is 0 if BP^{a} is —N< or —CR< and a is 1 if BP^{a} is >C<; —S^{a}—, —S^{a′}—, —S^{a″}— and —S^{a′″}— are independently of each other a chemical bond or are selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different; and
—P^{a′}, —P^{a″} and —P^{a′″} are independently a polymeric moiety.

Optionally, the moiety of formula (a) is substituted with one or more substituents.

In certain embodiments, BP^{a} of formula (a) is —N<. In certain embodiments, BP^{a} of formula (a) is —CR<. In certain embodiments, —R is —H.

Accordingly, in certain embodiments, a of formula (a) is 0.

In certain embodiments, BP^{a} of formula (a) is >C<.

In certain embodiments, —S^{a}— of formula (a) is a chemical bond.

In certain embodiments, —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a′}— of formula (a) is a chemical bond.

In certain embodiments, —S^{a}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—;
wherein —R⁴ and —R^{4a} are independently selected from the group consisting of —H, methyl, ethyl, propyl and butyl. In certain embodiments, —S^{a′}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a″}— of formula (a) is a chemical bond.

In certain embodiments, —S^{a″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a}”- of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —S^{a′″}— of formula (a) is a chemical bond.

In certain embodiments, —S^{a′″}— of formula (a) is selected from the group consisting of C₁₋₁₀ alkyl, C₂₋₁₀ alkenyl and C₂₋₁₀ alkynyl, which C₁₋₁₀ alkyl, C₂₋₁₀ alkenyl and C₂₋₁₀ alkynyl are optionally interrupted by one or more chemical groups selected from the group consisting of —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})—, and —OC(O)N(R⁴)—; wherein —R⁴ and —R^{4a} are independently selected from the group consisting of —H, methyl, ethyl, propyl and butyl. In certain embodiments, —S^{a′″}— of formula (a) is selected from the group consisting of methyl, ethyl, propyl, butyl, which are optionally interrupted by one or more chemical groups selected from the group consisting of —O—, —C(O)— and —C(O)N(R⁴)—.

In certain embodiments, —P^{a′}, —P^{a″} and —P^{a′″} of formula (a) independently comprise a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, —P^{a′}, —P^{a″} and —P^{a′″} of formula (a) independently have a molecular weight ranging from and including 5 kDa to 50 kDa, in certain embodiments ranging from and including 5 kDa to 40 kDa, in certain embodiments ranging from and including 7.5 kDa to 35 kDa, in certain embodiments ranging from and 7.5 to 30 kDa, in certain embodiments ranging from and including 10 to 30 kDa.

In certain embodiments, —P^{a′}, —P^{a″} and —P^{a′″} of formula (a) have a molecular weight of about 5 kDa. In certain embodiments, —P^{a′}, —P^{a″} and —P^{a′″} of formula (a) have a molecular weight of about 7.5 kDa. In certain embodiments, —P^{a′}, —P^{a″} and —P^{a′″} of formula (a) have a molecular weight of about 10 kDa. In certain embodiments, —P^{a′}, —P^{a″} and —P^{a′″} of formula (a) have a molecular weight of about 12.5 kDa. In certain embodiments, —P^{a′}, —P^{a″} and —P^{a′″} of formula (a) have a molecular weight of about 15 kDa. In certain embodiments, —P^{a′}, —P^{a″} and —P^{a′″} of formula (a) have a molecular weight of about 20 kDa.

In certain embodiments, —P^{a′}, —P^{a″} and —P^{a′″} of formula (a) independently comprise a PEG-based moiety. In certain embodiments, —P^{a′}, —P^{a″} and —P^{a′″} of formula (a) independently comprise a PEG-based moiety comprising at least 20% PEG, in certain embodiments at least 30% PEG, in certain embodiments at least 40% PEG, in certain embodiments at least 50% PEG, in certain embodiments at least 60% PEG, in certain embodiments at least 70% PEG, in certain embodiments at least 80% PEG and in certain embodiments at least 90% PEG.

In certain embodiments, —P^{a′}, —P^{a″} and —P^{a′″} of formula (a) independently comprise a protein moiety, in certain embodiments a random coil protein moiety and in certain embodiments a random coil protein moiety selected from the group consisting of PA, PAS, PAG, PG and XTEN moieties.

In certain embodiments, —P^{a′}, —P^{a″} and —P^{a′″} of formula (a) are a PA moiety. In certain embodiments, —P^{a′}, —P^{a″} and —P^{a′″} of formula (a) are a PAS moiety. In certain embodiments, —P^{a′}, —P^{a″} and —P^{a′″} of formula (a) are a PAG moiety. In certain embodiments, —P^{a′}, —P^{a″} and —P^{a′″} of formula (a) are a PG moiety. In certain embodiments, —P^{a′}, —P^{a″} and —P^{a′″} of formula (a) are an XTEN moiety.

In certain embodiments, —Z comprises one moiety of formula (a). In certain embodiments, —Z comprises two moieties of formula (a). In another embodiment, —Z comprises three moieties of formula (a). In certain embodiments, —Z comprises four moieties of formula (a). In certain embodiments, —Z comprises five moieties of formula (a). In certain embodiments, —Z comprises six moieties of formula (a).

In certain embodiments, —Z comprises a moiety of formula (b):

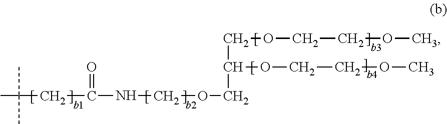

wherein
the dashed line indicates attachment to -L²- or to the remainder of —Z;
b1 is selected from the group consisting of 0, 1, 2, 3, 4, 5, 6, 7 and 8;
b2 is selected from the group consisting of 1, 2, 3, 4, 5, 6, 7 and 8;
b3 is an integer ranging from and including 150 to 1000; in certain embodiments, ranging from and including 150 to 500; and in certain embodiments, ranging from and including 200 to 460; and
b4 is an integer ranging from and including 150 to 1000; in certain embodiments, ranging from and including 150 to 500; and in certain embodiments, ranging from and including 200 to 460.

Optionally, the moiety of formula (b) is substituted with one or more substituents.

In certain embodiments, b3 and b4 of formula (b) are the same integer. In certain embodiments, b3 and b4 of formula (b) are both an integer ranging from 200 to 250 and in certain embodiments, b3 and b4 of formula (b) are about 225. In certain embodiments, b3 and b4 of formula (b) are both an integer ranging from 400 to 500 and in certain embodiments, b3 and b4 of formula (b) are about 450.

In certain embodiments, b1 of formula (b) is selected from the group consisting of 0, 1, 2, 3 and 4. In certain embodiments, b1 of formula (b) is selected from the group consisting of 1, 2 and 3. In certain embodiments, bi of formula (b) is 2.

In certain embodiments, b2 of formula (b) is selected from the group consisting of 1, 2, 3, 4 and 5. In certain embodiments, b2 of formula (b) is selected from the group consisting of 2, 3 and 4. In certain embodiments, b2 of formula (b) is 3.

In certain embodiments, b1 of formula (b) is 2, b2 of formula (b) is 3, and b3 and b4 are both about 450. In certain embodiments, b1 of formula (b) is 2, b2 of formula (b) is 3, and b3 and b4 are both about 225.

In certain embodiments, —Z comprises one moiety of formula (b). In certain embodiments, —Z comprises two moieties of formula (b). In certain embodiments, —Z comprises three moieties of formula (b). In certain embodiments, —Z comprises four moieties of formula (b). In certain embodiments, —Z comprises five moieties of formula (b). In certain embodiments, —Z comprises six moieties of formula (b).

In certain embodiments, —Z comprises a moiety of formula (c):

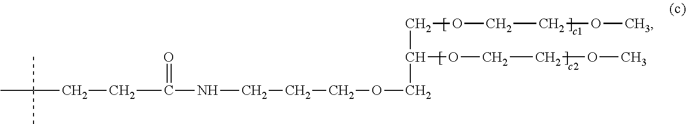

wherein
the dashed line indicates attachment to -L²- or to the remainder of —Z;
c1 and c2 are independently an integer ranging from and including 150 to 500; in certain embodiments, ranging from and including 200 to 460.

Optionally, the moiety of formula (c) is substituted with one or more substituents.

In certain embodiments, both c1 and c2 of formula (c) are the same integer.

In certain embodiments, c1 and c2 of formula (c) range from and include 200 to 250 and in certain embodiments, are about 225. In certain embodiments, c1 and c2 of formula (c) range from and include 400 to 500 and in certain embodiments, c1 and c2 of formula (c) are about 450.

In certain embodiments, the moiety —Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, —Z comprises one moiety of formula (c). In certain embodiments, —Z comprises two moieties of formula (c). In certain embodiments, —Z comprises three moieties of formula (c). In certain embodiments, —Z comprises four moieties of formula (c). In certain embodiments, —Z comprises five moieties of formula (c). In certain embodiments, —Z comprises six moieties of formula (c).

In certain embodiments, the moiety —Z is of formula (d):

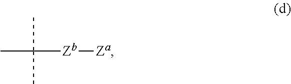

wherein
the dashed line indicates attachment to -L²-;
—Z^{b}— is selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each —R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁-6 alkyl is optionally substituted with one or more halogen, which are the same or different;
and
—Z^{a} is

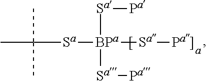

wherein
BP^{a}, —S^{a}—, —S^{a′}—, —S^{a″}—, —S^{a′″}—, —P^{a′}, —P^{a″}, —P^{a′″} and a are used as defined for formula (a).

Optionally, the moiety of formula (d) is substituted with one or more substituents.

In certain embodiments, BP^{a}, —S^{a′}—, —S^{a″}—, —S^{a′″}—, —P^{a′}, —P^{a″}, —P^{a′″} of formula (d) are as defined above for formula (a).

In certain embodiments, —Z^{a} of formula (d) is of formula (b). In certain embodiments, b1, b2, b3 and b4 are as described for formula (b).

In certain embodiments, the moiety —Z of formula (Ia) or (Ib) is of formula (e):

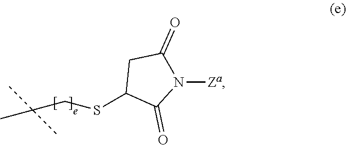

wherein
the dashed line indicates attachment to -L²-;
e is selected from the group consisting of 1, 2, 3, 4, 5, 6, 7, 8, 9, 10, 11, 12, 13, 14 and 15; and
—Z^{a} is

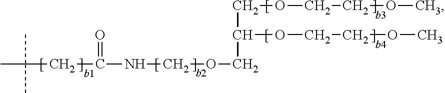

wherein
b1, b2, b3 and b4 are used as defined for formula (b).

Optionally, the moiety of formula (e) is substituted with one or more substituents.

In certain embodiments, for b1, b2, b3 and b4 of formula (e) are as defined above for formula (b).

In certain embodiments, e of formula (e) is 1. In certain embodiments, e of formula (e) is 2. In certain embodiments, e of formula (e) is 3. In certain embodiments, e of formula (e) is 4. In certain embodiments, e of formula (e) is 5. In certain embodiments, e of formula (e) is 6. In certain embodiments, e of formula (e) is 7. In certain embodiments, e of formula (e) is 8. In certain embodiments, e of formula (e) is 9. In certain embodiments, e of formula (e) is 10. In certain embodiments, e of formula (e) is 11. In certain embodiments, e of formula (e) is 12. In certain embodiments, e of formula (e) is 13. In certain embodiments, e of formula (e) is 14. In certain embodiments, e of formula (e) is 15.

In certain embodiments, e of formula (e) is selected from the group consisting of 2, 3, 4, 5, 6, 7, 8 and 9. In certain embodiments, e of formula (e) is selected from 3, 4, 5 and 6. In certain embodiments, e of formula (e) is 5. In certain embodiments, e of formula (e) is 5, b1 of formula (e) is 2, b2 of formula (e) is 3 and b3 and b4 of formula (e) are both about 450.

In certain embodiments, the moiety —Z of formula (Ia) or (Ib) is of formula (e-i) or (e-i′):

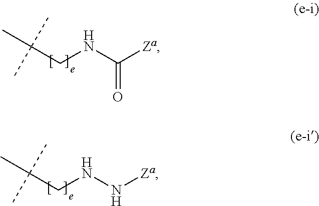

wherein
the dashed line indicates attachment to -L²-,
e is selected from the group consisting of 1, 2, 3, 4, 5, 6, 7, 8, 9, 10, 11, 12, 13, 14 and 15;
—Z^{a} is

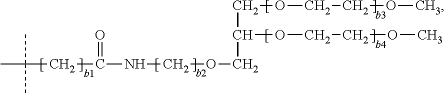

wherein
b1, b2, b3 and b4 are used as defined for formula (b).

In certain embodiments, for b1, b2, b3 and b4 of formula (e-i) and (e-i′) are as defined above for formula (b). In certain embodiments, e of formula (e-i) and (e-i′) are as described for formula (e). In certain embodiments, b1 of formula (e-i) and (e-i′) is 2, b2 of formula (e-i) and (e-i′) is 3 and b3 and b4 of formula (e-i) and (e-i′) are both about 450.

In certain embodiments, —Z of formula (Ia) or (Ib) is of formula (e-i).

In certain embodiments, the moiety —Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, the moiety —Z is of formula (f):

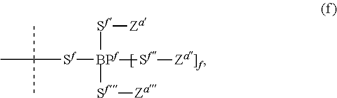

wherein
the dashed line indicates attachment to -L²-;
BP^{f}is a branching point selected from the group consisting of —N<, —CR< and >C<;
—R is selected from the group consisting of —H and C₁₋₆ alkyl;
f is 0 if BP^{f} is —N< or —CR< and f is 1 if BP^{f} is >C<;
—S^{f}—, —S^{f′}—, —S^{f″}— and —S^{f′″}— are independently either a chemical bond or are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each R¹ is independently selected from the group consisting of halogen, —CN, oxo (═O), —COOR³, —OR³, —C(O)R³, —C(O)N(R³R^{3a}), —S(O)₂N(R³R^{3a}, —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and
—Z^{a′}, —Z^{a″} and —Z^{a′″} are independently

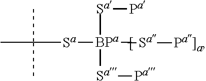

wherein
BP^{a}, —S^{a}—, —S^{a′}—, —S^{a″}—, —S^{a′″}—, —P^{a′}, —P^{a″}, —P^{a′″} and a are used as defined for formula (a).

Optionally, the moiety of formula (f) is substituted with one or more substituents.

In certain embodiments, BP^{a}, —S^{a}—, S^{a′}—, —S^{a″}—, —S^{a′″}—, —P^{a′}, —P^{a″}, —P^{a′″} of formula (f) are as defined above for formula (a).

In certain embodiments, BP^{f}of formula (f) is —CR< and r is 0. In certain embodiments, —R is —H.

In certain embodiments, —S^{f}— of formula (f) is a chemical bond.

In certain embodiments, —Z^{a′}, —Z^{a″} and —Z^{a′″} of formula (f) have the same structure. In certain embodiments, —Z^{a′}, —Z^{a″} and —Z^{a′″} of formula (f) are of formula (b).

In certain embodiments, b1, b2, b3 and b4 are as described for formula (b).

In certain embodiments, —S^{f}— of formula (f) is a chemical bond, BP^{a} of formula (f) is —CR< with —R being —H. In certain embodiments, —S^{f}— of formula (f) is a chemical bond, BP^{a} of formula (f) is —CR< with —R being —H and —Z^{a′}, —Z^{a″} and —Z^{a′″} of formula (f) are of formula (b).

In certain embodiments, —Z is of formula (g):

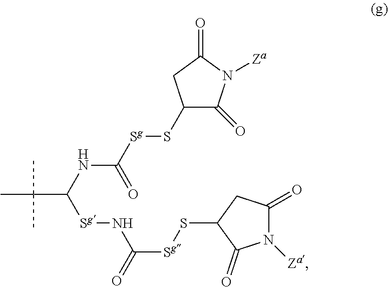

wherein
the dashed line indicates attachment to -L²-;
—S^{g}—, —S^{g′}— and —S^{g″}— are independently selected from the group consisting of C₁₋₅₀ alkyl, C₂₋₅₀ alkenyl, and C₂₋₅₀ alkynyl; wherein C₁₋₅₀ alkyl, C₂₋₅₀ alkenyl, and C₂₋₅₀ alkynyl are optionally substituted with one or more —R¹, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R²)—, —S(O)₂N(R²)—, —S(O)N(R²)—, —S(O)₂—, —S(O)—, —N(R²)S(O)₂N(R^{2a})—, —S—, —N(R²)—, —OC(OR²)(R^{2a})—, —N(R²)C(O)N(R^{2a})—, and —OC(O)N(R²)—;
   each -T- is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each -T- is independently optionally substituted with one or more —R¹, which are the same or different;
   each R¹ is independently selected from the group consisting of halogen, —CN, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different;
   each —R², —R^{2a}, —R³, —R^{3a} and —R^{3b} is independently selected from the group consisting of —H, and C₁₋₆ alkyl, wherein C₁₋₆ alkyl is optionally substituted with one or more halogen, which are the same or different;
and
—Z^{a} and —Z^{a} are independently

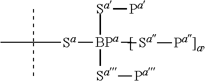

wherein
BP^{a}, —S^{a}—, —S^{a′}—, —S^{a″}—, —S^{a′″}—, —P^{a′}, —P^{a″}, —P^{a′″} and a are used as defined for formula (a).

Optionally, the moiety of formula (g) is substituted with one or more substituents.

In certain embodiments, BP^{a}, S^{a}—, —S^{a′}—, —S^{a″}—, —S^{a′″}—, —P^{a′}, —P^{a″} and —P^{a′″} of formula (g) are as defined above for formula (a).

In certain embodiments, —S^{g}— of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —N02, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl and butyl.

In certain embodiments, —S^{g}— of formula (g) is selected from C₁₋₆ alkyl.

In certain embodiments, —S^{g′}— of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl, and butyl.

In certain embodiments, —S^{g′}— of formula (g) is C₁₋₆ alkyl.

In certain embodiments, —S^{g}”- of formula (g) is selected from the group consisting of C₁₋₆ alkyl, C₂₋₆ alkenyl and C₂₋₆ alkynyl, which are optionally substituted with one or more —R¹, which is the same or different,
wherein
—R¹ is selected from the group consisting of halogen, oxo (═O), —COOR³, —OR³, —C(O)R³, —C(O)N(R³R^{3a}), —S(O)₂N(R³R^{3a}), —S(O)N(R³R^{3a}), —S(O)₂R³, —S(O)R³, —N(R³)S(O)₂N(R^{3a}R^{3b}), —SR³, —N(R³R^{3a}), —NO₂, —OC(O)R³, —N(R³)C(O)R^{3a}, —N(R³)S(O)₂R^{3a}, —N(R³)S(O)R^{3a}, —N(R³)C(O)OR^{3a}, —N(R³)C(O)N(R^{3a}R^{3b}), —OC(O)N(R³R^{3a}), and C₁₋₆ alkyl; wherein C₁₋₆ alkyl is optionally substituted with one or more halogen, which are the same or different; and —R³, —R^{3a} and —R^{3b} are independently selected from —H, methyl, ethyl, propyl and butyl.

In certain embodiments, —S^{g″}— of formula (g) is C₁₋₆ alkyl.

In certain embodiments, —Z^{a} and —Z^{a′} of formula (g) have the same structure. In certain embodiments, —Z^{a} and —Z^{a′} of formula (g) are of formula (b).

In certain embodiments, of BP^{a}, —S^{a}—, —S^{a′}—, —S^{a″}—, —S^{a′″}—, —P^{a′}, —P^{a″} and —P^{a′″} of formula (g-i) are as defined above for formula (a).

In certain embodiments, of —S^{g}—, —S^{g′}— and —S^{g″}— of formula (g-i) are as defined for formula (g).

In certain embodiments, —Z^{a} and —Z^{a} of formula (g-i) have the same structure. In certain embodiments, —Z^{a} and —Z^{a′} of formula (g-i) are of formula (b). In certain embodiments, for b1, b2, b3 and b4 are as described for formula (b).

In certain embodiments, —Z is of formula (h):

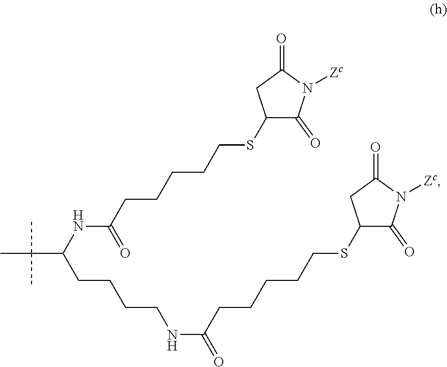

wherein
the dashed line indicates attachment to -L²-; and
each —Z^{c} is a moiety

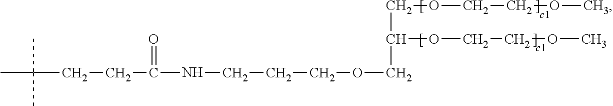

wherein
each c1 is an integer independently ranging from about 200 to 250.

Optionally, the moiety of formula (h) is substituted with one or more substituents.

In certain embodiments, both c1 of formula (h) are the same. In certain embodiments, both c1 of formula (h) are about 225.

In certain embodiments, the moiety —Z is of formula (h-i):

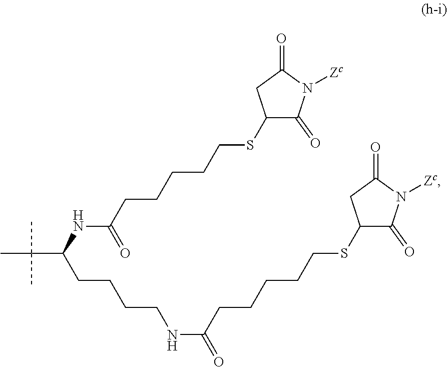

wherein
the dashed line indicates attachment to -L²-; and
each —Z^{c} is a moiety

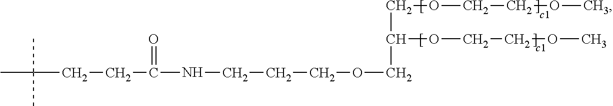

each c1 is an integer independently ranging from 200 to 250.

Optionally, the moiety of formula (h-i) is substituted with one or more substituents.

In certain embodiments, both cl of formula (h-i) are the same. In certain embodiments, both cl of formula (h-i) are about 225.

In certain embodiments, the CNP conjugate is of formula (IIf):

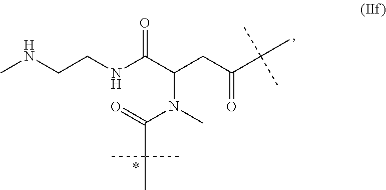

wherein
the unmarked dashed line indicates the attachment to a nitrogen of -D which is a CNP moiety by forming an amide bond; and
the dashed line marked with the asterisk indicates attachment to —Z having the structure

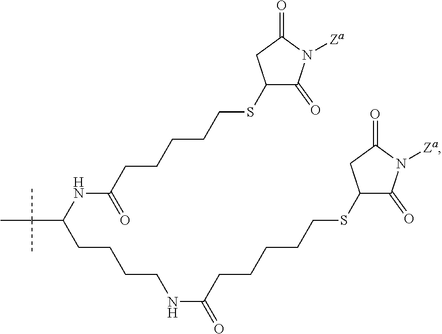

wherein
each —Z^{a} is

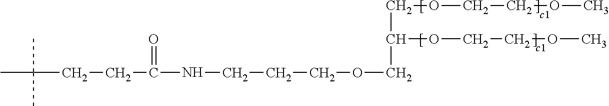

wherein
each c1 is an integer independently ranging from 200 to 250.

In certain embodiments, each c1 of formula (IIf) is about 225.

In certain embodiments, -D of formula (IIf) is a CNP moiety, i.e. the conjugate of formula (IIf) is a CNP conjugate. In certain embodiments, -D of formula (IIf) is a CNP moiety having the sequence of SEQ ID NO:24, SEQ ID NO:25 or SEQ ID NO:30. In certain embodiments, -D of formula (IIf) is a CNP moiety having the sequence of SEQ ID NO:24. In certain embodiments, -D of formula (IIf) is a CNP moiety having the sequence of SEQ ID NO:20. In certain embodiments, -D of formula (IIf) is a CNP moiety having the sequence of SEQ ID NO:21. In certain embodiments, -D of formula (IIf) is a CNP moiety having the sequence of SEQ ID NO:22. In certain embodiments, -D of formula (IIf) is a CNP moiety having the sequence of SEQ ID NO:23. In certain embodiments, -D of formula (IIf) is a CNP moiety having the sequence of SEQ ID NO:30.

In certain embodiments, -D of formula (IIf) is a CNP moiety which is attached to -L¹- through the nitrogen of the N-terminal amine functional group of CNP.

In certain embodiments, -D of formula (IIf) is a CNP moiety which is attached to -L¹- through a nitrogen provided by the amine functional group of a lysine side chain of the CNP moiety.

In certain embodiments, said lysine side chain is not part of the ring formed by the disulfide bridge between the cysteine residues at positions 22 and 38, if the CNP moiety is of SEQ ID NO:24.

Accordingly, in certain embodiments, the CNP moiety is connected to -L¹- in the CNP conjugate of formula (IIf) through the amine functional group provided by the side chain of the lysine at position 9, if the CNP has the sequence of SEQ ID NO:24.

In certain embodiments, the CNP moiety is connected to -L¹- in the CNP conjugate of formula (IIf) through the amine functional group provided by the side chain of the lysine at position 11, if the CNP has the sequence of SEQ ID NO:24.

In certain embodiments, the CNP moiety is connected to -L¹- in the CNP conjugate of formula (IIf) through the amine functional group provided by the side chain of the lysine at position 15, if the CNP has the sequence of SEQ ID NO:24.

In certain embodiments, the CNP moiety is connected to -L¹- in the CNP conjugate of formula (IIf) through the amine functional group provided by the side chain of the lysine at position 16, if the CNP has the sequence of SEQ ID NO:24.

In certain embodiments, the CNP moiety is connected to -L¹- in the CNP conjugate of formula (IIf) through the amine functional group provided by the side chain of the lysine at position 20, if the CNP has the sequence of SEQ ID NO:24.

In certain embodiments, said lysine side chain is part of the ring formed by the disulfide bridge between the cysteine residues at positions 22 and 38, if the CNP moiety is of SEQ ID NO:24.

Accordingly, in certain embodiments, the CNP moiety is connected to -L¹- in the CNP conjugate of formula (IIf) through the amine functional group provided by the side chain of the lysine at position 26, if the CNP has the sequence of SEQ ID NO:24.

In certain embodiments, the CNP conjugate is of formula (IIf), wherein cl is about 225, -D is a CNP moiety having the sequence of SEQ ID NO:20 and is attached to -L¹- through the amine functional group provided by the side chain of the lysine at position 30.

In certain embodiments, the CNP conjugate is of formula (IIf), wherein cl is about 225, -D is a CNP moiety having the sequence of SEQ ID NO:21 and is attached to -L¹- through the amine functional group provided by the side chain of the lysine at position 29.

In certain embodiments, the CNP conjugate is of formula (IIf), wherein cl is about 225, -D is a CNP moiety having the sequence of SEQ ID NO:22 and is attached to -L¹- through the amine functional group provided by the side chain of the lysine at position 28.

In certain embodiments, the CNP conjugate is of formula (IIf), wherein cl is about 225, -D is a CNP moiety having the sequence of SEQ ID NO:23 and is attached to -L¹- through the amine functional group provided by the side chain of the lysine at position 27.

In certain embodiments, the CNP conjugate is of formula (IIf), wherein cl is about 225, -D is a CNP moiety having the sequence of SEQ ID NO:30 and is attached to -L¹- through the amine functional group provided by the side chain of the lysine at position 27.

It is understood that the positions of the cysteines and lysines mentioned above vary depending on the lengths of the CNP moiety and that the person skilled in the art will have no difficulty identifying the corresponding cysteines and lysines in longer or shorter versions of the CNP moiety and also understands that for example some lysines may not be present in shorter CNP moieties. It is further understood that as a result of for example site-directed mutagenesis there might be more lysine residues in the non-ring forming part and/or ring forming part of the CNP moiety.

In certain embodiments, the CNP conjugate is of formula (IIf), wherein cl is about 225, -D is a CNP moiety having the sequence of SEQ ID NO:24 and is attached to -L¹- through the amine functional group provided by the side chain of the lysine at position 26.

In certain embodiments, the CNP conjugate is of formula (IIf′):

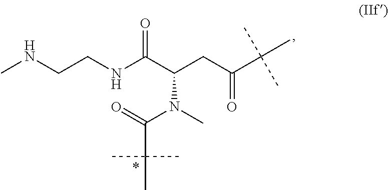

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

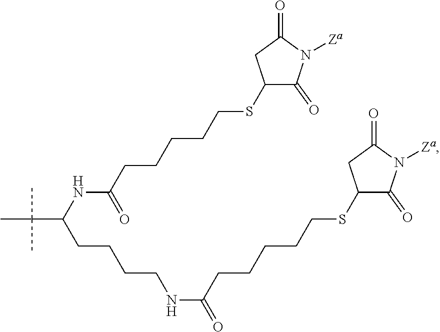

wherein
each Z^{a} is

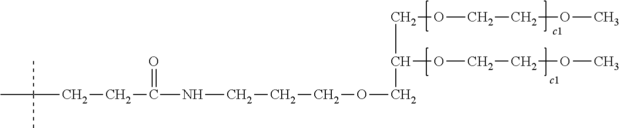

wherein
each c1 is an integer independently ranging from 200 to 250.

In certain embodiments, each c1 of formula (IIf′) is about 225.

In certain embodiments, the unit dosage form of the present invention comprises a CNP conjugate or pharmaceutically acceptable salt thereof of formula (IIf):

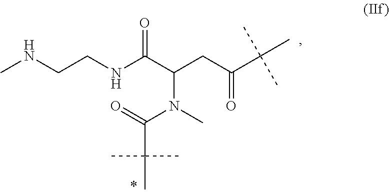

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

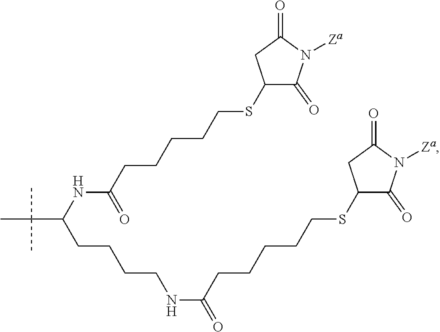

wherein
each Z^{a} is

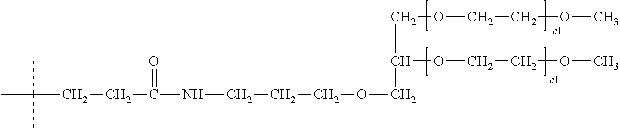

wherein
each c1 is an integer independently ranging from 200 to 250, and wherein the unit dose is about 50 μg CNP/kg.

In certain embodiments, the unit dosage form of the present invention comprises a CNP conjugate or pharmaceutically acceptable salt thereof of formula (IIf):

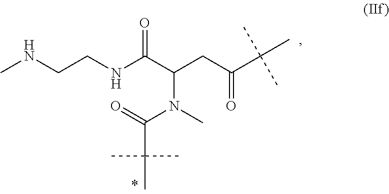

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

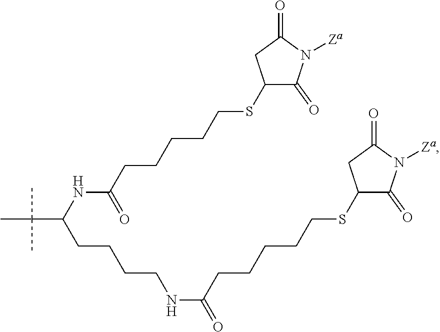

wherein
each Z^{a} is

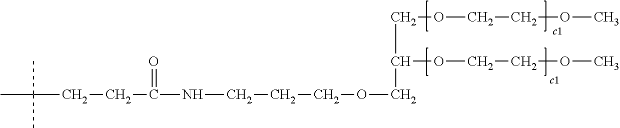

wherein
each c1 is an integer independently ranging from 200 to 250,
   and wherein the unit dose is about 75 μg CNP/kg.

In certain embodiments, the unit dosage form of the present invention comprises a CNP conjugate or pharmaceutically acceptable salt thereof of formula (IIf):

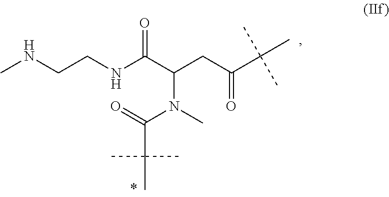

wherein
the unmarked dashed line indicates the attachment to a nitrogen provided by the side chain of the lysine at position 26 of a CNP moiety of SEQ ID NO:24 by forming an amide bond;
the dashed line marked with the asterisk indicates attachment to —Z having the structure

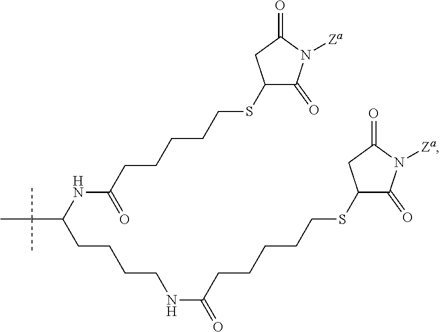

wherein
each Z^{c} is

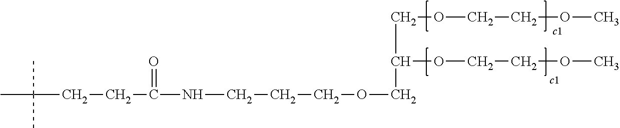

wherein
each c1 is an integer independently ranging from 200 to 250,
   and wherein the unit dose is about 100 μg CNP/kg.

In certain embodiments, the unit dosage form of the present invention comprises a CNP conjugate or pharmaceutically acceptable salt thereof of formula (IIf′):

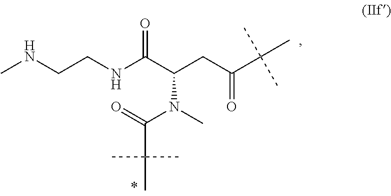

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

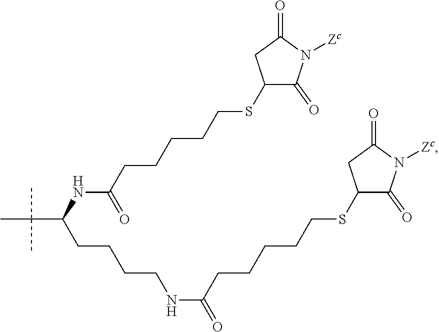

wherein
each Z^{c} is

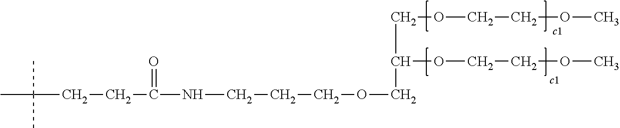

wherein
each c1 is an integer independently ranging from 200 to 250, and wherein the unit dose is 50 μg CNP/kg.

In certain embodiments, the unit dosage form of the present invention comprises a CNP conjugate or pharmaceutically acceptable salt thereof of formula (IIf′):

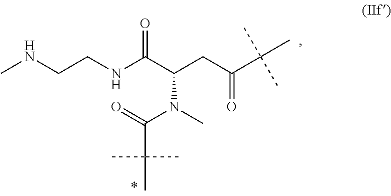

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

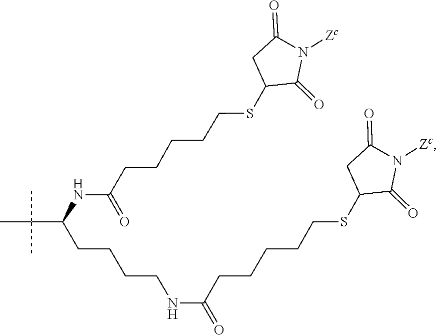

wherein
each Z^{a} is

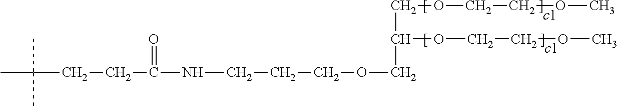

wherein
each c1 is an integer independently ranging from 200 to 250,
   and wherein the unit dose is 75 μg CNP/kg.

In certain embodiments, the unit dosage form of the present invention comprises a CNP conjugate or pharmaceutically acceptable salt thereof of formula (IIf′):

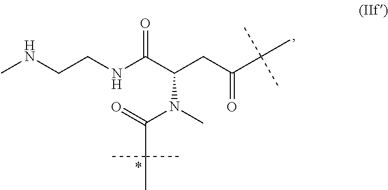

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

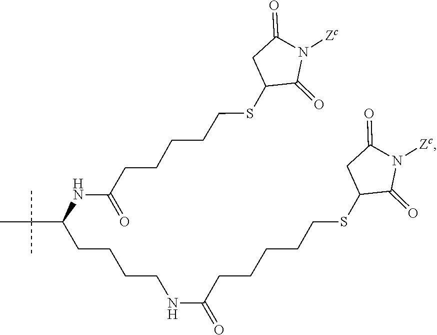

wherein
each Z^{a} is

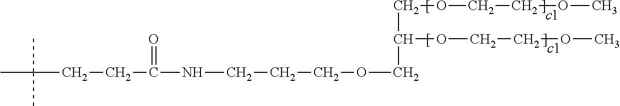

wherein
each c1 is an integer independently ranging from 200 to 250,
   and wherein the unit dose is 100 μg CNP/kg.

A further aspect of the present invention is a method of treating, reducing risk or delaying in a human patient a disease treatable by CNP, the method comprising the step of administering a CNP conjugate or pharmaceutically acceptable salt thereof at a unit dose from about 6 μg CNP/kg to at least about 150 μg CNP/kg.

In certain embodiments, the present invention is a method of treating, reducing risk or delaying in a human patient a disease treatable by CNP, the method comprising the step of administering a CNP conjugate or pharmaceutically acceptable salt thereof at a unit dose from about 6 μg CNP/kg to at least about 100 μg CNP/kg.

In certain embodiments, the present invention relates to a unit dosage form for use in the treatment of a disease treatable by CNP.

In certain embodiments, said disease treatable by CNP is selected from the group consisting of bone-related disorders such as skeletal dysplasias; cancer; autoimmune diseases; fibrotic diseases; inflammatory diseases; central nervous system diseases such as neurodegenerative diseases; infectious diseases; lung diseases; heart and vascular diseases; metabolic diseases and ophthalmic diseases.

In certain embodiments, said disease treatable by CNP is selected from the group consisting of achondroplasia, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, cardiovascular disease, neurological disease and obesity.

In certain embodiments, said disease treatable by CNP is selected from the group consisting of achondroplasia such as homozygous achondroplasia, hypochondroplasia, short stature, dwarfism, osteochondrodysplasias, thanatophoric dysplasia, osteogenesis imperfecta, achondrogenesis, chondrodysplasia punctata, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, obesity, disorders involving abnormal RAS-mitogen-activated protein kinase signaling, pulmonary hypertension, vasculopathy, endothelial dysfunction, liver cirrhosis, liver ascites, liver fibrosis, hepatorenal syndrome, asthma, pulmonary fibrosis, chronic kidney diseases, cardiorenal syndrome, dyspnea and lysosomal storage diseases such as mucopolysaccharidosis.

In certain embodiments, said disease treatable by CNP is one or more cardiovascular diseases selected from the group consisting of arrhythmia such as cardiac or sinus arrhythmia; atrial fibrillation; atrial flutter; bradycardia; Brugada syndrome; premature cardiac complexes; commotio cordis; heart block; long QT syndrome; parasystole; pre-excitation syndrome; tachycardia; ventricular fibrillation; ventricular flutter; cardiac conduction system disease; low cardiac output; cardiomegaly; dilated cardiomyopathy; hypertrophy such as left ventricular hypertrophy or right ventricular hyperthrophy; cardiomyopathy such as alcoholic, dilated, hypertrophic, restrictive, diabetic or Chagas cardiomyopathy; arrhythmogenic right ventricular dysplasia; endocardial fibroelastosis; endomyocardial fibrosis; glycogen storage disease type IIb; Keams-Sayre syndrome; myocardial reperfusion injury; myocarditis; sarcoglycanophaties; endocarditis such as bacterial or non-infective endocarditis; heart arrest; sudden cardiac death; out-of-hospital cardiat arrest; cardio-renal syndrome; paroxysmal dyspnea; cardiac edema, heart failure such as diastolic or systolic heart failure; heart valve disease; aortic valve insufficiency; aortic valve stenosis; heart valve prolapse; mitral valve insufficiency; mitral valve stenosis; pulmonary atresia; pulmonary valve insufficiency; pulmonary valve stenosis; tricuspid atresia; tricuspid valve insufficiency; tricuspid valve stenosis; myocardial ischemia; acute coronary syndrome; angina pectoris; coronary disease; Kounis syndrome; myocardial infarction; pulmonary heart disease; ventricular dysfunction such as left or right ventricular dysfunction; ventricular outflow obstruction; aortic valve stenosis, pulmonary valve stenosis; hypertension; atherosclerosis; restenosis; critical limb ischaemia; peripheral arterial disease; ischemia such as ischemia-reperfusion injury or ischemic injuries; abnormal fluid accumulation in the heart and myocardial edema.

In certain embodiments, said disease treatable by CNP is selected from the group consisting of ischemic heart disease such as myocardial infarction; congestive heart failure; arrhythmia and atherosclerosis.

In certain embodiments, said disease treatable by CNP is one or more central nervous system diseases selected from the group consisting of brain ischemia such as ischemic hypoxia; brain infarction; transient ischemic attack; vertebrobasilar insufficiency; cerebrovascular disorders; stroke; intracranial hemorrhages; corneal neovascularization; corneal transplantation; gragft-versus-host disease; graft rejection; glaucoma such as angle-closure, neovascular, open-angle or low tension glaucoma; ischemic optic neuropathy; central serous chorioretinopathy; retinopathy such as diabetic or hypertensive retinopathy; retinal degeneration; macular degeneration; geographic atrophy; macular edema; Stargardt disease; vitelliform macular dystrophy; wet macular degeneration; retinoschisis; retinal detachment; retinal perforations; retinal haemorrhage; retinal neovascularization; retinal vein occlusion; retinal artery occlusion; retinopathy of prematurity and proliferative vitreoretinopathy.

In certain embodiments, said disease treatable by CNP is selected from the group consisting of hypophosphatasia, hypochondroplasia, Muenke syndrome, hypertension, osteogenesis imperfecta and achondroplasia.

In certain embodiments, said disease treatable by CNP is selected from the group consisting of achondroplasia, hypochondroplasia, short stature, Noonan syndrome or SHOX deficiency.

In certain embodiments, said disease treatable by CNP is hypophosphatasia. In certain embodiments, said disease treatable by CNP is hypochondroplasia. In certain embodiments, said disease treatable by CNP is Muenke syndrome. In certain embodiments, said disease treatable by CNP is hypertension. In certain embodiments, said disease treatable by CNP is osteogenesis imperfecta. In certain embodiments, said disease treatable by CNP is achondroplasia.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of at least 24 nmol CNP/kg and is administered to a human patient, wherein said administration results in an annualized growth velocity (AGV) of about 5.4 cm/year, such as of about 5.42 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of about 24 nmol CNP/kg and is administered to a human patient, wherein said administration results in an annualized growth velocity (AGV) of 5.42 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of about 100 μg CNP/kg and is administered to a human patient, wherein said administration results in an annualized growth velocity (AGV) of about 5.4 cm/year, such as of about 5.42 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of 100 μg CNP/kg and is administered to a human patient, wherein said administration results in an annualized growth velocity (AGV) of 5.42 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 10 years, wherein said administration results in an annualized growth velocity (AGV) of 5.42 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 5 years, wherein said administration results in an annualized growth velocity (AGV) of about 5.95 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof of formula (IIf) or (IIf′) at a unit dose of 100 μg CNP/kg and is administered to a human patient aged 2 to 5 years, wherein said administration results in an annualized growth velocity (AGV) of 5.95 cm/year.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose ranging from 12.3 nmol CNP/kg to 36.9 nmol CNP/kg, wherein said unit dosage form is administered to a human patient with open epiphysis and said treatment reduces the incidence of achondroplasia-related adverse events in the human patient.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of 24.6 nmol CNP/kg, wherein said unit dosage form is administered to a human patient with open epiphysis and said treatment reduces the incidence of achondroplasia-related adverse events in the human patient.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of 100 μg CNP/kg, wherein said unit dosage form is administered to a human patient with open epiphysis and said treatment reduces the incidence of achondroplasia-related adverse events in the human patient.

In certain embodiments, the present invention relates to a unit dosage form for use in the treatment of skeletal dysplasia, such as achondroplasia, the unit dosage form comprising a CNP conjugate or pharmaceutically acceptable salt thereof of formula (IIf) or (IIf′) at a unit dose of 100 μg CNP/kg, wherein said unit dosage form is administered to a human patient with open epiphysis and said treatment reduces the incidence of achondroplasia-related adverse events in the human patient.

In certain embodiments, the present invention relates to a unit dosage form for use in the treatment of skeletal dysplasia, such as achondroplasia, the unit dosage form comprising a CNP conjugate or pharmaceutically acceptable salt thereof at a unit dose of 24.6 nmol CNP/kg, wherein said unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the present invention relates to a unit dosage form for use in the treatment of skeletal dysplasia, such as achondroplasia, the unit dosage form comprising a CNP conjugate or pharmaceutically acceptable salt thereof of formula (IIf) or (IIf′) at a unit dose of 100 μg CNP/kg, wherein said unit dosage form is administered weekly to a human patient with open epiphysis via subcutaneous injection and wherein each administration is associated with a frequency of injection site reaction of less than 3%, such as less than 2%, such as less than 1% or absence of injection site reaction.

A further aspect of the present invention is a method of treating achondroplasia in a human patient, the method comprising the step of administering a CNP conjugate or pharmaceutically acceptable salt thereof at a unit dose from about 6 μg CNP/kg to at least about 150 μg CNP/kg. Optionally, the unit dose is about 50 μg CNP/kg to about 150 μg CNP/kg, about 75 μg CNP/kg to about 125 μg CNP/kg, about 90 μg CNP/kg to about 110 μg CNP/kg or about 100 μg CNP/kg or 100 μg/CNP/kg. Such dosages are preferably administered weekly. Such dosages are preferably administered by weekly subcutaneous injection. A preferred compound in such methods is a compound of formula (IIf) or (IIf′) with dosages referring to the mass of the CNP1-38 moiety of said compound. Thus, a preferred regimen is a compound of formula (IIf) or (IIf′) administered subcutaneously weekly at unit doses of about 100 μg CNP/kg of the CNP1-38 moiety of the compound of formula (IIf) or (IIf′). Dosages of other compounds can be adjusted for equimolar delivery of the CNP moiety of the relevant compound as for the CNP moiety of a compound of formula (IIf) or (IIf′) within a tolerance of +/−20% or +/−10%.

Dosages regiments of other compounds or the compound of formula (IIf) or (IIf′) can be adjusted to deliver the same area under the curve +/−20% or +/−10% on a molar basis of CNP moiety, as a compound of formula (IIf) or (IIf′) administered weekly subcutaneously. The same regimens can be used in treating other disease treatable with CNP disclosed herein.

In certain embodiments, the present invention is a method of treating achondroplasia in a human patient, the method comprising the step of administering a CNP conjugate or pharmaceutically acceptable salt thereof at a unit dose of 6 μg CNP/kg, 20 μg CNP/kg, 50 μg CNP/kg, 75 μg CNP/kg, 100 μg CNP/kg or 125 μg CNP/kg.

In certain embodiments, the present invention is a method of treating achondroplasia in a human patient, the method comprising the step of administering a CNP conjugate or pharmaceutically acceptable salt thereof at a unit dose of 50 μg CNP/kg to 100 μg CNP/kg.

In certain embodiments, the present invention is a method of treating achondroplasia in a human patient, the method comprising the step of administering a CNP conjugate or pharmaceutically acceptable salt thereof at a unit dose of 50 μg CNP/kg.

In certain embodiments, the present invention is a method of treating achondroplasia in a human patient, the method comprising the step of administering a CNP conjugate or pharmaceutically acceptable salt thereof at a unit dose of 100 μg CNP/kg.

In certain embodiments, the present invention is a method of treating achondroplasia in a human patient, the method comprising the step of administering a CNP conjugate or pharmaceutically acceptable salt thereof at a unit dose of 50 μg CNP/kg to 100 μg CNP/kg, wherein the CNP conjugate is a compound of formula (IIf′):

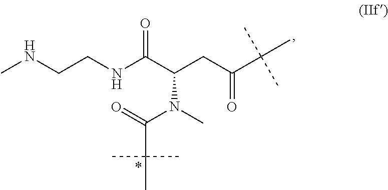

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

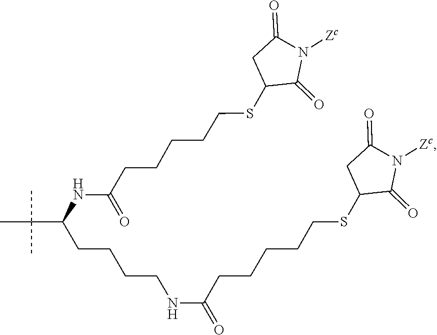

wherein
each Z^{c} is

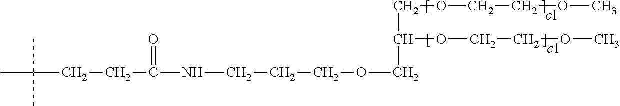

and wherein
each c1 is an integer independently ranging from 200 to 250.

In certain embodiments, the present invention is a method of treating achondroplasia in a human patient, the method comprising the step of administering a CNP conjugate or pharmaceutically acceptable salt thereof at a unit dose of 50 μg CNP/kg, wherein the CNP conjugate is a compound of formula (IIf′):

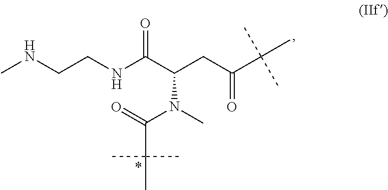

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

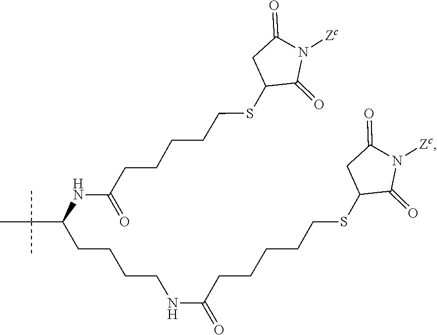

wherein
each Z^{c} is

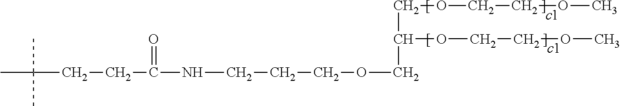

and wherein
each c1 is an integer independently ranging from 200 to 250.

In certain embodiments, the present invention is a method of treating achondroplasia in a human patient, the method comprising the step of administering a CNP conjugate or pharmaceutically acceptable salt thereof at a unit dose of 100 μg CNP/kg, wherein the CNP conjugate is a compound of formula (IIf′):

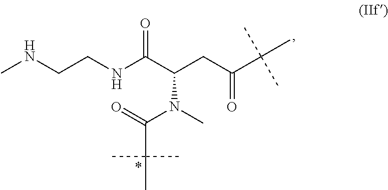

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

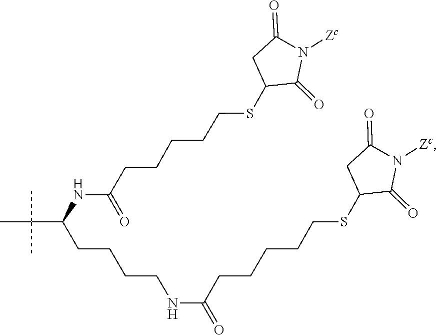

wherein
each Z^{c} is

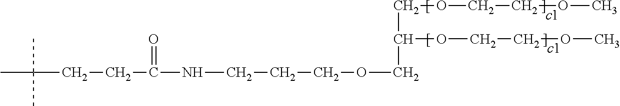

and wherein
each c1 is an integer independently ranging from 200 to 250.

In certain embodiments, the present invention is a method of treating achondroplasia in a human patient, the method comprising the step of administering a unit dosage comprising a unit dose of 24.6 nmol CNP/kg, wherein the unit dosage comprises a CNP conjugate or pharmaceutically acceptable salt thereof of formula (IIf) or (IIf′), wherein the age of the patient ranges from 2 to years old and said administration results in an annualized growth velocity of about 5.42 cm/year.

In certain embodiments, the present invention is a method of treating achondroplasia in a human patient, the method comprising the step of administering a unit dosage comprising a unit dose of 100 μg CNP/kg, wherein the unit dosage comprises a CNP conjugate or pharmaceutically acceptable salt thereof of formula (IIf) or (IIf′), wherein the age of the patient ranges from 2 to years old and said administration results in an annualized growth velocity of about 5.42 cm/year.

In certain embodiments, the present invention is a method of treating achondroplasia in a human patient, the method comprising the step of administering a unit dosage comprising a unit dose of 100 μg CNP/kg, wherein the unit dosage comprises a CNP conjugate or pharmaceutically acceptable salt thereof of formula (IIf) or (IIf′), wherein the age of the patient ranges from 2 to years old and said administration results in an annualized growth velocity of 5.42 cm/year.

In certain embodiments, the present invention is a method of treating achondroplasia in a human patient, the method comprising the step of administering a unit dosage comprising a unit dose of 100 μg CNP/kg, wherein the unit dosage comprises a CNP conjugate or pharmaceutically acceptable salt thereof of formula (IIf) or (IIf′), wherein the age of the patient ranges from 2 to years old and said administration results in an annualized growth velocity of about 5.95 cm/year.

In certain embodiments, the present invention is a method of treating achondroplasia in a human patient, the method comprising the step of administering a unit dosage comprising a unit dose of 100 μg CNP/kg, wherein the unit dosage comprises a CNP conjugate or pharmaceutically acceptable salt thereof of formula (IIf) or (IIf′), wherein the age of the patient ranges from 2 to years old and said administration results in an annualized growth velocity of 5.95 cm/year.

Unit doses may be administered once or multiple times. For multiple administrations, the interval is preferably weekly, but can be twice a week, biweekly, or monthly among others. In certain embodiments, one unit dose is administered weekly via one subcutaneous injection. In certain embodiments, one unit dose is administered monthly via one subcutaneous injection. In certain embodiments, for patient groups weighing 55 kg or above, one unit dose may be split and administered via two simultaneous or successive injections.

The CNP conjugate or pharmaceutically acceptable salt thereof may be administered for at least six months, a year, five years, ten years, until a patient is 18 years old, until patient's epiphyseal closure or indefinitely. In certain embodiments, the CNP conjugate or pharmaceutically acceptable salt thereof may be administered until a patient is 18 years old. In certain embodiments, the CNP conjugate or pharmaceutically acceptable salt thereof may be administered until a patient's ephiphysis is closed.

Treatment may start antenatally, at birth or on diagnosis of a deficit or risk relating to CNP.

If a unit dose is determined for a particular drug, such as as a reversible CNP conjugate of formula (IIf) or (IIf′), then the unit dose can be used as a guide for other reversible CNP conjugates, such that the unit dose of other reversible conjugates is the same by moles of CNP as that for CNP of formula (IIf) or (IIf′). Such guidance is particularly useful when the other conjugate releases a CNP moiety with a release half-life within plus or minus 20% of that of the conjugate of CNP of formula (IIf) or (IIf′). Thus, for example, if a unit dose is determined for a reversible conjugate of CNP-38, then an otherwise similar reversible conjugate of CNP-53 can be administered by multiplying the dose for CNP-38 by the ratio of molecular weights of CNP-53/CNP-38.

In certain embodiments, the present invention is a method of increasing growth velocity in a human patient, the method comprising the step of administering a CNP conjugate or pharmaceutically acceptable salt thereof at a unit dose from about 6 μg CNP/kg to at least about 150 μg CNP/kg.

In certain embodiments, the present invention is a method of increasing growth velocity in a human patient, the method comprising the step of administering a CNP conjugate or pharmaceutically acceptable salt thereof at a unit dose of 6 μg CNP/kg, 20 μg CNP/kg, 50 μg CNP/kg, 75 μg CNP/kg, 100 μg CNP/kg or 125 μg CNP/kg.

In certain embodiments, the present invention is a method of increasing growth velocity in a human patient, the method comprising the step of administering a CNP conjugate or pharmaceutically acceptable salt thereof at a unit dose of 50 μg CNP/kg to 100 μg CNP/kg.

In certain embodiments, the present invention is a method of increasing growth velocity in a human patient, the method comprising the step of administering a CNP conjugate or pharmaceutically acceptable salt thereof at a unit dose of 50 μg CNP/kg.

In certain embodiments, the present invention is a method of increasing growth velocity in a human patient, the method comprising the step of administering a CNP conjugate or pharmaceutically acceptable salt thereof at a unit dose of 100 μg CNP/kg.

In certain embodiments, the present invention is a method of increasing growth velocity in a human patient, the method comprising the step of administering a CNP conjugate or pharmaceutically acceptable salt thereof at a unit dose ranging from about 12.3 nmol CNP/kg to about 36.9 nmol CNP/kg for a period of at least 52 weeks, said administration resulting in an annualized growth velocity (AGV) of about 5.4 cm/year.

In certain embodiments, the present invention is a method of increasing growth velocity in a human patient, the method comprising the step of administering a CNP conjugate or pharmaceutically acceptable salt thereof at a unit dose of at least 24 nmol CNP/kg for a period of at least 52 weeks, said administration resulting in an annualized growth velocity (AGV) of about 5.4 cm/year.

In certain embodiments, the present invention is a method of increasing growth velocity in a human patient, the method comprising the step of administering a CNP conjugate or pharmaceutically acceptable salt thereof at a unit dose of about 24.6 nmol CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof at a unit dose of 24.6 nmol CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof at a unit dose of 24.6 nmol CNP/kg for a period of at least 52 weeks to until the patient's ephiphysis is closed, said patient being 2 to 10 years of age, said administration resulting in an annualized growth velocity (AGV) of 5.42 cm/year.

In certain embodiments, the present invention is a method of increasing growth velocity in a human patient, the method comprising the step of administering a CNP conjugate or pharmaceutically acceptable salt thereof at a unit dose of 24.6 nmol CNP/kg for a period of at least 52 weeks to until the patient's ephiphysis is closed, said patient being 2 to 5 years of age, said administration resulting in an annualized growth velocity (AGV) of about 5.95 cm/year, such as of 5.95 cm/year.

In certain embodiments, the present invention is a method of increasing growth velocity in a human patient, the method comprising the step of administering a CNP conjugate or pharmaceutically acceptable salt thereof at a unit dose of 24.6 nmol CNP/kg for a period of at least 104 weeks, said administration resulting in an annualized growth velocity (AGV) of about 5.42 cm/year.

The annualized growth velocity can be determined by either measuring the increase in a patient's height for at least 52 weeks, such as for at least 104 weeks or 156 weeks or by measuring for a fraction of one year, such as for 3, 4, 5, 6, 7, 8, 9, 10, 11 or 12 months and then extrapolating the value to one full year.

In certain embodiments, the present invention is a method of increasing growth velocity in a human patient, the method comprising the steps of:
(a) administering to a human patient of a CNP conjugate or pharmaceutically acceptable salt thereof at a unit dose of 24.6 nmol CNP/kg for a period of at least 52 weeks;
(b) measuring the absolute height at the time points of 0 months, 6 months and 12 months; and
(c) calculating the AGV between the period of 0 to 6 months and the period of 6 to 12 months.

In certain embodiments, the present invention is a method of increasing growth velocity in a human patient, the method comprising the step of administering a CNP conjugate or pharmaceutically acceptable salt thereof at a unit dose of at least 100 μg CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof at a unit dose of about 100 μg CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof at a unit dose of 100 μg CNP/kg for a period of at least 52 weeks, said administration resulting in an annualized growth velocity (AGV) of 5.42 cm/year.

In certain embodiments, the present invention is a method of increasing growth velocity in a human patient, the method comprising the step of administering a CNP conjugate or pharmaceutically acceptable salt thereof at a unit dose of 100 μg CNP/kg for a period of at least 52 weeks until the patient's ephiphysis is closed, said patient being 2 to 10 years of age, said administration resulting in an annualized growth velocity (AGV) of 5.42 cm/year.

In certain embodiments, the present invention is a method of increasing growth velocity in a human patient, the method comprising the step of administering a CNP conjugate or pharmaceutically acceptable salt thereof at a unit dose of 100 μg CNP/kg for a period of at least 52 weeks until the patient's ephiphysis is closed, said patient being 2 to 5 years of age, said administration resulting in an annualized growth velocity (AGV) of about 5.95 cm/year, such as 5.95 cm/year.

In certain embodiments, the present invention is a method of increasing growth velocity in a human patient, the method comprising the step of administering a CNP conjugate or pharmaceutically acceptable salt thereof at a unit dose of 100 μg CNP/kg for a period of at least 104 weeks, said administration resulting in an annualized growth velocity (AGV) of 5.42 cm/year.

In certain embodiments, the present invention is a method of increasing growth velocity in a human patient, the method comprising the step of administering a CNP conjugate or pharmaceutically acceptable salt thereof at a unit dose ranging from 12.3 nmol CNP/kg to 36.9 nmol CNP/kg for a period of at least 52 weeks, wherein said CNP conjugate unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the present invention is a method of increasing growth velocity in a human patient, the method comprising the step of administering a CNP conjugate or pharmaceutically acceptable salt thereof at a unit dose of 24.6 nmol CNP/kg for a period of at least 52 weeks, wherein said CNP conjugate or unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the present invention is a method of increasing growth velocity in a human patient, the method comprising the step of administering a CNP conjugate or pharmaceutically acceptable salt thereof at a unit dose of 100 μg CNP/kg for a period of at least 52 weeks, wherein said CNP conjugate or unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 52 weeks, wherein said CNP conjugate or unit dosage form is administered weekly to a human patient with open epiphysis via subcutaneous injection and wherein each administration is associated with a frequency of injection site reaction of less than 3%, such as less than 2%, such as less than 1% or absence of injection site reaction.

In certain embodiments, the frequency of injection site reaction is less than 3%. In certain embodiments, the frequency of injection site reaction is less than 2%. In certain embodiments, the frequency of injection site reaction is less than 1%. In certain embodiments, there are no injection site reactions.

In certain embodiments, the present invention is a method of increasing growth velocity in a achondroplasia human patient, the method comprising the step of administering a CNP conjugate or pharmaceutically acceptable salt thereof at a unit dose ranging from 12.3 nmol CNP/kg to 36.9 nmol CNP/kg for a period of at least 52 weeks, wherein said CNP conjugate or unit dosage form is administered to a human patient with open epiphysis and said treatment reduces the incidence of achondroplasia-related adverse events in the human patient.

In certain embodiments, the present invention is a method of increasing growth velocity in a achondroplasia human patient, the method comprising the step of administering a CNP conjugate or pharmaceutically acceptable salt thereof at a unit dose of 24.6 nmol CNP/kg for a period of at least 52 weeks, wherein said CNP conjugate or unit dosage form is administered to a human patient with open epiphysis and said treatment reduces the incidence of achondroplasia-related adverse events in the human patient.

In certain embodiments, the present invention is a method of increasing growth velocity in a achondroplasia human patient, the method comprising the step of administering a CNP conjugate or pharmaceutically acceptable salt thereof of formula (IIf) or (IIf′) at a unit dose of 100 μg CNP/kg for a period of at least 52 weeks, wherein said CNP conjugate or unit dosage form is administered to a human patient with open epiphysis and said treatment reduces the incidence of achondroplasia-related adverse events in the human patient.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 50 μg CNP/kg to 100 μg CNP/kg.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 50 μg CNP/kg.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg.

In certain embodiments, the present invention is a method of increasing growth velocity in a human patient, the method comprising the step of administering a CNP conjugate or pharmaceutically acceptable salt thereof of formula (IIf) or (IIf′) at a unit dose of at least 100 μg CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of about 100 μg CNP/kg for a period of at least 52 weeks, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 52 weeks, said administration resulting in an annualized growth velocity (AGV) of 5.42 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 52 weeks until the patient's ephiphysis is closed, said patient being 2 to 10 years of age, said administration resulting in an annualized growth velocity (AGV) of about 5.42 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 52 weeks until the patient's ephiphysis is closed, said patient being 2 to 5 years of age, said administration resulting in an annualized growth velocity (AGV) of about 5.95 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 104 weeks, said administration resulting in an annualized growth velocity (AGV) of at least about 5.42 cm/year.

In certain embodiments, the present invention is a method of increasing growth velocity in a human patient, the method comprising the step of administering a CNP conjugate or pharmaceutically acceptable salt thereof of formula (IIf) or (IIf′) at a unit dose of 100 μg CNP/kg for a period of at least 104 weeks, said administration resulting in an annualized growth velocity (AGV) of about 5.42 cm/year.

In certain embodiments, the present invention is a method of increasing long bone growth in a human patient, the method comprising the step of administering a CNP conjugate or pharmaceutically acceptable salt thereof at a unit dose from about 6 μg CNP/kg to at least about 150 μg CNP/kg.

In certain embodiments, the present invention is a method of increasing long bone growth in a human patient, the method comprising the step of administering a CNP conjugate or pharmaceutically acceptable salt thereof at a unit dose of 6 μg CNP/kg, 20 μg CNP/kg, 50 μg CNP/kg, 75 μg CNP/kg, 100 μg CNP/kg or 125 μg CNP/kg.

In certain embodiments, the present invention is a method of increasing long bone growth in a human patient, the method comprising the step of administering a CNP conjugate or pharmaceutically acceptable salt thereof at a unit dose of 50 μg CNP/kg to 100 μg CNP/kg.

In certain embodiments, the present invention is a method of increasing long bone growth in a human patient, the method comprising the step of administering a CNP conjugate or pharmaceutically acceptable salt thereof at a unit dose of 50 μg CNP/kg.

In certain embodiments, the present invention is a method of increasing long bone growth in a human patient, the method comprising the step of administering a CNP conjugate or pharmaceutically acceptable salt thereof at a unit dose of 100 μg CNP/kg.

In certain embodiments, the present invention is a method of increasing long bone growth in a human patient, the method comprising the step of administering a CNP conjugate or pharmaceutically acceptable salt thereof of formula (IIf) or (IIf′) at a unit dose of 50 μg CNP/kg to 100 μg CNP/kg.

In certain embodiments, the present invention is a method of increasing long bone growth in a human patient, the method comprising the step of administering a CNP conjugate or pharmaceutically acceptable salt thereof of formula (IIf) or (IIf′) at a unit dose of 50 μg CNP/kg.

In certain embodiments, the present invention is a method of increasing long bone growth in a human patient, the method comprising the step of administering a CNP conjugate or pharmaceutically acceptable salt thereof of formula (IIf) or (IIf′) at a unit dose of 100 μg CNP/kg.

In certain embodiments, administration of the unit dose takes place by subcutaneous injection with a syringe, needle, pen injector or auto-injector. In certain embodiments, administration of the unit dose takes place by subcutaneous injection with a syringe. In certain embodiments, administration of the unit dose takes place by subcutaneous injection with a pen injector. In certain embodiments, administration of the unit dose takes place by subcutaneous injection with an auto-injector.

The present methods can be used for treating or effecting prophylaxis of a patient having or at risk of (e.g., genetic risk) of a disease treatable with CNP. The present methods can also be used for treating a population of patients having or at risk of such a disease. Such a population can include at least 10, 100 or 1000 patients or may represent all patients at a particular institution.

Although clinical trials can be useful for determining doses and dosage regimens de novo, the present methods can also be performed not in the course of a clinical trial. Preferably the regimen used results in a statistically significant improvement (p≤0.05) in at least one sign or symptom of patients (e.g., annualized growth velocity or Δ height SDS) in such a population relative to a contemporary or historical control population receiving placebo. Improvement in a sign or symptom of a disease treatable by CNP can also be monitored in an individual patient compared with baseline measurement(s) before treatment or comparison with mean values from a historical control population. For example, improvement in a sign or symptom of an individual patient can be deemed significant if beyond the mean and one or two standard deviations of the mean value in such a control population in a direction indicating improvement. Although clinical trials can be useful for determining doses and dosage regimens de novo, the present methods can also be performed not in the course of a clinical trial.

Treating refers to curing, reducing or inhibit further deterioration of at least one sign or symptom of a disease or stabilizing at least one sign or symptom of disease. Treating can be determined by comparing sign(s) and symptom(s) in an individual patient before (baseline) and after receiving treatment or by comparing a population of treated patients to a control population as in a clinical trial or trial with an animal model.

Prophylaxis refers to preventing, reducing risk or delaying onset of at least one sign or symptom of disease in a population of patients (or animal models) at risk of the disease administered a drug or CNP conjugate according to the invention compared to a control population of patients (or animal models) at risk of the disease not treated with a drug or CNP conjugate according to the invention. The amount is also considered prophylactically effective if an individual treated patient achieves an outcome more favorable than the mean outcome in a control population of comparable patients not treated by methods of the invention.

In certain embodiments, the present invention is a kit of parts comprising the unit dosage form of the present invention in a vial, a syringe prefilled with a reconstitution solution, a needle for transferring the reconstitution solution from the syringe into the vial, an administration syringe and an injection needle.

The reconstitution solution is a sterile liquid, such as sterile water.

In certain embodiments, a vial adapter could be used instead of the needle for transferring the reconstitution solution from the syringe into the vial and for transferring the reconstituted unit dosage form from the vial to the administration syringe. In certain embodiments, the vial adaptor is a one-piece moulded plastic part with a plastic spike or needle for penetration.

The needle for transferring the reconstitution solution into the vial is a large-bore transfer needle that ensures transfer of the reconstitution solution from the syringe into the vial comprising the unit dosage form of the present invention. The large inner diameter of the needle cannula ensures an increased flow, thereby decreasing the required injection force. In certain embodiments, said needle is a 21 G×25 mm needle.

The injection needle should ensure a comfortable subcutaneous injection, preferably with an injection time of the reconstituted unit dosage form of below 10 s. In other words, the injection needle should have a certain needle gauge and needle length to ensure that the drug is administered in the subcutaneous layer with limited pain. Various injection needles were tested and it was found that the needle length may range from about 3 mm to 13 mm. Also, it was found that injection needles such as a 29 G×8 mm needle, 30 G×12 mm needle or 30 G×4 mm needle meet the requirements above.

In certain embodiments, injection of a highly concentrated reconstituted unit dosage form, such as of a unit dosage form comprising about 60.4 mg/ml CNP conjugate may be challenging due to the high viscosity of the CNP conjugate. Applicant surprisingly found that by using a 30 G×12 mm or 30 G×4 mm needle even highly concentrated unit dosage forms can be administered.

In certain embodiments, the present invention is a kit of parts comprising a pen injector or auto-injector and a vial comprising the unit dosage of the present invention, said vial being in the form of a pre-filled cartridge for use in the pen injector or auto-injector.

In certain embodiments, the present invention is a pen injector or auto-injector comprising the unit dosage form of the present invention.

The present invention also provides for a CNP, CNP conjugate or pharmaceutically acceptable salt thereof (such as a CNP or CNP conjugate or pharmaceutically acceptable salt thereof as described herein), or unit dosage form (as described herein), which is administered to a patient to provide a sustained exposure of free plasma CNP (free CNP) at an efficacious level between administrations for use in the therapeutic methods disclosed herein. Suitably, said CNP, CNP conjugate or pharmaceutically acceptable salt thereof or unit dosage form is administered via a dosing frequency which sustains plasma free CNP concentration at a therapeutic level between successive doses, such as a plasma free CNP concentration of at least about 1 pmol/L. It will be understood that a sustained-release may be obtained by treatment with repeat dosing of a therapeutically effective amount of a CNP conjugate or pharmaceutically acceptable salt thereof.

Suitably, via repeated administrations of the CNP or CNP conjugate or pharmaceutically acceptable salt thereof or unit dosage form, e.g. daily, at least daily, weekly, at least weekly, monthly or at least monthly, the sustained exposure of free CNP is for a period of at least 6 months, such as at least 9 months, such as at least 1 year.

It is recognized that during the initial treatment period, for example the first 1, 2 or 3 months from initiation of treatment (in treatment naïve patients), the minimum free CNP concentration in plasma may gradually increase after each successive dose (which may be referred to as a run in period), until a sustained therapeutically effective exposure level is maintained, that is, trough and peak values of CNP are at a steady state as a result of administration and elimination amounts being about equal through repeated cycles of administration.

A sustained exposure may be obtained when the minimum free CNP concentration in plasma between successive administrations, also referred to as trough concentration, remains at a level which is at least about 1 pmol/L, or at least about 1.5 pmol/L, or about at least 5 pmol/L, or about 8 pmol/L, or about 15 pmol/L. Alternatively, a sustained exposure may be obtained when the minimum free CNP concentration in plasma between successive administrations, also referred to as trough concentration, remains at a level which is at least about 1.4 pmol/L, or at least about 1.6 pmol/L, or about at least 6.5 pmol/L, or about 9.4 pmol/L, or about 19.7 pmol/L.

As illustrated by the data from the clinical trial reported herein, such levels of sustained free CNP-38 exposure were found to be therapeutically effective.

In certain embodiments, a sustained exposure may be obtained when the minimum free CNP concentration in plasma between successive administrations, also referred to as trough concentration, such as at least about 1.4 pmol/L, or at least about 1.6 pmol/L, or about at least 6.5 pmol/L, or about 9.4 pmol/L, or about 19.7 pmol/L.

In certain embodiments, the minimum free CNP concentration in plasma between successive administrations, also referred to as trough concentration, remains at a level of at least about 1.8 pmol/L, or at least about 2.3 pmol/L, or about at least 8.4 pmol/L, or about 12 pmol/L, or about pmol/l. In certain embodiments, the minimum free CNP concentration in plasma between successive administrations, also referred to as trough concentration, remains at a level of at least about 2.7 pmol/L, or at least about 2.4 pmol/L, or about at least 9.7 pmol/L, or about 14.1 pmol/L, or about 29 pmol/L. In certain embodiments, the minimum free CNP concentration in plasma between successive administrations, also referred to as trough concentration, remains at a level of at least about 2.7 pmol/L, or at least about 2.4 pmol/L, or about at least 9.7 pmol/L, or about 14.1 pmol/L, or about 29 pmol/L.

In certain embodiments, the CNP, CNP conjugate or pharmaceutically acceptable salt thereof or unit dosage form is administered in a regimen (route of administration, frequency and amount) to achieve plasma levels of free CNP in which at steady state, troughs range from 1.8 pmol/L to about 29 pmol/L and peaks range from about 30 pmol/L to about 100 pmol/L. In some embodiments, troughs range from about 8 pmol/L to about 29 pmol/L and peaks range from about 30 pmol/L to about 50 pmol/L. In some embodiments, troughs range from about 18 pmol/L to about 29 pmol/L and peaks range from about 30 pmol/L to about 50 pmol/L. In certain embodiments, the ratio of peaks to troughs is no more than 1.5:1, 2:1 or 3:1. Optionally administration is weekly and by a subcutaneous route. In certain embodiments, free CNP concentrations are measured in a patient. Levels can be measured at baseline before treatment and one or more times after treatment commences.

The present invention also provides for a method for reducing the frequency of achondroplasia related adverse events in a patient diagnosed with achondroplasia, said method comprising administering a therapeutically effective amount of a CNP, CNP conjugate or pharmaceutically acceptable salt thereof or unit dosage form, wherein optionally the patient may be a pediatric patient, and/or a patient with open bone epiphysis.

In certain embodiments, the achondroplasia-related adverse event is selected from the group consisting of sleep apnea syndrome, ear infection, foramen magnum stenosis and kyphosis. In certain embodiments, the achondroplasia-related adverse event is selected from the group consisting of sleep apnea syndrome, ear infection, foramen magnum stenosis and kyphosis.

The present invention also provides for a method for treating sleep apnea syndrome in a patient in need to said treatment, said method comprising administering a therapeutically effective amount of a CNP, CNP conjugate or pharmaceutically acceptable salt thereof or unit dosage form to the patient. By way of example, the treatment may reduce the incidence of sleep apnea or reduce the severity of sleep apnea.

The present invention also provides for a method for treating ear infection in a patient or for reducing incidence of ear infection in a patient, in a patient in need of said treatment, said method comprising administering a therapeutically effective amount of a CNP, CNP conjugate or pharmaceutically acceptable salt thereof or unit dosage form to the patient thereby treating ear infection or reducing incidence of ear infection.

The present invention also provides for a method for treating foramen magnum stenosis in a patient in need of said treatment, said method comprising administering an effective amount of CNP, CNP conjugate or pharmaceutically acceptable salt thereof or unit dosage form to the patient thereby treating foramen magnum stenosis.

The present invention also provides for a method for treating kyphosis in a patient in need of said treatment, said method comprising administering an effective amount of CNP, CNP conjugate or pharmaceutically acceptable salt thereof or unit dosage form to the patient thereby treating kyphosis. In certain embodiments, the patient has been diagnosed with a bone dysplasia or bone disorder, such as a disorder selected from the group consisting of achondroplasia, hypochondroplasia, short stature, Noonan syndrome and SHOX deficiency.

In certain embodiments, the patient has been diagnosed with achondroplasia. In certain embodiments, the patient is a pediatric patient with open bone epiphysis.

In certain embodiments, the patient is aged up to 18 years of age or is less than 18 years of age. In certain embodiments, the patient is aged up to 16 years of age or is less than 16 years of age. In certain embodiments, the patient is aged up to 14 years of age or is less than 14 years of age. In certain embodiments, the patient is aged up to 10 years of age or is less than 10 years of age. In certain embodiments, the patient is aged up to 5 years of age or is less than 5 years of age. In certain embodiments, the patient is an achondroplasia patient aged up to 2-5 years of age, such as 2, 3 or 4 years of age. In certain embodiments, the patient is an achondroplasia patient at least 6 months of age, such as at least 1 year of age or at least 2 years of age. In certain embodiments, the patient is an achondroplasia patient with open bone epiphysis. In certain embodiments, the patient is an achondroplasia patient aged up to 18 years of age or is less than 18 years of age. In certain embodiments, the patient is an achondroplasia patient aged up to 16 years of age or is less than 16 years of age. In certain embodiments, the patient is an achondroplasia patient aged up to 14 years of age or is less than 14 years of age. In certain embodiments, the patient is an achondroplasia patient aged up to 10 years of age or is less than years of age. In certain embodiments, the patient is an achondroplasia patient aged up to 5 years of age or is less than 5 years of age. In certain embodiments, the patient is an achondroplasia patient up to 2-5 years of age, such as 2, 3 or 4 years of age. In certain embodiments, the patient is an achondroplasia patient at least 6 months of age, such as at least 1 year of age or at least 2 years of age.

The invention is further described by the following non-limiting items.
1. A unit dosage form comprising a therapeutically effective amount of a CNP conjugate or pharmaceutically acceptable salt thereof in which a CNP moiety is reversibly conjugated to a polymeric moiety.
2. The unit dosage form of item 1, wherein the unit dosage form comprises a unit dose.
3. The unit dosage form of item 2, wherein the unit dose ranges from 50 μg to 7000 μg of CNP.
4. The unit dosage of item 2 or 3, wherein the unit dose ranges from 100 μg to 5000 μg of CNP.
5. The unit dosage of any one of items 2 to 4, wherein the unit dose ranges from about 6 μg CNP/kg to at least about 100 μg CNP/kg.
6. The unit dosage of any one of items 1 to 5, wherein a patient's body weight ranges from about 2 kg to about 80 kg.
7. The unit dosage of any one of items 1 to 6, wherein a patient's body weight ranges from about 4 kg to about 60 kg.
8. The unit dosage form of any one of items 2 to 7, wherein the unit dose is about 6 μg CNP/kg.
9. The unit dosage form of any one of items 2 to 7, wherein the unit dose is about 20 μg CNP/kg.
10. The unit dosage form of any one of items 2 to 7, wherein the unit dose is about 50 μg CNP/kg.
11. The unit dosage form of any one of items 2 to 7, wherein the unit dose is about 100 μg CNP/kg.
12. The unit dosage form of any one of items 2 to 7, wherein the unit dose is about 125 μg CNP/kg.
13. The unit dosage form of any one of items 2 to 12, wherein the unit dosage form is dry or liquid.
14. The unit dosage form of any one of items 1 to 13, wherein the unit dosage form is liquid.
15. The unit dosage form of any one of items 1 to 14, wherein the pH of the unit dosage form ranges from about 4 to 6.
16. The unit dosage form of any one of items 1 to 15, wherein the pH of the unit dosage form is about 5.
17. The unit dosage form of any one of items 2 to 16, wherein the volume of the unit dose ranges from about 0.01 ml to about 1.1 ml.
18. The unit dosage form of any one of items 2 to 17, wherein the volume of the unit dose ranges from about 0.01 ml to about 0.75 ml.
19. The unit dosage form of any one of items 2 to 18, wherein the volume of the unit dose ranges from about 0.01 ml to about 0.50 ml.
20. The unit dosage form of any one of items 1 to 19, wherein the unit dosage form is contained within a single vial.
21. The unit dosage form of any one of items 2 to 20, wherein upon administration of the unit dose the incidence of hypotension is less than 10%, preferably less than 8%, most preferably less than 5%, even more preferably is less than 3%.
22. The unit dosage form of any one of claims 1 to 21, wherein the CNP conjugate or pharmaceutically acceptable salt thereof is a compound of formula (Ia) or (Ib):

Z [symbol] L²-L¹-D)ₓ (Ia),

D [symbol] L¹-L²-Z)_{y} (Ib),
wherein
   -D is a CNP moiety;
   -L¹- is a reversible linker moiety;
   -L²- is a single chemical bond or a spacer moiety;
   —Z is a polymeric moiety;
   x is an integer selected from the group consisting of 1, 2, 3, 4, 5, 6, 7, 8, 9, 10, 11, 12, 13, 14, 15 and 16; and
   y is an integer selected from the group consisting of 1, 2, 3, 4 and 5.
23. The unit dosage form of any one of items 1 to 22, wherein the CNP conjugate or pharmaceutically acceptable salt is a compound of formula (IIf):

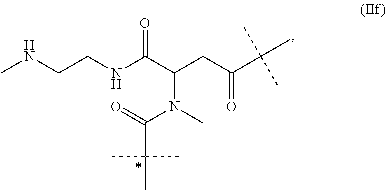

wherein
   the unmarked dashed line indicates the attachment to a nitrogen provided by the side chain of the lysine at position 26 of a CNP moiety of SEQ ID NO:24 by forming an amide bond; and
   the dashed line marked with the asterisk indicates attachment to —Z having the structure

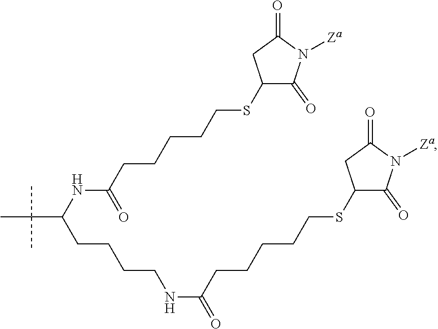

wherein
each —Z^{a} is

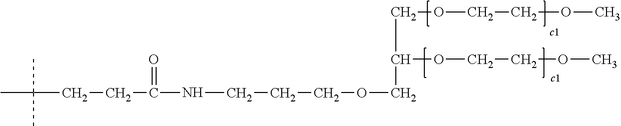

wherein
each c1 is an integer independently ranging from 200 to 250.
24. The unit dosage form of any one of items 1 to 23, wherein the CNP conjugate is a compound of formula (IIf′):

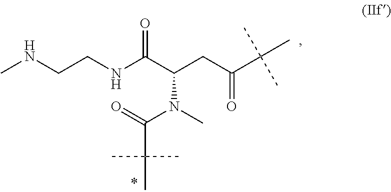

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

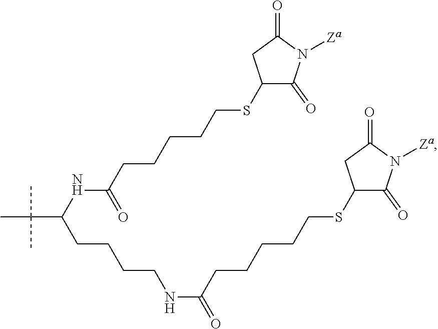

wherein
each Z^{a} is

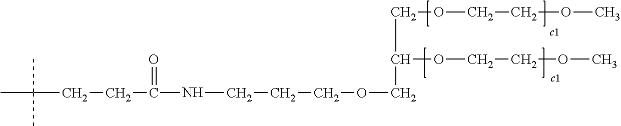

wherein
each c1 is an integer independently ranging from 200 to 250.
25. The unit dosage form of any one of items 1 to 24, wherein the CNP conjugate is a compound of formula (IIf′):

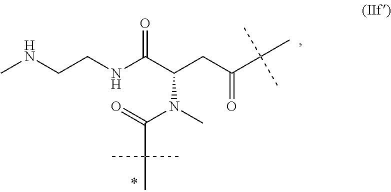

wherein
the unmarked dashed line indicates the attachment to a nitrogen provided by the side chain of the lysine at position 26 of the CNP moiety of SEQ ID NO:24 by forming an amide bond; and
the dashed line marked with the asterisk indicates attachment to —Z having the structure

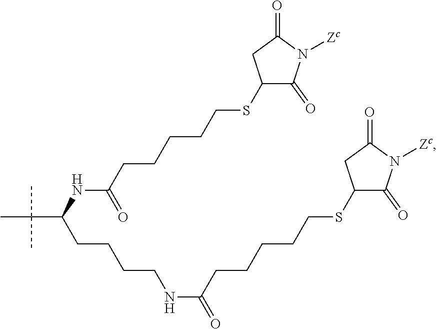

wherein
each Z^{a} is

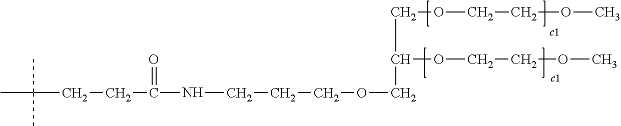

wherein
each c1 is an integer independently ranging from 200 to 250.
26. The unit dosage form of any one of items 1 to 25, wherein each c1 is an integer independently ranging from 220 to 245.
27. The unit dosage form of any one of items 1 to 26, wherein each c1 is about 225.
28. The unit dosage form of any one of items 1 to 27, for use in the treatment of one or more diseases which can be treated by CNP.
29. The unit dosage form of any one of items 1 to 28, for use in the treatment of achondroplasia.
30. A method of treating, reducing the risk of or delaying in a human patient a disease treatable by CNP, the method comprising the step of administering the unit dosage form of any one of claims 1 to 27.
31. The method of item 30, wherein the step of administering the unit dosage does not occur in the course of a clinical trial.
32. The method of item 30, wherein the unit dosage form is administered subcutaneously.
33. The method of item 30 or 32, wherein the unit dosage form is administered weekly.
34. The method of item 30 or 32, wherein the unit dosage form is administered weekly for at least a year.
35. The method of item 30, 33 or 34, wherein the unit dosage form is administered weekly until the patient is 18 years old.
36. The method of any one of items 30 or 32 to 35, wherein the disease is selected from the group consisting of achondroplasia, hypochondroplasia, short stature, Noonan syndrome and SHOX deficiency.
37. The method of any one of items 30 or 32 to 35, wherein the disease is achondroplasia.
38. The method of any one of items 30 or 32 to 37 for treating a population of human patients, wherein each patient in the population is administered the unit dosage form of any one of claims 1 to 27.

The invention is also further described by the following non-limiting items.
1. A method of treating achondroplasia in a human patient, the method comprising the step of administering a CNP conjugate or pharmaceutically acceptable salt thereof at a unit dose of 100 μg CNP/kg, wherein the CNP conjugate is a compound of formula (IIf′):

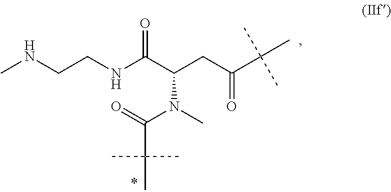

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

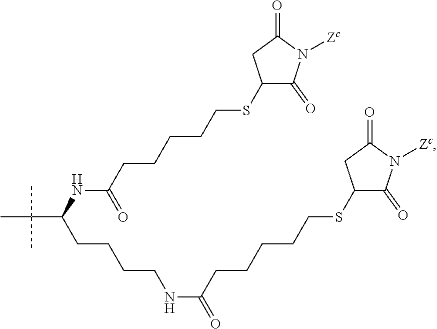

wherein
each Z^{c} is

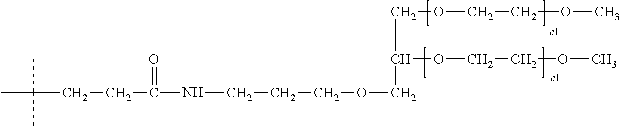

and wherein
each c1 is an integer independently ranging from 200 to 250,
wherein the unit dose is administered weekly.
2. A method of increasing growth velocity in a human patient, the method comprising the step of administering a CNP conjugate or pharmaceutically acceptable salt thereof of (a) formula (IIf):

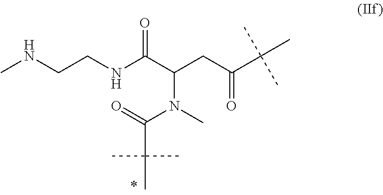

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

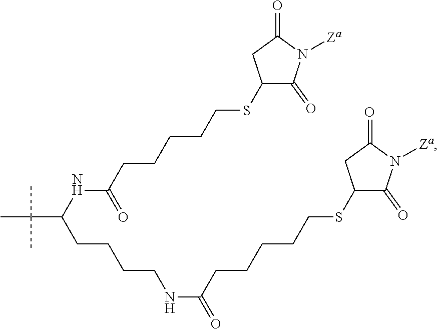

wherein
each Z^{a} is

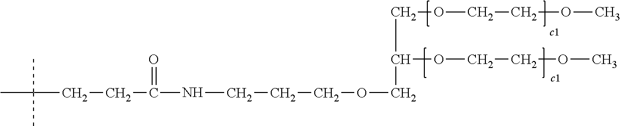

and wherein
each c1 is an integer independently ranging from 200 to 250,
or
(b) formula (IIf):

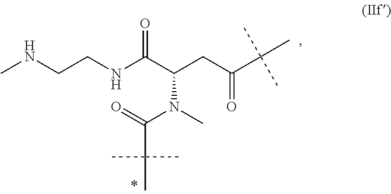

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

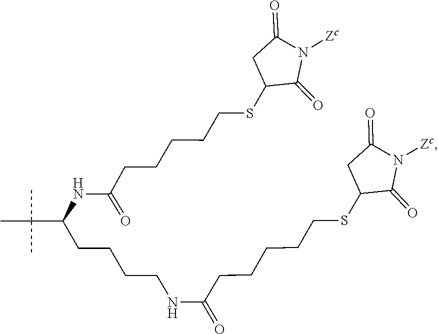

wherein
each Z^{c} is

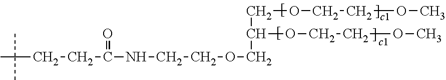

and wherein
each c1 is an integer independently ranging from 200 to 250,
at a unit dose of 100 μg CNP/kg, wherein the unit dose is administered weekly.
3. A method of increasing long bone growth in a human patient, the method comprising the step of administering a CNP conjugate or pharmaceutically acceptable salt thereof of formula (IIf) or (IIf′), wherein each of (IIf) and (IIf′) are as defined in item 2, at a unit dose of 100 μg CNP/kg, wherein the unit dose is administered weekly.
4. A method of treating a skeletal dysplasia, such as achondroplasia, said method comprising administering a unit dosage form comprising a CNP conjugate or pharmaceutically acceptable salt thereof of formula (IIf) or (IIf′), wherein each of (IIf) and (IIf′) are as defined in item 2, at a unit dose of 100 μg CNP/kg, wherein said unit dosage form is administered weekly to a human patient with open epiphysis and said treatment reduces the incidence of achondroplasia-related adverse events in the human patient.
5. A method of increasing growth velocity in an achondroplasia human patient, the method comprising the step of administering a CNP conjugate or pharmaceutically acceptable salt thereof of formula (IIf) or (IIf′), wherein each of (IIf) and (IIf′) are as defined in item 2, at a unit dose of 100 μg CNP/kg for a period of at least 52 weeks, wherein said unit dosage form is administered to a human patient with open epiphysis and said treatment reduces the incidence of achondroplasia-related adverse events in the human patient
6. A CNP conjugate or pharmaceutically acceptable salt thereof for use in a method of treating achondroplasia in a human patient, the method comprising the step of administering the CNP conjugate or pharmaceutically acceptable salt thereof at a unit dose of 100 μg CNP/kg, wherein the CNP conjugate is a compound of formula (IIf′):

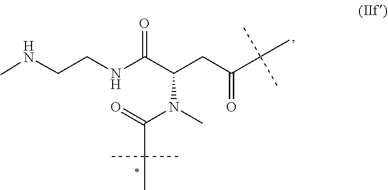

wherein
the unmarked dashed line indicates the attachment to a nitrogen provided by the side chain of the lysine at position 26 of the CNP moiety of SEQ ID NO:24 by forming an amide bond; the dashed line marked with the asterisk indicates attachment to —Z having the structure

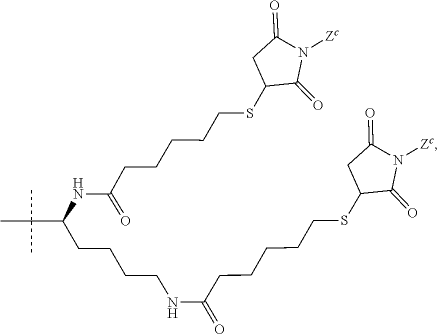

wherein
each Z^{c} is

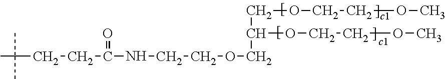

and wherein
each c1 is an integer independently ranging from 200 to 250,
wherein the unit dose is administered weekly.
7. A CNP conjugate or pharmaceutically acceptable salt thereof for use in a method of increasing growth velocity in a human patient, the method comprising the step of administering the CNP conjugate or pharmaceutically acceptable salt thereof of (a) formula (IIf):

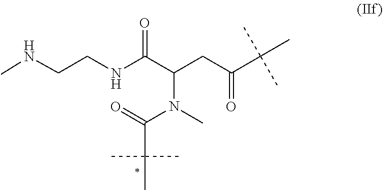

wherein
the unmarked dashed line indicates the attachment to a nitrogen provided by the side chain of the lysine at position 26 of the CNP moiety of SEQ ID NO:24 by forming an amide bond; the dashed line marked with the asterisk indicates attachment to —Z having the structure

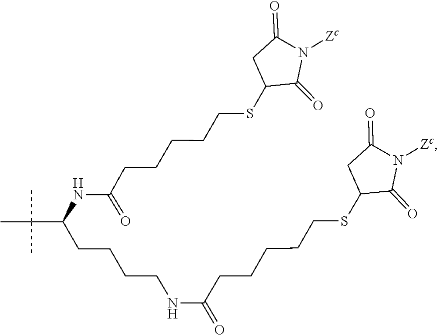

wherein
each Z^{a} is

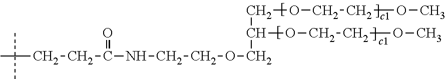

and wherein
each c1 is an integer independently ranging from 200 to 250
or
(b) formula (IIf′):

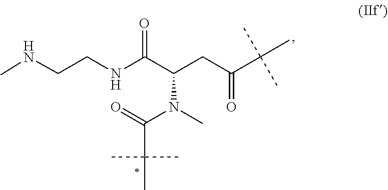

wherein
the unmarked dashed line indicates the attachment to a nitrogen provided by the side chain of the lysine at position 26 of the CNP moiety of SEQ ID NO:24 by forming an amide bond;
the dashed line marked with the asterisk indicates attachment to —Z having the structure

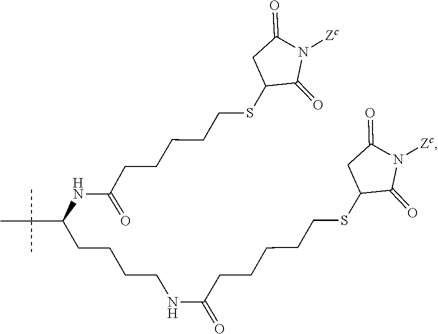

wherein
each —Z^{c} is

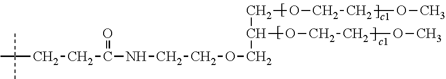

and wherein
each c1 is an integer independently ranging from 200 to 250
at a unit dose of 100 μg CNP/kg, wherein the unit dose is administered weekly.
8. A CNP conjugate or pharmaceutically acceptable salt thereof for use in a method of increasing long bone growth in a human patient, the method comprising the step of administering a CNP conjugate or pharmaceutically acceptable salt thereof of formula (IIf) or (IIf′) wherein each of (IIf) and (IIf′) are as defined in item 7, at a unit dose of 100 μg CNP/kg, wherein the unit dose is administered weekly.
9. A CNP conjugate or pharmaceutically acceptable salt thereof for use in a method of treating a skeletal dysplasia, such as achondroplasia, said method comprising administering a unit dosage form comprising a CNP conjugate or pharmaceutically acceptable salt thereof of formula (IIf) or (IIf′), wherein each of (IIf) and (IIf′) are as defined in item 7, at a unit dose of 100 μg CNP/kg, wherein said unit dosage form is administered weekly to a human patient with open epiphysis and said treatment reduces the incidence of achondroplasia-related adverse events in the human patient.
10. A CNP conjugate or pharmaceutically acceptable salt thereof for use in a method of increasing growth velocity in a achondroplasia human patient, the method comprising the step of administering a CNP conjugate or pharmaceutically acceptable salt thereof of formula (IIf) or (IIf′), wherein each of (IIf) and (IIf′) are as defined in item 7, at a unit dose of 100 μg CNP/kg for a period of at least 52 weeks, wherein said unit dosage form is administered to a human patient with open epiphysis and said treatment reduces the incidence of achondroplasia-related adverse events in the human patient.
11. The method of any one of items 2 to 5, or the CNP conjugate or pharmaceutically acceptable salt thereof for use of any one of items 7 to 10, wherein the CNP conjugate or pharmaceutically acceptable salt thereof is of formula (IIf′):

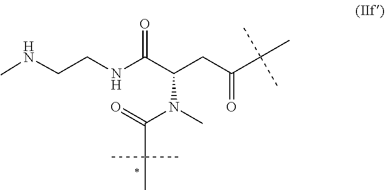

wherein
the unmarked dashed line indicates the attachment to a nitrogen provided by the side chain of the lysine at position 26 of the CNP moiety of SEQ ID NO:24 by forming an amide bond; the dashed line marked with the asterisk indicates attachment to —Z having the structure

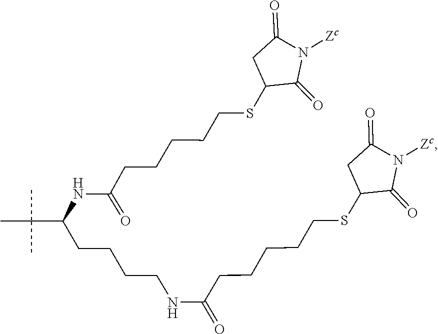

wherein
each Z^{c} is

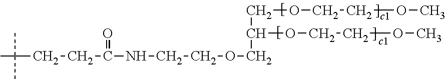

and wherein
each c1 is an integer independently ranging from 200 to 250.
12. The method of any one of items 2 to 4, or the CNP conjugate or pharmaceutically acceptable salt thereof for use of any one of items 7 to 9, wherein the patient has achondroplasia.
13. The method of any one of items 1 to 5, 11 or 12, or the CNP conjugate or pharmaceutically acceptable salt thereof for use of any one of items 6 to 10, 11 or 12, wherein the patient is a pediatric patient or an infant.
14. The method of item 13 or the CNP conjugate or pharmaceutically acceptable salt thereof for use of item 13, wherein the pediatric patient is aged 2 to 10 years, such as 2 to 5 years or such as 5 to 10 years.
15. The method of any one of items 1 to 5 or 11 to 14, or the CNP conjugate or pharmaceutically acceptable salt thereof for use of any one of embodiments 6 to 14, wherein the CNP conjugate or pharmaceutically acceptable salt thereof is administered subcutaneously.
16. The method of any one of items 1 to 5 or 11 to 15, or the CNP conjugate or pharmaceutically acceptable salt thereof for use of any one of embodiments 6 to 15, wherein the unit dosage form comprises succinic acid and trehalose.

### EXAMPLES

### Materials and Methods

All materials were commercially available except where stated otherwise.

### Example 1: Synthesis of Compound (1)

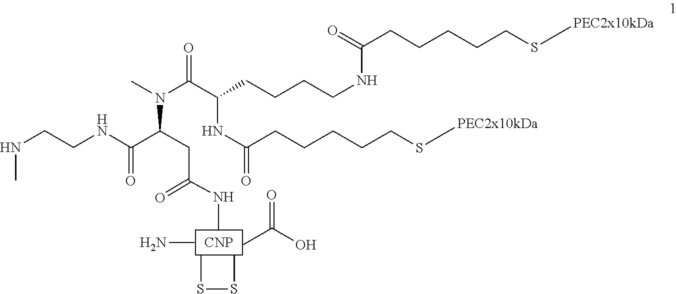

Compound (1) was synthesized as described in WO2017/118693 for conjugate 11i.

Compound (1) can also be represented as follows:

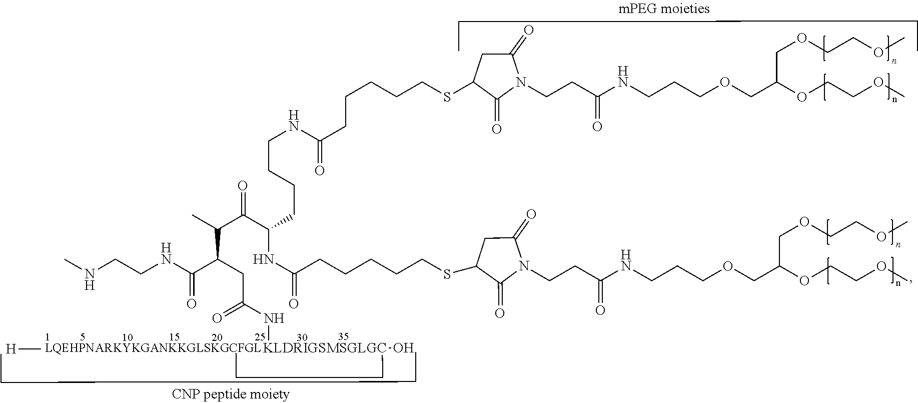

wherein n ranges from about 200 to about 250, such as from 200 to 250 corresponding to about kDa.

Compound (1) is a long-acting essentially inactive prodrug consisting of CNP-38 transiently bound to a carrier molecule, mPEG via a reversible linker. mPEG acts as an inert carrier, extending CNP-38 circulation time in the body through a shielding effect that minimizes CNP-38 clearance and largely inactivates CNP-38 until its release. Compound (1) releases active CNP via auto-cleavage of the reversible linker in a controlled-manner based on physiologic pH and temperature. As such, compound (1) is designed to provide sustained exposure of active CNP over 7 days, allowing an optimal pharmacokinetic profile for use in ACH. The drug product formulation comprising compound (1) is a lyophilized powder in a single-use vial, i.e. compound (1) 3.9 mg CNP-38/vial. Prior to use, the lyophilized powder must be reconstituted with sterile water for injection from a prefilled syringe. After reconstitution the concentration is 3.6 mg CNP-38/ml. This solution will be administered by subcutaneous injection via syringe and needle. With reference to compound (1), the dose level CNP/kg or CNP-38/kg refers to the amount of CNP moiety (pg) present in the dose of compound (1), per kilogram of patient's bodyweight.

### Example 2—Phase 1 Study

To date, a Phase 1, randomized, double-blind, placebo-controlled, single-ascending dose trial in healthy adult male subjects has been completed. This trial was designed to evaluate the safety, tolerability, and pharmacokinetics of single subcutaneous unit doses of compound (1) up to 150 μg CNP/kg. Compound (1) was generally well tolerated at all investigated doses. A single dose of compound (1) delivered free CNP-38 in a dose-dependent manner with a long and dose-independent mean apparent t_{1/2} of 120 h. The most commonly reported (>10% of subjects) treatment-emergent adverse events (TEAEs) among subjects who received compound (1) were headache, contact dermatitis, postural dizziness, (orthostatic) tachycardia, dizziness, and orthostatic hypotension. Most TEAEs were mild or moderate. There were no serious adverse effects (AEs) or deaths reported and none of the AEs were considered to be dose-limiting or led to trial discontinuation. Local tolerability findings were predominantly mild to moderate redness. Two AEs related to injection sites, mild injection site pain (10 μg CNP/kg) and mild injection site discomfort (75 gg CNP/kg) were reported. No clinically relevant dose-dependent trends were observed on blood pressure, heart rate, or safety laboratory test results at all investigated doses. Also, no evidence of immunogenic response was observed in any subject.

### Example 3—Phase 2 Study

A Phase 2, multicenter, double-blind, randomized, placebo-controlled, dose escalation trial evaluating safety, efficacy, and pharmacokinetics of subcutaneous doses of compound (1) administered once weekly for 52 weeks in prepubertal children with achondroplasia followed by an Open-Label Extension Period. The study enrolled a minimum of 60 male and female prepubertal children with ACH aged 2 to 10 years old. The clinical trial is listed on www.clinicaltrials.gov under the ClinicalTrials.gov Identifier: NCT04085523. The objectives of the study were as follow:

### Primary:

In prepubertal children with achondroplasia (ACH) at 52 weeks
To determine the safety of once weekly subcutaneous (SC) doses of compound (1)
To evaluate the effect of once weekly SC doses of compound (1) on annualized height velocity (AHV)

### Secondary:

To evaluate the effect of once weekly SC doses of compound (1) on body proportionality (upper to lower body segment ratio) in prepubertal children with ACH at 52 weeks
To evaluate the pharmacokinetic (PK) properties of once weekly SC doses of compound (1)
To assess the potential immunogenic response to once weekly SC doses of compound (1)

### Inclusion Criteria

1. Clinical diagnosis of ACH with genetic confirmation
2. Age between 2 to 10 years old (inclusive) at Screening Visit
3. Prepubertal (Stage 1 breasts for girls or testicular volume <4 ml for boys) at Screening Visit
4. Able to stand without assistance
5. Caregiver willing and able to administer subcutaneous injections of study drug
6. Written, signed informed consent of the parent(s) or legal guardian(s) of the participant and written assent of the participant as required by the institutional review board/human research ethics committee/independent ethics committee (IRB/HREC/IEC)

### Exclusion Criteria

1. Clinically significant findings at Screening that:
   are expected to require surgical intervention during participation in the trial or
   are musculoskeletal in nature, such as Salter-Harris fractures and severe hip pain or
   otherwise are considered by investigator or Medical Monitor to make a participant unfit to receive study drug or undergo trial related procedures
2. Have received treatment (>3 months) of human growth hormone (hGH) or other medications known to affect stature or body proportionality at any time
3. Have received any dose of medications intended to affect stature or body proportionality within the previous 6 months of Screening Visit
4. Have received any study drug or device intended to affect stature or body proportionality at any time
5. History or presence of injury or disease of the growth plate(s), other than ACH, that affects growth potential of long bones
6. History of any bone-related surgery that affects growth potential of long bones, such as orthopedic reconstructive surgery and osteotomy (Limb-lengthening with full recovery is allowed with a minimum of 12 months of bone healing. Foramen magnum decompression and laminectomy with full recovery are allowed with minimum of 6 months of bone healing. History of 8 plate epiphysiodesis is allowed, but the plates must have been removed prior to Screening with minimum 4 weeks of healing.)
7. Have a form of skeletal dysplasia other than ACH or known medical conditions that result in short stature or abnormal growth [such as severe achondroplasia with developmental delay and acanthosis nigricans (SADDAN), hypochondroplasia, growth hormone deficiency, Turner syndrome, pseudoachondroplasia, inflammatory bowel disease, chronic renal insufficiency, active celiac disease¹, Vitamin D deficiency², untreated hypothyroidism³, poorly controlled diabetes mellitus (HbA1c≥8.0%) or diabetic complications⁴]¹Celiac disease responsive to a gluten-free diet is allowed²Vitamin D deficiency or insufficiency treated with supplementation is allowed. Vitamin D deficiency is defined as 25(OH)D level <20 ng/mL (<49.9 nmol/L), insufficiency is defined as 25(OH)D level 20-30 ng/mL (49.92-74.86 nmol/L). Participants with Vitamin D deficiency or insufficiency must be on Vitamin D regimen before randomization.³Participants with hypothyroidism must be clinically euthyroid for 3 months prior to Screening Visit and, in the opinion of the investigator, have achieved any catch-up growth expected from thyroxine replacement⁴Participants with diabetes mellitus must have been on stable medication regimen for 3 months prior to randomization (dose adjustments are allowed but addition or discontinuation of medications in this time period is disallowed)
8. History or presence of malignant disease, other than basal cell epithelioma/carcinoma or completely resected squamous skin cancer with no recurrence for 12 months per medical records
9. History or presence of the following:
   Chronic anemia (resolved iron deficiency anemia is allowed)
   Significant cardiovascular disease per the judgement of the investigator, such as congenital heart disease (uncomplicated patent ductus arteriosus and atrial or ventricular septal defect with repair are allowed), aortic insufficiency, clinically significant arrhythmias, congestive heart failure with NYHA class II and above, or other conditions that impair regulation of blood pressure or heart rate
   Condition that impacts hemodynamic stability (such as autonomic dysfunction, orthostatic intolerance)
   History of chronic renal insufficiency
   Chronic or recurrent illness that can affect hydration or volume status. This may include conditions associated with decreased nutritional intake or increased volume loss
   Bone fracture within 6 months prior to Screening Visit (within 2 months for fracture of digits)
   Any disease or condition that, in the opinion of the investigator, may make the participant unlikely to fully complete the trial, may confound interpretation of trial results, or presents undue risk from receiving study drug
10. Child has significant electrocardiogram abnormalities, including evidence of a previous myocardial infarction, left ventricular hypertrophy, flat T waves (particularly in the inferior leads) or more than minor non-specific ST-T wave changes or:
   QRS>90 milliseconds (msec)
   QT interval corrected using Fridericia's formula (QTcF)>440 msec
   PR interval >170 msec
   Complete right or left bundle branch block
11. Requires, or anticipated to require, chronic (>4 weeks) or repeated (more than twice per year) treatment with oral corticosteroids during participation in the trial (low and mid-dose inhaled corticosteroids are allowed. High-dose inhaled corticosteroids are not allowed).
12. Use of medication known to prolong the QT/QTc interval within 7 days or 5 half-lives (whichever is longer) prior to the Screening Visit.
13. Ongoing treatment with any medication that affects blood pressure or heart rate
14. Known hypersensitivity to the components of the study drug [trehalose, tris(hydroxymethyl)aminomethane, succinate and PEG]
15. Any other reason that in the opinion of the investigator would prevent the child from complying with the trial requirements or completing the trial

This trial is a multicenter trial consisting of two treatment periods. A 52 week double-blind, randomized, placebo controlled, dose escalation trial evaluating up to 5 different dose levels of weekly compound (1) administered subcutaneously in prepubertal children 2 to 10 years old, inclusive, with ACH.

TABLE 1
Unit doses of CNP

| | | Approximate Number |
|---|---|---|
| Cohort | Planned Dose Level | Of Participants |
| 1 | 6 μg CNP/kg/week | 9 on compound (1), 3 on |
| | | placebo |
| 2 | 20 μg CNP/kg/week | ≥9 on compound (1), ≥3 on |
| | | placebo |
| 3 | 50 μg CNP/kg/week | ≥9 on compound (1), ≥3 on |
| | | placebo |
| 4 | 100 μg CNP/kg/week | ≥9 on compound (1), ≥3 on |
| | | placebo |
| 5 | >100 μg | ≥9 on compound (1), ≥3 on |
| | CNP/kg/week* | placebo |

*At a dose to be determined, if needed, based on emerging data

TABLE 2
Baseline demographics-status end of 2021

| | Cohort 1 | Cohort 2 | Cohort 3 |
|---|---|---|---|
| Demographics | (N = 13) | (N = 15) | (N = 14) |
| Age (years) | | | |
| Mean (SD) | 6.4 (2.82) | 6.2 (3.13) | 5.6 (2.91) |
| Age Group | | | |
| (years), n (%) | | | |
| 2-5 years | 5 (38.5) | 7 (46.7) | 6 (42.9) |
| 5-8 years | 4 (30.8) | 5 (33.3) | 4 (28.6) |
| >8 years | 4 (30.8) | 3 (20.0) | 4 (28.6) |
| Sex, n (%) | | | |
| Female | 7 (53.8) | 5 (33.3) | 5 (35.7) |
| Male | 6 (46.2) | 10 (66.7) | 9 (64.3) |
| Age at ACH | | | |
| Diagnosis, n (%) | | | |
| Pre-birth | 0 | 1 (6.7) | 4 (28.6) |
| At Birth | 4 (30.8) | 3 (20.0) | 2 (14.3) |
| 0-6 months | 9 (69.2) | 9 (60.0) | 7 (50.0) |
| >6-12 months | 0 | 2 (13.3) | 1 (7.1) |
| >12 months | 0 | 0 | 0 |
| Height SDS | | | |
| Mean SD | −5.4 (1.10) | −5.0 (0.66) | −4.7 (0.82) |

Table 2 describes the baseline demographics for cohorts 1, 2 and 3, with each cohort also containing placebo subjects. Following completion of the trial and unblinding of the data, the baseline demographics for cohorts 1, 2, 3 and 4 and the placebo subjects is displayed in Table 3.

TABLE 3
Baseline demographics following unblinding of the trial

| | | | | | Pooled |
|---|---|---|---|---|---|
| | Cohort 1 | Cohort 2 | Cohort 3 | Cohort 4 | Placebo |
| Demographics | (N = 10) | (N = 11) | (N = 10) | (N = 11) | (N = 15) |
| Age (years) | | | | | |
| Mean (SD) | 6.5 (2.59) | 6.3 (2.90) | 5.2 (2.99) | 5.8 (2.61) | 5.9 (3.11) |
| Age Group | | | | | |
| (years), n (%) | | | | | |
| 2-5 years | 3 (30.0) | 5 (45.5) | 5 (50.0) | 3 (27.3) | 8 (53.3) |
| 5-8 years | 4 (40.0) | 4 (36.4) | 3 (30.0) | 6 (54.5) | 2 (13.3) |
| >8 years | 3 (30.0) | 2 (18.2) | 2 (20.0) | 2 (18.2) | 5 (33.3) |
| Sex, n (%) | | | | | |
| Female | 7 (70.0) | 3 (27.3) | 3 (30.0) | 6 (54.5) | 5 (33.3) |
| Male | 3 (30.0) | 8 (72.7) | 7 (70.0) | 5 (45.5) | 10 (66.7) |
| Age at ACH | | | | | |
| Diagnosis, | | | | | |
| n (%) | | | | | |
| Pre-birth | 0 | 1 (9.1) | 2 (20.0) | 0 | 3 (20.0) |
| At Birth | 2 (20.0) | 4 (36.4) | 2 (20.0) | 1 (9.1) | 5 (33.3) |
| 0-6 months | 8 (80.0) | 4 (36.4) | 6 (50.0) | 8 (72.7) | 7 (46.7) |
| >6-12 months | 0 | 2 (18.2) | 0 | 1 (9.1) | 0 |
| >12 months | 0 | 0 | 0 | 1 (9.1) | 0 |
| Height SDS | | | | | |
| Mean SD | −5.5 | −4.9 | −4.8 | −4.9 | −4.9 |
| | (1.05) | (0.67) | (0.80) | (0.83) | (0.96) |

### Example 4—Measurements of Vital Signs

Participants should rest for at least 5 minutes before vital sign measurement and vital signs should be taken prior to any blood draws. The following vital signs should be measured:
Heart Rate (measured in beats per minute)
Blood Pressure (seated BP will be taken measured in mm Hg)
Temperature [measured in degrees Celsius (° C.) or Fahrenheit (° F.)]

All vital signs above must be measured at all visits. Orthostatic heart rate (HR) and systolic/diastolic BP will also be measured at Visit 1 prior to administration of study drug when the participant is at rest for 5 minutes (preferably supine) and again after standing at 3 minutes for assessment of orthostatic hypotension. Orthostatic hypotension is defined as decrease in SBP of ≥20 mmHg (Stewart et al., Pediatrics, 141, 1-13, 2018). Accompanying tachycardia is defined as change of heart rate increment of ≥40 bpm and absolute orthostatic HR ≥130 bpm (for ages 13 years and younger) (Singer et al., Journal of Pediatrics, 160, 222-226, 2012). Additionally, at Visit 1 and Visit 7 (in the Open-Label Extension Period), all vital signs must be monitored at 1 hour and 2 hours post injection of the study drug. For participants that undergo blood collection for pharmacokinetic analyses at 8, 24, and 48 hours following first dose (at Visit 1 in the Randomized Period and Visit 7 in the Open-Label Extension Period), orthostatic systolic/diastolic BP and HR will be measured prior to each PK blood collection.

### Example 5—Safety

In 57 patients from the trial followed for up to 65 weeks, no withdrawals or discontinuations from the trial for any reason was reported. There were no serious adverse events related to the trial drug. Also, injection site tolerability was generally very good, with a total of 13 AEs related to trial drug or procedures, including 8 injection site reactions occurring among more than 1900 injections. There were no AEs reported on orthostatism. Importantly, no treatment emergent anti-CNP antibodies were detected.

### Example 6—Pharmacokinetics Data

Sparse PK samples are collected in all subjects (if body weight allows) prior to and at 8, 24, 48 h post the initial dose and post-dose at 1 (trough), 2 (any time during the week), 3 (any time during the week), 6 (trough), 9 (any time) and 12 (trough) months+every 3 months for the duration of the study. A population PK model was used to characterize the sparse PK data. A dose related increase in exposure was observed across the four dosing cohorts, 6 μg/kg, 20 μg/kg, 50 μg/kg and 100 μg/kg. Sustained exposure to free CNP was observed, and a half-life for free CNP of ˜110 hours was estimated.

TABLE 4
Predicted median free CNP Cₘₐₓ for the subjects

| | BW 10 kg | BW 35 kg |
|---|---|---|
| | Cₘₐₓ (pg/ml) | Cₘₐₓ (pg/ml) |
| Dose μg/kg/week | Median (9-95^{th} percentile) | Median (9-95^{th} percentile) |
| 6 | 2.9 (1.6-5.0) | 1.8 (1.1-3.3) |
| 20 | 10.4 (5.8-18.9) | 6.6 (3.8-12.6) |
| 50 | 26.5 (15.2-48.1) | 18.3 (10.3-31.5) |
| 100 | 53.1 (30.9-96.4) | 36.6 (20.2-65.9) |

TABLE 5
Estimated half-lives for free CNP in the subjects

| Dose μg/kg/week | Estimated half-life (h) |
|---|---|
| 6 | 101 |
| 20 | 106 |
| 50 | 108 |
| 100 | 110 |

Blood samples for determination of CNP-38 were acidified immediately after collection, using citrate acid buffer, to stabilize the prodrug in the sample thereby avoiding further liberation of CNP-38 from the prodrug. CNP-38 was determined in plasma (heparin) following protein precipitation and solid phase extraction and analyzed using liquid chromatography and tandem mass spectrometry detection. CNP-38 (38 amino acid peptide) was used as reference material and CNP-38 labelled with deuterium (D30—CNP-38) as internal standard (IS). Positive ions were monitored in multiple-reaction monitoring mode. The quantification was performed using the peak area ratios of analyte versus IS. The calibration curve fitting was done by 1/concentration-weighted linear regression. The calibration range in non-acidified plasma was 1.38 to 138 pmol/l.

TABLE 6
Preliminary mean free CNP Levels from NCT04085523

| CO- | | | TIME | MEAN | SD | |
|---|---|---|---|---|---|---|
| HORT | DOSE | VISIT | (days) | (pmol/L) | (pmol/L) | NOBS |
| 1 | 6 μg/kg | WEEK 0 | 0.96 | 1.86 | 1.12 | 17 |
| 1 | 6 μg/kg | WEEK 4 | 27.98 | 1.38 | 0.00* | 7 |
| 1 | 6 μg/kg | WEEK 8 | 55.97 | 1.68 | 0.46 | 7 |
| 1 | 6 μg/kg | WEEK 12 | 79.02 | 1.85 | 0.67 | 8 |
| 1 | 6 μg/kg | WEEK 26 | 181.99 | 1.48 | 0.19 | 7 |
| 1 | 6 μg/kg | WEEK 39 | 271.94 | 2.02 | 0.58 | 8 |
| 1 | 6 μg/kg | WEEK 52 | 363.90 | 1.58 | 0.39 | 7 |
| 2 | 20 μg/kg | WEEK 0 | 1.01 | 3.49 | 1.51 | 22 |
| 2 | 20 μg/kg | WEEK 4 | 27.94 | 3.27 | 2.18 | 10 |
| 2 | 20 μg/kg | WEEK 8 | 56.27 | 3.79 | 1.63 | 9 |
| 2 | 20 μg/kg | WEEK 12 | 84.00 | 3.84 | 3.15 | 9 |
| 2 | 20 μg/kg | WEEK 26 | 182.00 | 2.77 | 1.49 | 11 |
| 2 | 20 μg/kg | WEEK 39 | 273.47 | 5.19 | 3.58 | 8 |
| 3 | 50 μg/kg | WEEK 0 | 1.00 | 10.58 | 3.59 | 15 |
| 3 | 50 μg/kg | WEEK 4 | 27.99 | 7.29 | 2.49 | 10 |
| 3 | 50 μg/kg | WEEK 8 | 56.00 | 9.24 | 5.31 | 9 |
| 3 | 50 μg/kg | WEEK 12 | 83.91 | 7.62 | 3.88 | 10 |
| 4 | 100 μg/kg | WEEK 0 | 0.99 | 15.29 | 7.00 | 18 |

Observations below the lower limit of quantification (LLOQ) have been set to the LLOQ (1.38 pM)
*All observations below LLOQ
NOBS: number of observations at the given week
Time (days): the median time post the 1^{st} dose
Placebo subjects not included

### Example 7—Neutralizing Antibodies

All subjects in the trial are being monitored for binding and, if relevant, neutralizing antibodies to CNP. No anti-CNP binding antibodies have been detected upon 1 to 9 months of repeated weekly exposure to compound (1). Samples are collected in all subjects at screening visit, prior to initial dose, and post-dose at 1, 3, 6, 9 months+every 3 months for the duration of the study. Samples are analyzed using a fully validated anti-CNP antibody assay. All post-dose samples have been confirmed negative for anti-CNP antibodies.

### Assay Description

A sensitive assay was developed to detect, confirm and quasi-quantify binding antibodies against CNP-38 (and CNP-22) and was designed as a bridging ECL (electro-chemiluminescence) immunoassay. Prior to analysis of clinical samples, the assay was fully validated following regulatory requirements from both EMAA and FDA. In this approach, biotinylated and ruthenylated CNP-38 were prepared as a master mix. Samples were incubated at minimum required dilution (MRD) in the master mix to form complex and then plated on an MSD streptavidin plate where the biotinylated CNP-38 could also bind. The samples were then detected by the ruthenium labelled CNP-38 within the bound complexes, using an MSD S600 plate reader. The presence of anti-drug antibody present was determined by comparing the signal to a statistically derived threshold, the assay cut point.

Height of subjects enrolled in the study of Example 3 was measured using a wall-mounted stadiometer. The same equipment was used at all visits for the same child. All instruments were calibrated as required by local requirements or per instrument manufacturer guidance prior to use. These data demonstrate that weekly subcutaneous treatment of achondroplasia patients aged 2 to 10 years with compound (1) at 100 μg CNP-38/kg for a period of 52 weeks or greater has a beneficial effect on bone growth and corresponds to an annualized growth velocity of 5.42 cm/year, which is significantly higher than the annual growth velocity in the placebo treated cohort of 4.35 cm/year (p=0.0218).

TABLE 7
Superiority of compound (1) 100 μg CNP-38/kg/week in AGV
compared to placebo

| Treatment Group | | p-value |
|---|---|---|
| Compound (1) | AGV (cm/year), n | Compound (1) vs. |
| or Placebo | LS Mean [95% CI] | Pooled Placebo) |
| 6 μg/kg/week | 4.09, n = 10 | 0.6004 |
| | [3.34, 4.84] | |
| 20 μg/kg/week | 4.52, n = 11 | 0.7022 |
| | [3.82, 5.22] | |
| 50 μg/kg/week | 5.16, n = 10 | 0.0849 |
| | [4.43, 5.90] | |
| 100 μg/kg/week | 5.42, n = 11 | 0.0218 |
| | [4.74, 6.11] | |
| Pooled Placebo | 4.35, n = 15 | N/A |
| | [3.75, 4.94] | |

Also, for the 100 μg CNP-38/kg/week group, a tendency in the acceleration in AGV was observed in the time period 6 to 12 months, compared to the AGV in 0 to 6 months with a AGV of 5.696 and 5.167 cm/year, respectively.

It was also evident that there was a comparable AGV across age groups that was dose dependent (Table 8).

TABLE 8
Comparable AGV across age groups

| | Age <5 years old | Age ≥5 years old |
|---|---|---|
| Treatment Group | AGV (cm/year), n | AGV (cm/year), n |
| compound (1) | LS Mean, [95% CI] | LS Mean. [95% CI] |
| 6 μg/kg/week | 4.31, n = 3 | 3.79, n = 7 |
| | [2.52, 6.10] | [2.87, 4.71] |
| 20 μg/kg/week | 4.72, n = 5 | 4.29, n = 6 |
| | [3.30, 6.15] | [3.43, 5.15] |
| 50 μg/kg/week | 5.07, n = 5 | 5.33, n = 5 |
| | [3.62, 6.52] | [4.39, 6.26] |
| 100 μg/kg/week | 5.95, n = 3 | 5.12, n = 8 |
| | [4.03, 7.87] | [4.35, 5.90] |
| Pooled Placebo | 4.53 , n = 8 | 4.29, n = 7 |
| | [3.43, 5.63] | [3.44, 5.14] |

Furthermore, compound (1) administered at 100 (g CNP-38/kg/week demonstrated superiority in achondroplasia specific change in height SDS compared to placebo. A description of achondroplasia specific change in height SDS has previously been disclosed (Orphanet J. Rare Dis. 2021; 16(1):522).

TABLE 9

| | | p-value |
|---|---|---|
| Treatment Group | | (compound |
| (compound (1) | Δ Height SDS*, n | (1) vs. |
| Dose Levels or Placebo) | LS Mean [95% CI] | Pooled Placebo) |
| 6 μg/kg/week | −0.04, n = 10 | 0.8207 |
| | [−0.26, 0.17] | |
| 20 μg/kg/week | 0.03, n = 11 | 0.4107 |
| | [−0.17, 0.23] | |
| 50 μg/kg/week | 0.11, n = 10 | 0.1660 |
| | [−0.10, 0.32] | |
| 100 μg/kg/week | 0.22, n = 11 | 0.0283 |
| | [0.02, 0.41] | |
| Pooled Placebo | −0.08, n = 15 | N/A |
| | [−0.25, 0.10] | |

*ACH-specific height SDS

TABLE 10
Overview of treatment-emergent adverse events

| | 6 μg/ | 20 μg/ | 50 μg/ | 100 μg/ | Total |
|---|---|---|---|---|---|
| | kg/week | kg/week | kg/week | kg/week | Placebo |
| | (n = 10) | (n = 11) | (n = 10) | (n = 111) | (n = 15) |
| Subject with | 9 (90.0) | 11 (100.0) | 10 (100.0) | 10 (90.9) | 14 (93.3) |
| Treatment- | | | | | |
| Emergent | | | | | |
| Adverse Events | | | | | |
| (TEAE)* | | | | | |
| Grade 1 | 9 (90.0) | 11 (100.0) | 10 (100.0) | 9 (81.8) | 14 (93.3) |
| Grade 2 | 3 (30.0) | 3 (27.3) | 3 (30.0) | 1 (9.1) | 5 (33.3) |
| Serious TEAE | 1 (10.0) | 0 | 1 (10.0) | 0 | 0 |
| Treatment- | 3 (30.0) | 2 (18.2) | 3 (30.0) | 2 (18.2) | 5 (33.3) |
| Related TEAE | | | | | |
| Achondroplasia- | 3 (30.0) | 4 (36.4) | 5 (50.0) | 1 (9.1) | 9 (60.0) |
| Related TEAE* | | | | | |

*No reported Grade 3 (severe) or Grade 4 (life-threatening) TEAEs.
**Adverse events reported by investigator as related to underlying disease

Surprisingly, treatment with compound (1) was associated with a reduction in the reporting of achondroplasia related treatment emergent adverse events from 600% in the placebo group to 9.1% in the 100 ug/kg/week dose group.

TABLE 11
Treatment-Related Adverse Events

| | 6 μg/ | 20 μg/ | 50 μg/ | 100 μg/ | Total |
|---|---|---|---|---|---|
| | kg/week | kg/week | kg/week | kg/week | Placebo |
| | (n = 10) | (n = 11) | (n = 10) | (n = 111) | (n = 15) |
| Subjects with | 3 (30.0) | 2 (18.2) | 3 (30.0) | 2 (18.2) | 5 (33.3) |
| at Least | | | | | |
| One Treatment- | | | | | |
| Related TEAE | | | | | |
| Injection site | 2 (20.0) | 1 (9.1) | 3 (30.0) | 2(18.2) | 2 (13.3) |
| reaction* | | | | | |
| Abdominal | 0 | 1 (9.1) | 0 | 0 | 0 |
| pain upper | | | | | |
| Overdose | 0 | 0 | 0 | 0 | 1 (6.7) |
| Dizziness | 0 | 0 | 0 | 0 | 1 (6.7) |
| Sleep terror | 0 | 0 | 0 | 0 | 1 (6.7) |
| Urticaria | 1 (10.0) | 0 | 0 | 0 | 0 |

*Injection site reactions includes preferred terms of injection site reaction, injection site pain, injection site erythema, injectio site discoloration, injection site haemorrage and injection site swelling.

Injections with compound (1) were well tolerated with a low frequency of injection site reactions (ISR). Only 11 ISRs in 8 patients occurred with more than 2,000 injections administered. This was comparable to the event rate in the placebo group with 2 subjects reporting each 1 ISR. All of these were reported as mild in severity.

### Example 8—Pharmacokinetics Data and Determination of Minimum Sustained Free CNP Exposure

During the trial, sparse blood samples were collected at different time points in the dosing interval, and the concentration of free CNP-38 was determined using the methodology described in example 6. Sub-setting the blood samples taken at 6.5 to 7.5 days after last dose, the range of free CNP-38 plasma concentration is shown in Table 11 (PK data cut off: 15 Nov. 2022). For weekly administration of compound (1), this therefore represents the sustained minimum plasma free CNP-38 concentration.

TABLE 11

| Dose CNP | Free CNP-38 | Free CNP-38 |
|---|---|---|
| μg/kg/week | Median (range) pmol/L | Mean (SD) pmol/L |
| 6 | 1.7 (1.4-1.9) | 1.7 (0.2) |
| 20 | 2.9 (1.6-4.2) | 2.9 (9.7) |
| 50 | 6.5 (4-16.9) | 7.2 (2.5) |
| 100 | 13.8 (9.4-23) | 14 (4.2) |
| 150 | 22.3 (19.7-25) | — |

The values are provided as pmol/L CNP-38. Free CNP values may be calculated by a multiplication factor of about 30 to about 50%, to take into account of degradation products of free CNP-38 which remain functional, and native CNP. Values for the 150 μg/kg/week dose are simulated with a population PK model for compound (1). The lower value in the range is the lowest free CNP-38 concentration, also referred to as the trough level or trough concentration. The number of subjects contributing with observations at the specified time interval are: 4, 8, 10 and 6 for the 6, 20, 50 and 100 μg/kg dose groups, respectively.

1-63. (canceled)

64. A method of treating a disease which can be treated with CNP in a human patient in need thereof, the method comprising the step of administering a CNP conjugate or pharmaceutically acceptable salt thereof at a unit dose ranging from about 6 μg CNP/kg to at least about 100 μg CNP/kg to the patient.

65. The method of claim 64, wherein the CNP conjugate or pharmaceutically acceptable salt thereof is a compound of formula (Ia) or (Ib):
Z [symbol] L²-L¹-D)ₓ (Ia),
D [symbol] L¹-L²-Z)_{y} (Ib),
wherein
-D is a CNP moiety;
-L¹- is a reversible linker moiety;
-L²- is a single chemical bond or a spacer moiety;
—Z is a polymeric moiety;
x is an integer selected from the group consisting of 1, 2, 3, 4, 5, 6, 7, 8, 9, 10, 11, 12, 13, 14, 15 and 16; and
y is an integer selected from the group consisting of 1, 2, 3, 4 and 5.

66. The method of claim 64, wherein a patient's body weight ranges from about 2 kg to about 80 kg.

67. The method of claim 64, wherein the unit dose is about 100 μg CNP/kg.

68. The method of claim 64, wherein the CNP conjugate or pharmaceutically acceptable salt thereof is administered weekly to the patient.

69. The method of claim 64, wherein the CNP conjugate or pharmaceutically acceptable salt thereof is administered subcutaneously to the patient.

70. The method of claim 64, wherein the patient is a pediatric patient with open bone epiphysis.

71. The method of claim 64, wherein the patient is at least 6 months of age, such as at least 1 year of age or at least 2 years of age.

72. The method of claim 64, wherein the CNP conjugate or pharmaceutically acceptable salt thereof is administered weekly to a human patient with open epiphysis via subcutaneous injection and wherein each administration is associated with a frequency of injection site reaction of less than 3%, such as less than 2%, such as less than 1% or absence of injection site reaction.

73. The method of claim 64, wherein the disease which can be treated with CNP is selected from the group consisting of achondroplasia, hypochondroplasia, short stature, dwarfism, osteochondrodysplasias, thanatophoric dysplasia, osteogenesis imperfecta, achondrogenesis, chondrodysplasia punctata, homozygous achondroplasia, camptomelic dysplasia, congenital lethal hypophosphatasia, perinatal lethal type of osteogenesis imperfecta, short-rib polydactyly syndromes, rhizomelic type of chondrodysplasia punctata, Jansen-type metaphyseal dysplasia, spondyloepiphyseal dysplasia congenita, atelosteogenesis, diastrophic dysplasia, congenital short femur, Langer-type mesomelic dysplasia, Nievergelt-type mesomelic dysplasia, Robinow syndrome, Reinhardt syndrome, acrodysostosis, peripheral dysostosis, Kniest dysplasia, fibrochondrogenesis, Roberts syndrome, acromesomelic dysplasia, micromelia, Morquio syndrome, Kniest syndrome, metatrophic dysplasia, spondyloepimetaphyseal dysplasia, neurofibromatosis, Legius syndrome, LEOPARD syndrome, Noonan syndrome, hereditary gingival fibromatosis, neurofibromatosis type 1, Legius syndrome, cardiofaciocutaneous syndrome, Costello syndrome, SHOX deficiency, idiopathic short stature, growth hormone deficiency, osteoarthritis, cleidocranial dysostosis, craniosynostosis (e.g., Muenke syndrome, Crouzon syndrome, Apert syndrome, Jackson-Weiss syndrome, Pfeiffer syndrome, or Crouzonodermoskeletal syndrome), dactyly, brachydactyly, camptodactyly, polydactyly, syndactyly, dyssegmental dysplasia, enchondromatosis, fibrous dysplasia, hereditary multiple exostoses, hypophosphatemic rickets, Jaffe-Lichtenstein syndrome, Marfan syndrome, McCune-Albright syndrome, osteopetrosis, osteopoikilosis, hemorrhagic shock, hypertension, restenosis, arteriosclerosis, acute decompensated heart failure, congestive heart failure, cardiac edema, nephredema, hepatic edema, acute renal insufficiency, chronic renal insufficiency, glaucoma, elevated intraocular pressure, multiple myeloma, myeloproliferative syndrome, leukemia, plasma cell leukemia, lymphoma, glioblastoma, prostate cancer, bladder cancer, mammary cancer, growth retardation, skull deformities, orthodontic defects, cervical cord compression, spinal stenosis, hydrocephalus, hearing loss due to chronic otitis, cardiovascular disease, neurological disease and obesity.

74. The method of claim 64, wherein the disease which can be treated with CNP is selected from the group consisting of hypophosphatasia, hypochondroplasia, Muenke syndrome, hypertension, osteogenesis imperfecta and achondroplasia.

75. The method of claim 64, wherein the disease which can be treated with CNP is achondroplasia.

76. The method of claim 64, wherein the CNP conjugate or pharmaceutically acceptable salt thereof is administered via a dosing frequency which sustains plasma free CNP concentration at a therapeutic level between successive doses, such as a plasma free CNP concentration of at least about 1 pmol/L.

77. The method of claim 64, wherein the CNP conjugate or pharmaceutically acceptable salt thereof is administered in a regimen to achieve plasma levels of free CNP in which at steady state, troughs range from 1.8 pmol/L to 29 pmol/L and peaks range from 30 pmol/L to 100 pmol/L.

78. The method of claim 64, wherein the ratio of peaks to troughs is no more than 1.5:1, 2:1 or 3:1.

79. The method of claim 64, wherein the treatment reduces the incidence of achondroplasia-related adverse events in a human patient.

80. The method of claim 64, wherein the achondroplasia-related adverse events are selected from the group consisting of sleep apnea syndrome, ear infection, foramen magnum stenosis and kyphosis.

81. The method of claim 64, wherein upon administration of the CNP conjugate or pharmaceutically acceptable salt thereof the incidence of hypotension is less than 10%, such as less than 8%, such as less than 5% or such as less than 3%.

82. A method of increasing growth velocity in a human patient in need thereof, the method comprising the step of administering a CNP conjugate or pharmaceutically acceptable salt thereof of formula (Ia) or (Ib) at a unit dose of 100 μg CNP/kg to the patient.

83. A method of increasing long bone growth in a human patient in need thereof, the method comprising the step of administering a CNP conjugate or pharmaceutically acceptable salt thereof of formula (Ia) or (Ib) at a unit dose of 100 μg CNP/kg, wherein the unit dose is administered weekly to the patient.


SEQUENCE LISTING (105 sequences)

SEQ ID NO: 1 - protein, 22 aa, Homo sapiens
  DISULFID 6..22: note = intrachain disulfide bond
GLSKGCFGLKLDRIGSMSGLGC

SEQ ID NO: 2 - protein, 53 aa, Homo sapiens
  DISULFID 37..53: note = intrachain disulfide bond
DLRVDTKSRAAWARLLQEHPNARKYKGANKKGLSKGCFGLKLDRIGSMSGLGC

SEQ ID NO: 3 - protein, 54 aa, synthetic construct
  REGION 1..54: note = G-CNP-53
  DISULFID 38..54: note = intrachain disulfide bond
GDLRVDTKSRAAWARLLQEHPNARKYKGANKKGLSKGCFGLKLDRIGSMSGLGC

SEQ ID NO: 4 - protein, 54 aa, synthetic construct
  REGION 1..54: note = M-CNP-53
  DISULFID 38..54: note = intrachain disulfide bond
MDLRVDTKSRAAWARLLQEHPNARKYKGANKKGLSKGCFGLKLDRIGSMSGLGC

SEQ ID NO: 5 - protein, 54 aa, synthetic construct
  REGION 1..54: note = P-CNP-53
  DISULFID 38..54: note = intrachain disulfide bond
PDLRVDTKSRAAWARLLQEHPNARKYKGANKKGLSKGCFGLKLDRIGSMSGLGC

SEQ ID NO: 6 - protein, 53 aa, synthetic construct
  REGION 1..53: note = CNP-53 M48N
  DISULFID 37..53: note = intrachain disulfide bond
DLRVDTKSRAAWARLLQEHPNARKYKGANKKGLSKGCFGLKLDRIGSNSGLGC

SEQ ID NO: 7 - protein, 36 aa, synthetic construct
  REGION 1..36: note = CNP-53 with deletion of amino acids 15-31
  DISULFID 20..36: note = intrachain disulfide bond
DLRVDTKSRAAWARGLSKGCFGLKLDRIGSMSGLGC

SEQ ID NO: 8 - protein, 52 aa, synthetic construct
  REGION 1..52: note = CNP-52
  DISULFID 36..52: note = intrachain disulfide bond
LRVDTKSRAAWARLLQEHPNARKYKGANKKGLSKGCFGLKLDRIGSMSGLGC

SEQ ID NO: 9 - protein, 51 aa, synthetic construct
  REGION 1..51: note = CNP-51
  DISULFID 35..51: note = intrachain disulfide bond
RVDTKSRAAWARLLQEHPNARKYKGANKKGLSKGCFGLKLDRIGSMSGLGC

SEQ ID NO: 10 - protein, 50 aa, synthetic construct
  REGION 1..50: note = CNP-50
  DISULFID 34..50: note = intrachain disulfide bond
VDTKSRAAWARLLQEHPNARKYKGANKKGLSKGCFGLKLDRIGSMSGLGC

SEQ ID NO: 11 - protein, 49 aa, synthetic construct
  REGION 1..49: note = CNP-49
  DISULFID 33..49: note = intrachain disulfide bond
DTKSRAAWARLLQEHPNARKYKGANKKGLSKGCFGLKLDRIGSMSGLGC

SEQ ID NO: 12 - protein, 48 aa, synthetic construct
  REGION 1..48: note = CNP-48
  DISULFID 32..48: note = intrachain disulfide bond
TKSRAAWARLLQEHPNARKYKGANKKGLSKGCFGLKLDRIGSMSGLGC

SEQ ID NO: 13 - protein, 47 aa, synthetic construct
  REGION 1..47: note = CNP-47
  DISULFID 31..47: note = intrachain disulfide bond
KSRAAWARLLQEHPNARKYKGANKKGLSKGCFGLKLDRIGSMSGLGC

SEQ ID NO: 14 - protein, 46 aa, synthetic construct
  REGION 1..46: note = CNP-46
  DISULFID 30..46: note = intrachain disulfide bond
SRAAWARLLQEHPNARKYKGANKKGLSKGCFGLKLDRIGSMSGLGC

SEQ ID NO: 15 - protein, 45 aa, synthetic construct
  REGION 1..45: note = CNP-45
  DISULFID 29..45: note = intrachain disulfide bond
RAAWARLLQEHPNARKYKGANKKGLSKGCFGLKLDRIGSMSGLGC

SEQ ID NO: 16 - protein, 44 aa, synthetic construct
  REGION 1..44: note = CNP-44
  DISULFID 28..44: note = intrachain disulfide bond
AAWARLLQEHPNARKYKGANKKGLSKGCFGLKLDRIGSMSGLGC

SEQ ID NO: 17 - protein, 35 aa, synthetic construct
  REGION 1..35: note = CNP-44 with a deletion of amino acids 14-22
  DISULFID 19..35: note = intrachain disulfide bond
AAWARLLQEHPNAGLSKGCFGLKLDRIGSMSGLGC

SEQ ID NO: 18 - protein, 36 aa, synthetic construct
  REGION 1..36: note = CNP-44 with a deletion of amino acids 15-22
  DISULFID 20..36: note = intrachain disulfide bond
AAWARLLQEHPNARGLSKGCFGLKLDRIGSMSGLGC

SEQ ID NO: 19 - protein, 43 aa, synthetic construct
  REGION 1..43: note = CNP-43
  DISULFID 27..43: note = intrachain disulfide bond
AWARLLQEHPNARKYKGANKKGLSKGCFGLKLDRIGSMSGLGC

SEQ ID NO: 20 - protein, 42 aa, synthetic construct
  REGION 1..42: note = CNP-42
  DISULFID 26..42: note = intrachain disulfide bond
WARLLQEHPNARKYKGANKKGLSKGCFGLKLDRIGSMSGLGC

SEQ ID NO: 21 - protein, 41 aa, synthetic construct
  REGION 1..41: note = CNP-41
  DISULFID 25..41: note = intrachain disulfide bond
ARLLQEHPNARKYKGANKKGLSKGCFGLKLDRIGSMSGLGC

SEQ ID NO: 22 - protein, 40 aa, synthetic construct
  REGION 1..40: note = CNP-40
  DISULFID 24..40: note = intrachain disulfide bond
RLLQEHPNARKYKGANKKGLSKGCFGLKLDRIGSMSGLGC

SEQ ID NO: 23 - protein, 39 aa, synthetic construct
  REGION 1..39: note = CNP-39
  DISULFID 23..39: note = intrachain disulfide bond
LLQEHPNARKYKGANKKGLSKGCFGLKLDRIGSMSGLGC

SEQ ID NO: 24 - protein, 38 aa, synthetic construct
  REGION 1..38: note = CNP-38
  DISULFID 22..38: note = intrachain disulfide bond
LQEHPNARKYKGANKKGLSKGCFGLKLDRIGSMSGLGC

SEQ ID NO: 25 - protein, 37 aa, synthetic construct
  REGION 1..37: note = CNP-37
  DISULFID 21..37: note = intrachain disulfide bond
QEHPNARKYKGANKKGLSKGCFGLKLDRIGSMSGLGC

SEQ ID NO: 26 - protein, 37 aa, synthetic construct
  REGION 1..37: note = CNP-37 mit Q1pQ (pQ = pyroglutamate)
  SITE 1: note = MISC_FEATURE - X = pyroglutamate
  DISULFID 21..37: note = intrachain disulfide bond
XEHPNARKYKGANKKGLSKGCFGLKLDRIGSMSGLGC

SEQ ID NO: 27 - protein, 38 aa, synthetic construct
  REGION 1..38: note = G-CNP-37
  DISULFID 22..38: note = intrachain disulfide bond
GQEHPNARKYKGANKKGLSKGCFGLKLDRIGSMSGLGC

SEQ ID NO: 28 - protein, 38 aa, synthetic construct
  REGION 1..38: note = P-CNP-37
  DISULFID 22..38: note = intrachain disulfide bond
PQEHPNARKYKGANKKGLSKGCFGLKLDRIGSMSGLGC

SEQ ID NO: 29 - protein, 38 aa, synthetic construct
  REGION 1..38: note = M-CNP-37
  DISULFID 22..38: note = intrachain disulfide bond
MQEHPNARKYKGANKKGLSKGCFGLKLDRIGSMSGLGC

SEQ ID NO: 30 - protein, 39 aa, synthetic construct
  REGION 1..39: note = PG-CNP-37
  DISULFID 23..39: note = intrachain disulfide bond
PGQEHPNARKYKGANKKGLSKGCFGLKLDRIGSMSGLGC

SEQ ID NO: 31 - protein, 39 aa, synthetic construct
  REGION 1..39: note = MG-CNP-37
  DISULFID 23..39: note = intrachain disulfide bond
MGQEHPNARKYKGANKKGLSKGCFGLKLDRIGSMSGLGC

SEQ ID NO: 32 - protein, 37 aa, synthetic construct
  REGION 1..37: note = CNP-37 M32N
  DISULFID 21..37: note = intrachain disulfide bond
QEHPNARKYKGANKKGLSKGCFGLKLDRIGSNSGLGC

SEQ ID NO: 33 - protein, 38 aa, synthetic construct
  REGION 1..38: note = G-CNP-37 M32N
  DISULFID 22..38: note = intrachain disulfide bond
GQEHPNARKYKGANKKGLSKGCFGLKLDRIGSNSGLGC

SEQ ID NO: 34 - protein, 38 aa, synthetic construct
  REGION 1..38: note = G-CNP-37 K14Q
  DISULFID 22..38: note = intrachain disulfide bond
GQEHPNARKYKGANQKGLSKGCFGLKLDRIGSMSGLGC

SEQ ID NO: 35 - protein, 38 aa, synthetic construct
  REGION 1..38: note = G-CNP-37 K14P
  DISULFID 22..38: note = intrachain disulfide bond
GQEHPNARKYKGANPKGLSKGCFGLKLDRIGSMSGLGC

SEQ ID NO: 36 - protein, 37 aa, synthetic construct
  REGION 1..37: note = G-CNP-37 K14Q, deletion of amino acid 15
  DISULFID 21..37: note = intrachain disulfide bond
GQEHPNARKYKGANQGLSKGCFGLKLDRIGSMSGLGC

SEQ ID NO: 37 - protein, 38 aa, synthetic construct
  REGION 1..38: note = G-CNP-37 K14Q, K15Q
  DISULFID 22..38: note = intrachain disulfide bond
GQEHPNARKYKGANQQGLSKGCFGLKLDRIGSMSGLGC

SEQ ID NO: 38 - protein, 36 aa, synthetic construct
  REGION 1..36: note = CNP-36
  DISULFID 20..36: note = intrachain disulfide bond
EHPNARKYKGANKKGLSKGCFGLKLDRIGSMSGLGC

SEQ ID NO: 39 - protein, 35 aa, synthetic construct
  REGION 1..35: note = CNP-35
  DISULFID 19..35: note = intrachain disulfide bond
HPNARKYKGANKKGLSKGCFGLKLDRIGSMSGLGC

SEQ ID NO: 40 - protein, 34 aa, synthetic construct
  REGION 1..34: note = CNP-34
  DISULFID 18..34: note = intrachain disulfide bond
PNARKYKGANKKGLSKGCFGLKLDRIGSMSGLGC

SEQ ID NO: 41 - protein, 33 aa, synthetic construct
  REGION 1..33: note = CNP-33
  DISULFID 17..33: note = intrachain disulfide bond
NARKYKGANKKGLSKGCFGLKLDRIGSMSGLGC

SEQ ID NO: 42 - protein, 32 aa, synthetic construct
  REGION 1..32: note = CNP-32
  DISULFID 16..32: note = intrachain disulfide bond
ARKYKGANKKGLSKGCFGLKLDRIGSMSGLGC

SEQ ID NO: 43 - protein, 31 aa, synthetic construct
  REGION 1..31: note = CNP-31
  DISULFID 15..31: note = intrachain disulfide bond
RKYKGANKKGLSKGCFGLKLDRIGSMSGLGC

SEQ ID NO: 44 - protein, 30 aa, synthetic construct
  REGION 1..30: note = CNP-30
  DISULFID 14..30: note = intrachain disulfide bond
KYKGANKKGLSKGCFGLKLDRIGSMSGLGC

SEQ ID NO: 45 - protein, 29 aa, synthetic construct
  REGION 1..29: note = CNP-29
  DISULFID 13..29: note = intrachain disulfide bond
YKGANKKGLSKGCFGLKLDRIGSMSGLGC

SEQ ID NO: 46 - protein, 28 aa, synthetic construct
  REGION 1..28: note = CNP-28
  DISULFID 12..28: note = intrachain disulfide bond
KGANKKGLSKGCFGLKLDRIGSMSGLGC

SEQ ID NO: 47 - protein, 38 aa, synthetic construct
  REGION 1..38: note = GHKSEVAHRF-CNP-28
  DISULFID 22..38: note = intrachain disulfide bond
GHKSEVAHRFKGANKKGLSKGCFGLKLDRIGSMSGLGC

SEQ ID NO: 48 - protein, 27 aa, synthetic construct
  REGION 1..27: note = CNP-27
  DISULFID 11..27: note = intrachain disulfide bond
GANKKGLSKGCFGLKLDRIGSMSGLGC

SEQ ID NO: 49 - protein, 27 aa, synthetic construct
  REGION 1..27: note = CNP-27 K4Q, K5Q
  DISULFID 11..27: note = intrachain disulfide bond
GANQQGLSKGCFGLKLDRIGSMSGLGC

SEQ ID NO: 50 - protein, 27 aa, synthetic construct
  REGION 1..27: note = CNP-27 K4R, K5R
  DISULFID 11..27: note = intrachain disulfide bond
GANRRGLSKGCFGLKLDRIGSMSGLGC

SEQ ID NO: 51 - protein, 27 aa, synthetic construct
  REGION 1..27: note = CNP-27 K4P, K5R
  DISULFID 11..27: note = intrachain disulfide bond
GANPRGLSKGCFGLKLDRIGSMSGLGC

SEQ ID NO: 52 - protein, 27 aa, synthetic construct
  REGION 1..27: note = CNP-27 K4S, K5S
  DISULFID 11..27: note = intrachain disulfide bond
GANSSGLSKGCFGLKLDRIGSMSGLGC

SEQ ID NO: 53 - protein, 30 aa, synthetic construct
  REGION 1..30: note = GAN-CNP-27 K4P, K5R
  DISULFID 14..30: note = intrachain disulfide bond
GANGANPRGLSRGCFGLKLDRIGSMSGLGC

SEQ ID NO: 54 - protein, 27 aa, synthetic construct
  REGION 1..27: note = CNP-27 K4R, K5R, K9R
  DISULFID 11..27: note = intrachain disulfide bond
GANRRGLSRGCFGLKLDRIGSMSGLGC

SEQ ID NO: 55 - protein, 27 aa, synthetic construct
  REGION 1..27: note = CNP-27 K4R, K5R, K9R, M22N
  DISULFID 11..27: note = intrachain disulfide bond
GANRRGLSRGCFGLKLDRIGSNSGLGC

SEQ ID NO: 56 - protein, 28 aa, synthetic construct
  REGION 1..28: note = P-CNP-27 K4R, K5R, K9R
  DISULFID 12..28: note = intrachain disulfide bond
PGANRRGLSRGCFGLKLDRIGSMSGLGC

SEQ ID NO: 57 - protein, 28 aa, synthetic construct
  REGION 1..28: note = M-CNP-27 K4R, K5R, K9R
  DISULFID 12..28: note = intrachain disulfide bond
MGANRRGLSRGCFGLKLDRIGSMSGLGC

SEQ ID NO: 58 - protein, 38 aa, synthetic construct; identical to SEQ ID NO: 47
  REGION 1..38: note = Human Serum Albumine Fragment - CNP-27
  DISULFID 22..38: note = intrachain disulfide bond

SEQ ID NO: 59 - protein, 38 aa, synthetic construct
  REGION 1..38: note = Human Serum Albumine Fragment - CNP-27 M22N
  DISULFID 22..38: note = intrachain disulfide bond
GHKSEVAHRFKGANKKGLSKGCFGLKLDRIGSNSGLGC

SEQ ID NO: 60 - protein, 39 aa, synthetic construct
  REGION 1..39: note = methionine - Human Serum Albumine Fragment - CNP-27
  DISULFID 23..39: note = intrachain disulfide bond
MGHKSEVAHRFKGANKKGLSKGCFGLKLDRIGSMSGLGC

SEQ ID NO: 61 - protein, 39 aa, synthetic construct
  REGION 1..39: note = proline - Human Serum Albumine Fragment - CNP-27
  DISULFID 23..39: note = intrachain disulfide bond
PGHKSEVAHRFKGANKKGLSKGCFGLKLDRIGSMSGLGC

SEQ ID NO: 62 - protein, 26 aa, synthetic construct
  REGION 1..26: note = CNP-26
  DISULFID 10..26: note = intrachain disulfide bond
ANKKGLSKGCFGLKLDRIGSMSGLGC

SEQ ID NO: 63 - protein, 25 aa, synthetic construct
  REGION 1..25: note = CNP-25
  DISULFID 9..25: note = intrachain disulfide bond
NKKGLSKGCFGLKLDRIGSMSGLGC

SEQ ID NO: 64 - protein, 24 aa, synthetic construct
  REGION 1..24: note = CNP-24
  DISULFID 8..24: note = intrachain disulfide bond
KKGLSKGCFGLKLDRIGSMSGLGC

SEQ ID NO: 65 - protein, 23 aa, synthetic construct
  REGION 1..23: note = CNP-23
  DISULFID 7..23: note = intrachain disulfide bond
KGLSKGCFGLKLDRIGSMSGLGC

SEQ ID NO: 66 - protein, 23 aa, synthetic construct
  REGION 1..23: note = R-CNP-22
  DISULFID 7..23: note = intrachain disulfide bond
RGLSKGCFGLKLDRIGSMSGLGC

SEQ ID NO: 67 - protein, 24 aa, synthetic construct
  REGION 1..24: note = ER-CNP-22
  DISULFID 8..24: note = intrachain disulfide bond
ERGLSKGCFGLKLDRIGSMSGLGC

SEQ ID NO: 68 - protein, 23 aa, synthetic construct
  REGION 1..23: note = R-CNP-22 K4R
  DISULFID 7..23: note = intrachain disulfide bond
RGLSRGCFGLKLDRIGSMSGLGC

SEQ ID NO: 69 - protein, 24 aa, synthetic construct
  REGION 1..24: note = ER-CNP-22 4KR
  DISULFID 8..24: note = intrachain disulfide bond
ERGLSRGCFGLKLDRIGSMSGLGC

SEQ ID NO: 70 - protein, 24 aa, synthetic construct
  REGION 1..24: note = RR-CNP-22
  DISULFID 8..24: note = intrachain disulfide bond
RRGLSRGCFGLKLDRIGSMSGLGC

SEQ ID NO: 71 - protein, 37 aa, synthetic construct
  REGION 1..37: note = HRGP fragment - CNP-22
  DISULFID 21..37: note = intrachain disulfide bond
GHHSHEQHPHGANQQGLSKGCFGLKLDRIGSMSGLGC

SEQ ID NO: 72 - protein, 38 aa, synthetic construct
  REGION 1..38: note = HRGP fragment - CNP-22
  DISULFID 22..38: note = intrachain disulfide bond
GAHHPHEHDTHGANQQGLSKGCFGLKLDRIGSMSGLGC

SEQ ID NO: 73 - protein, 37 aa, synthetic construct
  REGION 1..37: note = HRGP fragment - CNP-22
  DISULFID 21..37: note = intrachain disulfide bond
GHHSHEQHPHGANPRGLSKGCFGLKLDRIGSMSGLGC

SEQ ID NO: 74 - protein, 36 aa, synthetic construct
  REGION 1..36: note = IgG1(FC) fragment - CNP-22
  DISULFID 20..36: note = intrachain disulfide bond
GQPREPQVYTLPPSGLSKGCFGLKLDRIGSMSGLGC

SEQ ID NO: 75 - protein, 39 aa, synthetic construct
  REGION 1..39: note = Human Serum Albumine - CNP-22
  DISULFID 23..39: note = intrachain disulfide bond
GQHKDDNPNLPRGANPRGLSKGCFGLKLDRIGSMSGLGC

SEQ ID NO: 76 - protein, 37 aa, synthetic construct
  REGION 1..37: note = Human Serum Albumine - CNP-22
  DISULFID 21..37: note = intrachain disulfide bond
GERAFKAWAVARLSQGLSKGCFGLKLDRIGSMSGLGC

SEQ ID NO: 77 - protein, 35 aa, synthetic construct
  REGION 1..35: note = osteocrin NPR C inhibitor fragment - CNP-22
  DISULFID 19..35: note = intrachain disulfide bond
FGIPMDRIGRNPRGLSKGCFGLKLDRIGSMSGLGC

SEQ ID NO: 78 - protein, 40 aa, synthetic construct [sequence omitted]

SEQ ID NO: 79 - protein, 37 aa, synthetic construct
  REGION 1..37: note = IgG1(FC) fragment - CNP-22 K4R
  DISULFID 21..37: note = intrachain disulfide bond
GQPREPQVYTGANQQGLSRGCFGLKLDRIGSMSGLGC

SEQ ID NO: 80 - protein, 36 aa, synthetic construct
  REGION 1..36: note = Human Serum Albumine fragment - CNP-22 K4R
  DISULFID 20..36: note = intrachain disulfide bond
GVPQVSTSTGANQQGLSRGCFGLKLDRIGSMSGLGC

SEQ ID NO: 81 - protein, 37 aa, synthetic construct
  REGION 1..37: note = fibronectin fragment - CNP-22 K4R
  DISULFID 21..37: note = intrachain disulfide bond
GQPSSSSQSTGANQQGLSRGCFGLKLDRIGSMSGLGC

SEQ ID NO: 82 - protein, 37 aa, synthetic construct
  REGION 1..37: note = fibronectin fragment - CNP-22 K4R
  DISULFID 21..37: note = intrachain disulfide bond
GQTHSSGTQSGANQQGLSRGCFGLKLDRIGSMSGLGC

SEQ ID NO: 83 - protein, 37 aa, synthetic construct
  REGION 1..37: note = fibronectin fragment - CNP-22 K4R
  DISULFID 21..37: note = intrachain disulfide bond
GSTGQWHSESGANQQGLSRGCFGLKLDRIGSMSGLGC

SEQ ID NO: 84 - protein, 37 aa, synthetic construct
  REGION 1..37: note = zinc finger fragment - CNP-22 K4R
  DISULFID 21..37: note = intrachain disulfide bond
GSSSSSSSSSGANQQGLSRGCFGLKLDRIGSMSGLGC

SEQ ID NO: 85 - protein, 21 aa, synthetic construct
  REGION 1..21: note = CNP-21
  DISULFID 5..21: note = intrachain disulfide bond
LSKGCFGLKLDRIGSMSGLGC

SEQ ID NO: 86 - protein, 20 aa, synthetic construct
  REGION 1..20: note = CNP-20
  DISULFID 4..20: note = intrachain disulfide bond
SKGCFGLKLDRIGSMSGLGC

SEQ ID NO: 87 - protein, 19 aa, synthetic construct
  REGION 1..19: note = CNP-19
  DISULFID 3..19: note = intrachain disulfide bond
KGCFGLKLDRIGSMSGLGC

SEQ ID NO: 88 - protein, 18 aa, synthetic construct
  REGION 1..18: note = CNP-18
  DISULFID 2..18: note = intrachain disulfide bond
GCFGLKLDRIGSMSGLGC

SEQ ID NO: 89 - protein, 17 aa, synthetic construct
  REGION 1..17: note = CNP-17
  DISULFID 1..17: note = intrachain disulfide bond
CFGLKLDRIGSMSGLGC

SEQ ID NO: 90 - protein, 32 aa, synthetic construct
  REGION 1..32: note = BNP fragment - CNP-17 - BNP- fragment
  DISULFID 10..26: note = intrachain disulfide bond
SPKMVQGSGCFGLKLDRIGSMSGLGCKVLRRH

SEQ ID NO: 91 - protein, 38 aa, synthetic construct; identical to SEQ ID NO: 27
  REGION 1..38: note = CNP-38 L1G
  DISULFID 22..38: note = intrachain disulfide bond

SEQ ID NO: 92 - protein, 37 aa, synthetic construct; identical to SEQ ID NO: 25
  REGION 1..37: note = Ac-CNP-37
  MOD_RES 1: note = ACETYLATION
  DISULFID 21..37: note = intrachain disulfide bond

SEQ ID NO: 93 - protein, 37 aa, synthetic construct
  REGION 1..37: note = CNP-37, X = K or R, with the provision that at least one Xaa is R
  SITE 8: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 10: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 14: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 15: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  SITE 19: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
  DISULFID 21..37: note = intrachain disulfide bond
  SITE 25: note = MISC_FEATURE - X = Lys, Arg, Pro, Ser or Gln, with the provision that at least one of amino acids 8, 10, 14, 15, 19 or 25 is selected from the group consisting of Arg, Pro, Ser and Gln
QEHPNARXYXGANXXGLSXGCFGLXLDRIGSMSGLGC

SEQ ID NO: 94 - protein, 37 aa, synthetic construct
  REGION 1..37: note = mutated CNP-37
  SITE 14: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  SITE 15: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  SITE 19: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
  DISULFID 21..37: note = intrachain disulfide bond
  SITE 25: note = MISC_FEATURE - X is selected from the group consising of Lys, Arg, Pro, Ser and Gln, with the provision that at least one of the amino acids at position 14, 15, 19 and 25 is selected from the group consisting of Arg, Pro, Ser and Gln
QEHPNARKYKGANXXGLSXGCFGLXLDRIGSMSGLGC

SEQ ID NO: 95 - protein, 37 aa, synthetic construct
  REGION 1..37: note = mutated CNP-37
  REGION 14..15: note = MISC_FEATURE - X X is selected from the group consisting of Lys Arg, Arg Lys, Lys Pro, Pro Lys, Ser Ser, Arg Ser, Ser Arg, Gln Lys, Gln Arg, Lys Gln, Arg Gln, Arg Arg and Gln Gln
  DISULFID 21..37: note = intrachain disulfide bond
QEHPNARKYKGANXXGLSKGCFGLKLDRIGSMSGLGC

SEQ ID NO: 96 - protein, 15 aa, Homo sapiens
FGLKLDRIGSMSGLG

SEQ ID NO: 97 - protein, 38 aa, Synthetic construct
  DISULFID 22..38: note = intrachain disulfide bond
  REGION 1..38: note = CNP-38 N6Q, N14Q
LQEHPQARKYKGAQKKGLSKGCFGLKLDRIGSMSGLGC

SEQ ID NO: 98 - protein, 39 aa, Synthetic construct
  DISULFID 23..39: note = intrachain disulfide bond
PGQEHPQARRYRGAQRRGLSRGCFGLKLDRIGSMSGLGC

SEQ ID NO: 99 - protein, 39 aa, synthetic construct
  DISULFID 23..39: note = intrachain disulfide bond
PGQEHPNARRYRGANRRGLSRGCFGLKLDRIGSMSGLGC

SEQ ID NO: 100 - protein, 39 aa, synthetic construct
  DISULFID 23..39: note = intrachain disulfide bond
PGQEHPQARKYKGAQKKGLSKGCFGLKLDRIGSMSGLGC

SEQ ID NO: 101 - protein, 39 aa, synthetic construct; identical to SEQ ID NO: 98
  DISULFID 23..39: note = intrachain disulfide bond
  MOD_RES 1: note = acetylation

SEQ ID NO: 102 - protein, 39 aa, synthetic construct; identical to SEQ ID NO: 30
  DISULFID 23..39: note = intrachain disulfide bond
  MOD_RES 1: note = acetylation
  MOD_RES 39: note = C-terminal NH2

SEQ ID NO: 103 - protein, 39 aa, synthetic construct; identical to SEQ ID NO: 99
  DISULFID 23..39: note = intrachain disulfide bond
  MOD_RES 1: note = acetylation

SEQ ID NO: 104 - protein, 39 aa, synthetic construct; identical to SEQ ID NO: 99
  DISULFID 23..39: note = intrachain disulfide bond
  MOD_RES 1: note = acetylation
  MOD_RES 39: note = C-terminal NH2

SEQ ID NO: 105 - protein, 39 aa, synthetic construct; identical to SEQ ID NO: 98
  DISULFID 23..39: note = intrachain disulfide bond
  MOD_RES 1: note = acetylation
  MOD_RES 39: note = C-terminal NH2